(19) United States
(12) Patent Application Publication
(10) Pub. No.: US 2008/0206810 A1
(43) Pub. Date: Aug. 28, 2008

(54) Truncated St6galnaci Polypeptides and Nucleic Acids

(75) Inventors: Karl F. Johnson, Hatboro, PA (US); David James Hakes, Willow Grove, PA (US); Ge Wei, San Diego, CA (US); Li Liu, Maple Glenn, PA (US); Sami Saribas, Philadelphia, PA (US); Eric R. Sjoberg, San Diego, CA (US); Henrik Clausen, Holte (DK); Eric Paul Bennett, Lyngby (DK); Aliakbar Mobasseri, Voorhees, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Neose Technologies, Inc., Horsham, PA (US)

(21) Appl. No.: 11/597,258

(22) PCT Filed: Jun. 3, 2005
(86) PCT No.: PCT/US2005/019583

§ 371 Date: Mar. 11, 2008

Related U.S. Application Data
(60) Provisional application No. 60/576,433, filed on Jun. 3, 2004.
(60) Provisional application No. 60/650,011, filed on Feb. 4, 2005.

(51) Int. Cl. C12P 21/04; C12N 9/10; C12N 15/11; C12N 15/00; C12N 5/06; C12P 19/26; C12N 5/04; C12N 1/20; C12N 1/00

(52) U.S. Cl. 435/69.1; 435/193; 536/23.2; 435/320.1; 435/325; 435/419; 435/252.3; 435/348; 435/254.11; 435/252.33; 435/252.31; 435/84

(57) ABSTRACT

The present invention features compositions and methods related to truncated mutants of ST6GalNAcI. In particular, the invention features truncated human, mouse, and chicken ST6GalNAcI polypeptides. The invention also features nucleic acids encoding such truncated polypeptides, as well as vectors, host cells, expression systems, and methods of expressing and using such polypeptides.

### CROSS-REFERENCES TO RELATED APPLICATIONS

This application claims the benefit of U.S. Provisional Application No. 60/576,433, filed Jun. 3, 2004 and U.S. Provisional Application No. 60/650,011, filed Feb. 4, 2005; both of which are herein incorporated by reference for all purposes.

### FIELD OF INVENTION

The present invention features compositions and methods related to truncated mutants of ST6GalNAcI. In particular, the invention features truncated human, mouse, and chicken ST6GalNAcI polypeptides. The invention also features nucleic acids encoding such truncated polypeptides, as well as vectors, host cells, expression systems, and methods of expressing and using such polypeptides.

### BACKGROUND OF THE INVENTION

A great diversity of oligosaccharide structures and many types of glycopeptides are found in nature, and these are synthesized, in part, by a large number of glycosyltransferases. Glycosyltransferases catalyze the synthesis of glycolipids, glycopeptides, and polysaccharides, by transferring an activated mono- or oligosaccharide residue to an existing acceptor molecule for the initiation or elongation of the carbohydrate chain. A catalytic reaction is believed to involve the recognition of both the donor and acceptor by suitable domains, as well as the catalytic site of the enzyme.

Many peptide therapeutics, and many potential peptide therapeutics, are glycosylated peptides. The production of a recombinant glycopeptide, as opposed to a recombinant non-glycosylated peptide, requires that a recombinantly-produced peptide is subjected to additional processing steps, either within the cell or after the peptide is produced by the cell, where the processing steps are performed in vitro. The peptide can be treated enzymatically to introduce one or more glycosyl groups onto the peptide, using a glycosyltransferase. Specifically, the glycosyltransferase covalently attaches the glycosyl group or groups to the peptide.

The extra in vitro steps of peptide processing to produce a glycopeptide can be time consuming and costly. This is due, in part, to the burden and cost of producing recombinant glycosyltransferases for the in vitro glycosylation of peptides and glycopeptides to produce glycopeptide therapeutics. As the demand and usefulness of recombinant glycotherapeutics increases, new methods are required in order to more efficiently prepare glycopeptides. Moreover, as more and more glycopeptides are discovered to be useful for the treatment of a variety of diseases, there is a need for methods that lower the cost of their production. Further, there is also a need in the art to develop methods of more efficiently producing recombinant glycopeptides for use in developing and improving glycopeptide therapeutics.

Glycosyltransferases are reviewed in general in International (PCT) Patent Application No. WO03/031464 (PCT/US02/32263), which is incorporated herein by reference in its entirety. One such particular glycosyltransferase that has utility in the development and production of therapeutic glycopeptides is ST6GalNAcI. ST6GalNAcI, or GalNAcα2,6-sialyltransferase, catalyzes the transfer of sialic acid from a sialic acid donor to a sialic acid acceptor. Full length chicken ST6GalNAcI enzyme, for example, is disclosed by Kurosawa et al. (1994, J Biol. Chem. 269:1402-1409). However, the identification of useful mutants of this enzyme, having enhanced biological activity such as enhanced catalytic activity or enhanced stability, has not heretofore been reported.

In the past, there have been efforts to increase the availability of recombinant glycosyltransferases for the in vitro production of glycopeptides. To date, a limited amount of work has been done with respect to recombinant glycosyltransferases that may sometimes be suitable for small-scale production of oligosaccharides or glycopeptides. For example, Kurosawa et al. (1994, J Biol Chem. 269: 1402-1409) describe a truncation mutant of chicken ST6GalNAcI lacking amino acid residues 1-232 of the full-length enzyme. A truncation of mouse ST6GalNAcI was also reported by Kurosawa et al. (2000, J. Biochem., 127:845-854). However, for example, the truncated chicken enzyme described by Kurosawa et al. tacks the substrate specificity of other ST6GalNAcI enzymes and lacks the activity required for “pharmaceutical-scale” processes and reactions, including the production of glycopeptide therapeutics. Therefore, a need still exists for recombinant glycosyltransferases having activity that is suitable for “pharmaceutical-scale” processes and reactions, including the production of glycopeptide therapeutics. In particular, there is a need for recombinant glycosyltransferases having favorable functional and structural characteristics. Further, a need exists for efficient methods of identification and characterization of recombinant glycosyltransferases, as well as for the production of such glycosyltransferases. The present invention addresses and meets these needs.

### BRIEF SUMMARY OF THE INVENTION

The present invention provides an isolated truncated ST6GalNAcI polypeptide that lacks all or a portion of e.g., the ST6GalNAcI signal domain, all or a portion of the ST6GalNAcI transmembrane domain, or all or a portion of the ST6GalNAcI stem domain; with the proviso that said polypeptide is not a chicken ST6GalNAcI polypeptide truncation mutant lacking amino acid residues 1-232. The truncated ST6GalNAcI polypeptides can be e.g., a truncated human ST6GalNAcI, a truncated chicken ST6GalNAcI, or a truncated mouse ST6GalNAcI.

In one embodiment, the truncated ST6GalNAcI polypeptide has at least 90% or 95% identity with a polypeptide selected from SEQ ID NO: 10, SEQ ID NO: 12, SEQ ID NO: 14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, Δ225 of the chicken sequence shown in FIG. 31, SEQ ID NO: 18, Δ30 of the mouse sequence shown in FIG. 31, Δ51 of the mouse sequence shown in FIG. 31, SEQ ID NO:22, SEQ ID NO:24 and Δ200 of the mouse sequence shown in FIG. 31. In another embodiment, the isolated truncated ST6GalNAcI polypeptide comprises an amino acid sequence of SEQ ID NO: 10, SEQ ID NO: 12, SEQ ID NO: 14, Δ35 of the human sequence shown in FIG. 31, Δ72 of the human-sequence shown in FIG. 31 Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, Δ225 of the chicken sequence shown in FIG. 31, SEQ ID NO: 18, Δ30 of the mouse sequence shown in FIG. 31, Δ51 of the mouse sequence shown in FIG. 31, SEQ ID NO:22, SEQ ID NO:24 and Δ200 of the mouse sequence shown in FIG. 31.

The truncated ST6GalNAcI polypeptide can be a fusion protein and comprise a tag polypeptide, such as, e.g., a maltose binding protein, a histidine tag, a Factor IX tag, a glutathione-S-transferase tag, a FLAG-tag, and a starch binding domain tag.

In another aspect, the invention include isolated nucleic acid molecules that encode the truncated ST6GalNAcI polypeptides described above. The nucleic acids can be operably linked to a promoter/regulatory sequence or can be part of an expression vector. The invention also include host cells that comprise expression vectors that encode the truncated ST6GalNAcI polypeptides described above. Such host cells can be eukaryotic or prokaryotic host cells. Eukaryotic cells include e.g., mammalian cells, insect cells, and a fungal cells. Insect cells can include e.g., SF9 cells, SF9+ cells, Sf21 cells, HIGH FIVE cells, or Drosophila Schneider S2 cells. Preferred prokaryotic cells include e.g., E. coli cells and B. subtilis cells. The invention also include methods of using the host cells to produce truncated ST6GalNAcI polypeptides, by growing the recombinant host cells under conditions suitable for expression of the truncated ST6GalNAcI polypeptide.

In another aspect, the present invention includes a method of catalyzing the transfer of a sialic acid moiety to an acceptor moiety using the truncated ST6GalNAcI polypeptides described above to mediate the covalent linkage of said sialic acid moiety to said acceptor moiety, thereby catalyzing the transfer of a sialic acid moiety to an acceptor moiety.

In a further aspect, the invention provides a method of catalyzing the transfer of a sialic acid moiety to an acceptor moiety by incubating the truncated ST6GalNAcI polypeptides described above with a cytidinemonophosphate-sialic acid (CMP-NAN) sialic acid donor and an asialo bovine submaxillary mucin acceptor moiety, wherein said polypeptide mediates the transfer of said sialic acid moiety from said CMP-NAN sialic acid donor to said asialo bovine submaxillary mucin acceptor, thereby catalyzing the transfer of a sialic acid moiety to an acceptor moiety. In preferred embodiment, the acceptor is a polypeptide acceptor, such as e.g., erythropoietin, human growth hormone, granulocyte colony stimulating factor, interferons alpha, -beta, and -gamma, Factor IX, follicle stimulating hormone, interleukin-2, erythropoietin, anti-TNF-alpha, and a lysosomal hydrolase. In other embodiments, the polypeptide acceptor is a glycopeptide. In a further preferred embodiment, the sialic acid moiety comprises a polyethylene glycol moiety. In a still further embodiment the method is carried out on a commercial scale to make commercial scale amounts of a sialylated product, e.g., a sialylated glycoprotein or glycopeptide.

In another aspect, the invention provides an isolated truncated human or chicken ST6GalNAcI polypeptide that lacks all or a portion of the ST6GalNAcI signal domain, with the proviso that said polypeptide is not a chicken ST6GalNAcI polypeptide truncation mutant lacking amino acid residues 1-232. In other embodiments, the truncated human or chicken ST6GalNAcI polypeptide can additionally lack all or a portion of the ST6GalNAcI transmembrane domain or can lack all or a portion of the ST6GalNAcI stem domain.

In some embodiments, the truncated human or chicken ST6GalNAcI polypeptide includes an amino acid sequence with at least 90% or 95% identity to the following: SEQ ID NO: 10, SEQ ID NO: 12, SEQ ID NO: 14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, and Δ225 of the chicken sequence shown in FIG. 31. In a further embodiment, the truncated human or chicken ST6GalNAcI polypeptide includes an amino acid sequence selected from the group consisting of SEQ ID NO:10, SEQ ID NO:12, SEQ ID NO:14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, and Δ225 of the chicken sequence shown in FIG. 31.

The truncated human or chicken ST6GalNAcI polypeptide can be a fusion protein and comprise a tag polypeptide, such as, e.g., a maltose binding protein, a histidine tag, a Factor IX tag, a glutathione-S-transferase tag, a FLAG-tag, and a starch binding domain tag.

In another aspect, the invention include isolated nucleic acid molecules that encode the truncated human or chicken ST6GalNAcI polypeptides described above. The nucleic acids can be operably linked to a promoter/regulatory sequence or can be part of an expression vector. The invention also includes host cells that comprise expression vectors that encode the truncated human or chicken ST6GalNAcI polypeptides described above. Such host cells can be eukaryotic or prokaryotic host cells. Eukaryotic cells include, e.g., mammalian cells, insect cells, and a fungal cells. Insect cells can include e.g., SF9 cells, SF9+ cells, Sf2 cells, HIGH FIVE cells, or Drosophila Schneider S2 cells. Preferred prokaryotic cells include e.g., E. coli cells and B. subtilis cells. The invention also include methods of using the host cells to produce truncated human or chicken ST6GalNAcI polypeptides, by growing the recombinant host cells under conditions suitable for expression of the truncated human or chicken ST6GalNAcI polypeptide.

In another aspect, the present invention includes a method of catalyzing the transfer of a sialic acid moiety to an acceptor moiety using the truncated human or chicken ST6GalNAcI polypeptides described above to mediate the covalent linkage of said sialic acid moiety to said acceptor moiety, thereby catalyzing the transfer of a sialic acid moiety to an acceptor moiety.

In a further aspect, the invention provides a method of catalyzing the transfer of a sialic acid moiety to an acceptor moiety by incubating the truncated human or chicken ST6GalNAcI polypeptides described above with a cytidinemonophosphate-sialic acid (CMP-NAN) sialic acid donor and an asialo bovine submaxillary mucin acceptor moiety, wherein said polypeptide mediates the transfer of said sialic acid moiety from said CMP-NAN sialic acid donor to said asialo bovine submaxillary mucin acceptor, thereby catalyzing the transfer of a sialic acid moiety to an acceptor moiety. In preferred embodiment, the acceptor is a polypeptide acceptor, such as e.g., erythropoietin, human growth hormone, granulocyte colony stimulating factor, interferons alpha, -beta, and -gamma, Factor IX, follicle stimulating hormone, interleukin-2, erythropoietin, anti-TNF-alpha, and a lysosomal hydrolase. In other embodiments, the polypeptide acceptor is a glycopeptide. In a further preferred embodiment, the sialic acid moiety comprises a polyethylene glycol moiety. In a still further embodiment the method is carried out on a commercial scale to make commercial scale amounts of a sialylated product, e.g., a sialylated glycoprotein or glycopeptide.

### BRIEF DESCRIPTION OF THE DRAWINGS

For purpose of illustrating the invention, there are depicted in the drawings certain embodiments of the invention. However, the invention is not limited to the precise arrangements and instrumentalities of the embodiments depicted in the drawings.

FIG. 1 is a diagram illustrating the location of restriction enzyme cleavage sites within the mouse ST6GalNAcI truncation mutants Δ31, Δ51, Δ126, Δ185, and Δ200 (referenced as D32, E52, S127, S186 and S201 in the illustration, respectively). The figure also illustrates the respective size, in bp, of each construct.

FIG. 2 is an image of an electrophoretic gel DNA fragments of 1488 bp, 1428 bp, 1203 bp, 1026 bp, and 981 bp, corresponding respectively to D32, E52, S127, S186, and S201 of N-terminal amino acid truncated ST6GalNAcI nucleic acids. Lane 1, bp ladder; lane 2, D32; lane 3, E52; lane 4, S127; lane 5, S186; lane 6, S201.

FIG. 3 is an image of an electrophoretic gel containing DNA from restriction enzyme digestions using endonucleases BamHI/XhoI for E52, S127, S186 and S201 mouse ST6GalNAcI constructs and HindIII/XhoI for the D32 mouse ST6GalNAcI construct. DNA fragments of approximately 1.5 to 1.0 Kb correspond to different truncation mutants of ST6GalNAcI. The larger fragment visible near 6.0 Kb is pCWin2-MBP. Lane 1, bp ladder; upper lanes 2-4, E52; upper lanes 5-7, S127; upper lanes 8-10, S186; upper lanes 11-12, S201; lower lanes 2-5, D32, lower lanes 7-9, MBP-pCWin2.

FIG. 4 is an image of an electrophoretic gel illustrating the results of the screening of recombinant colonies DH5α/pCWin2-MBP-ST6GalNAcI, using HindIII/XhoI restriction enzymes to digest the D32 construct and BamHI/XhoI to digest the constructs E52, S127, S186 and S201. All 4 colonies from each truncation (numbered 1 through 4) released a fragment of approximately 1.5 to 1.0 Kb corresponding respectively to D32, E52, S127, S186 and S201 of ST6GalNAcI and a larger fragment around 6.0 Kb representing the MBP-pCWin2 vector. Lane 1, bp ladder. Upper lanes 1, 3, 5, 7 and 9, uncut D32/vector; upper lanes 11, 13, 15, uncut E52/vector; upper lanes 17 and 19, uncut S127/vector; upper lanes 2, 4, 6, and 8, cut D32; upper lanes 10, 12 and 14, cut E52; upper lanes 16 and 18, cut S127; lower lanes 1, 3 and 5, uncut S127; lower lanes 7, 9 and 11, uncut S186; lower lanes 13, 15, 17 and 19, uncut S201; lower lanes 2 and 4, cut S127; lower lanes 6, 8, 10 and 12, cut S186; lower lanes 14, 16 and 18, cut S201.

FIG. 5 is an image of an electrophoretic gel illustrating restriction digestion analysis on plasmid DNA isolated from colonies #1 thru #2 of each construct DH5α/pCWin2-MBP-ST6GalNAcI. Plasmid DNA was double digested with NdeI/HindIII enzymes. All colonies except for the 1)₃₂-containing colonies released a single band around 2.5 Kb (D32 released two fragments) which is indicative of the MBP-ST6GalNAcI insert, while the larger expected band around 5.0 Kb corresponds to the pCWin2 vector. M=bp ladder. Lanes 1, 3=D32; 5, 7=E52; 11, 22=S127; 12, 14=S186 and 16, 18 S201, and all contain uncut DNA. Lanes 2, 4=D32; 6, 8=E52; 10, 21=S127; 13, 15 S186 and 17, 19=S201, and all contain digested DNA.

FIG. 6 is an image of an electrophoretic gel illustrating diagnostic restriction enzyme digestion of construct JM109/pCWin2-MBP-ST6GalNAcI, using NdeI/XhoI restriction enzymes. All colonies, with the exception of D32, released a fragment around 2.5 Kb corresponding to truncated MBP-ST6GalNAcI fusion protein (D32 released two fragments). Fragments at 5.0 Kb correspond to the pCWin2 vector. MW=bp ladder. Lanes 1, 3=D32; lanes 5, 7=E52; lanes 9, 11=S127; lanes 13, 15=S186; lanes 17 and 19=S201, and all contain uncut DNA. Lanes 2, 4=D32; lanes 6, 8=E52; lanes 10, 12=S127; lanes 14, 16=S186; lanes 18 and 20=S201 and all contain digested DNA.

FIG. 7 is an image of an electrophoretic protein gel illustrating the presence of polypeptides corresponding to the expected size of the respective mouse ST6GalNAcI truncation mutants present in cell lysate and inclusion bodies for the cells harboring the respective DNA constructs. Lane MW contains a MW marker. Each “lane 1” contains D32, each “lane 2” contains E52, each “lane 3,” contains S127, each “lane 4” contains S186, and each “lane 5” contains S201.

FIG. 8 is an image of an electrophoretic protein gel illustrating the expression of truncated forms of mouse ST6GalNAcI as an MBP fusion protein in lysates and inclusion bodies obtained from JM109 cells. Lane MW contains a MW marker. Each “lane I” contains D32, each “lane 2” contains E52, each “lane 3” contains S127, each “lane 4” contains S186, and each “lane 5” contains S201.

FIG. 9 is an image of an electrophoretic protein gel illustrating the expression of MBP-ST6GalNAcI in JM109 and W3110/pCWin2 MBP-ST6GalNAcI constructs S186 and S201. Lane MW contains a MW marker Lane 1 contains S186 from w3110 #11, 1.0 mg.ml; lane 2 contains S8186 from w3110 #11, 0.1 mg/ml; lane 3 contains S186 from JM109 #11, 1.0 mg/ml; lane 4 contains S186 from JM109 #11, 0.1 mg/ml; lane 5 contains S201 from w3110 #8, 1.0 mg.ml; lane 6 contains S201 from w3110 #8, 0.1 mg/ml; lane 7 contains S201 from JM109 #8, 1.0 mg/ml; lane 8 contains S201 from JM109 #8, 0.1 mg/ml.

FIG. 10 is an image of a mass spectrometric depiction of the transfer of sialic acid to a GalNAc−O-G-CSF acceptor by bacterially-isolated, refolded ST6GalNAcI construct S201. Panel A illustrates a sample taken at 24 hours, Panel B illustrates a sample taken at 48 hours, Panel C illustrates a sample taken at 2 days, and Panel D illustrates a sample taken at 5 days.

FIG. 11 is an image of an electrophoretic gel confirming the human ST6GalNAcI inserts of EST clones by restriction enzymatic digestion. Lanes 1-3, clone#1-3 of EST clone#4816713 digested by EcoR I; Lane 4, 1-Kb ladder; lanes 5-6, clone#1-3 of EST clone#6300955 digested by EcoR I and Xho I.

FIG. 11 is an image of an electrophoretic gel confirming the human ST6GalNAcI inserts of EST clones by restriction enzymatic digestion. Lanes 1-3, clone#1-3 of EST clone#4816713 digested by EcoR I Lane 4, 1-Kb ladder, lanes 5-6, clone#1-3 of EST clone#6300955 digested by EcoR I and Xho I.

FIG. 12 is a diagram illustrating an alignment of cDNA sequences of the #4816713 and clone#6300955 human ST6GalNAcI EST clones clones.

FIG. 13 is an image of an electrophoretic gel illustrating the EcoRI restriction digestion of pCR-hST6-N and pCR-hST6-C of all six human ST6GalNAcI clones containing the correct sizes cDNA insert. Lanes 1-6 contain a restriction digest of six pCR-hST6-N clones; lanes 7-12 contain a restriction digest of six pCR-hST6-C clones.

FIG. 14 is an image of an electrophoretic gel illustrating restriction enzyme digestions of pcDNA3.1-hST6GalNAcI. Panel A: Lane 1, 1-Kb ladder; lanes 2-7, pcDNA3.1-hST6GalNAcI clone #1-6. Panel B: illustration of restriction enzyme map of pcDNA3.1-hST6GalNAcI.

FIG. 15 illustrates the nucleotide and amino acid sequences of pcDNA3.1(+)-hST6GalNAcI-NIC#1.

FIG. 16 is a cartoon depicting the domain structures and the various truncation mutants of human ST6GalNAcI.

FIG. 17A is a plasmid map of the pAcGP67-B baculovirus transfer vector.

FIG. 17B is a map illustrating the cloning site of the pAcGP67-B baculovirus transfer vector.

FIG. 18 is a graph depicting ST6GalNAcI activities in Sf9 cell culture medium for K36, K125 and S258 human ST6GalNAcI constructs, and for pTS103.

FIG. 19A illustrates the nucleotide and amino acid sequences of mouse ST6GalNAcI from pTS103.

FIG. 19B is a cartoon depicting the domain structures and the various truncation mutants of mouse ST6GalNAcI.

FIG. 20A is a plasmid map of the pFastBac1 vector.

FIG. 20B is a map of the polycloning sites of the pFastBac-1-gp vector.

FIG. 21 is an image of an electrophoretic gel illustrating plasmid DNA subjected to EcoRI and XhoI restriction digestions to release mouse ST6GalNAcI DNA inserts from pFastBac-1-gp-mST6GalNAcI. Lanes 1-4, clones# 1-4 of S127 truncation mutant; lanes 5-8, clones #1-4 of S186 truncation mutant; lane 9, 1 kb ladder.

FIG. 22A is a diagram of the primer pairs on the pFastBac-1 bacmid.

FIG. 22B is an image of an electrophoretic gel illustrating PCR screening of mouse ST6GalNAcI cDNA in the bacmid DNA. Electrophoresis of the PCR products was conducted on a 1% agarose gel. Lane 1, 1-kb ladder; lanes 2-9, clones 1-8 of the recombinant bacmid DNA.

FIG. 23 is an image of an electrophoretic gel illustrating analysis of mouse ST6GalNAcI bacmid DNA on a 1% agarose gel. Lane 1, 1-kb ladder; lane 2, S186#3; lane 3, S186#4; lane 4, S127#5; lane 5, S12746.

FIG. 24 is a graph depicting ST6GalNAcI activities in Sf9 cell culture medium for mouse ST6GalNAcI constructs S127#5, S127#6, S186#3, S186#4, and for the pTS103 plasmid.

FIG. 25 is a table depicting the titer calculations of viral stocks for use in the screening of chicken ST6GalNAcI truncated mutant constructs.

FIG. 26 illustrates the full-length nucleic acid sequence of chicken ST6GalNAcI.

FIG. 27 illustrates the amino acid sequence as translated based on the DNA sequence of FIG. 26.

FIG. 28 illustrates the nucleic acid sequence of full length chicken ST6GalNAcI as set forth in GenBank Accession Number X74946.

FIG. 29 illustrates the nucleic acid sequence of K232 chicken ST6GalNAcI.

FIG. 30 illustrates the amino acid sequence of K232 truncated chicken ST6GalNAcI.

FIG. 31 is a sequence comparison of human, mouse and chicken ST6GalNAcI amino acid sequences. The starting residues for Δ48, Δ152, Δ225 and Δ232 mutants—amino acids Q49, V153, L226 and T233, respectively—are surrounded by boxes.

FIG. 32 is an image of an electrophoretic protein gel illustrating the sialylPEGylation of G-CSF by Δ48, Δ152, Δ225 mutant ST6GalNAcI enzymes. Lane 1, MW marker; lane 2, G-CSF sialylPEGylated with Δ48(MOI=0.8, 35.6 U/L); lane 3, G-CSF sialylPEGylated with Δ152 (MOI=1.43, 39.5 U/L); lane 4, G-CSF sialylPEGylated with Δ232 (MOI=0.531, 0 U/L); lane 5, G-CSF sialylPEGylated with K232 VS4-001 ST6GalNAcI (supernatant); lane 6, G-CSF sialylPEGylated with K232 VS4-001 ST6GalNAcI (purified); lane 7, G-CSF sialylPEGylated with Δ48(MOI=0.2, 35.4 U/L); lane 8, G-CSF sialylPEGylated with Δ152 (MOI=0.356, 39.9 U/L); lane 9, G-CSF sialylPEGylated with Δ232 (MOI=0.133, 0 U/L); lane 10, G-CSF sialylPEGylated with Δ232 (MOI=0.133, 0 U/L); lane 11, G-CSF sialylPEGylated with K232 VS4-001 ST6GalNAcI (purified); lane 12, MW marker.

FIG. 33 is an image of an electrophoretic protein gel illustrating the sialylPEGylation of G-CSF by Δ48, Δ152, Δ225 mutant ST6GalNAcI enzymes. Lane 1, MW marker; lane 2, G-CSF sialylPEGylated with Δ48(MOI=0.8, 35.6 U/L); lane 3, G-CSF sialylPEGylated with Δ48(MOI=0.2, 35.4 U/L); lane 4, G-CSF sialylPEGylated with Δ152 (MOI=1.43, 39.5 U/L); lane 5, G-CSF sialylPEGylated with Δ152 (MOI=0.356, 39.9 U/L); lane 6, G-CSF sialylPEGylated with Δ225 (27.9 U/L); lane 7, G-CSF sialylPEGylated with K232 VS4-001 ST6GalNAcI (purified); lane 8, MW marker.

FIG. 34 provides full length amino acid sequences for A) human ST6GalNAcI and for B) chicken ST6GalNAcI, and C) a sequence of the mouse ST6GalNAcI protein beginning at residue 32 of the native mouse protein.

FIG. 35 provides a schematic of a number of preferred human ST6GalNAcI truncation mutants.

FIG. 36 shows a schematic of MBP fusion proteins including the human ST6GalNAcI truncation mutants.

FIG. 37 shows the position of paired and unpaired cysteine residues in the human ST6GalNAcI protein. Single and double cysteine substitution are also shown, e.g., C280S, C362S, C362T, (C280S+C362S), and (C280S+C362T).

FIG. 38 shows ST6GalNAcI activities of human turncated proteins. Activities were determined in samples obtained from a bacculoviral system.

FIG. 39 shows amino acid sequence alignments of three ST6GalNAcI enzymes: Human, chicken and mouse. The original human enzyme truncation was at Δ35 (K36) position right after membrane spanning region. In addition to earlier human ST6GalNAcI truncations, here 6 more human enzyme truncations were designed and generated. The first one Δ72 (T73) was based on protease cleavage and the rest were designed based on homologous regions among the three or two enzymes. The last truncation Δ272 (G273) was analogous to early chicken ST6GalNAcI truncation. The arrows indicate the truncations in the human protein. The figure also shows an alignment of the human sequence with the mouse and chicken proteins and identifies identical and conserved amino acid residues between the proteins.

FIG. 40 shows schematic of a three way fusion between a gp67 secretion peptide, an ST6GalNAcI coding sequence, and an SBD coding sequence. The fusion proteins were expressed in baculovirus, purified on a cyclodextrin column, and assayed for enzymatic activity.

### DETAILED DESCRIPTION OF THE INVENTION

The compositions and methods of the present invention encompass truncation mutants of human ST6GalNAcI, mouse ST6GalNAcI and chicken ST6GalNAcI, isolated nucleic acids encoding these proteins, and methods of their use. ST6GalNAcI polypeptides catalyze the transfer of sialic acid from a sialic acid donor to a sialic acid acceptor.

The glycosyltransferase ST6GalNAcI is an essential reagent for glycosylation of therapeutic glycopeptides. Additionally, ST6GalNAcI is an important reagent for research and development of therapeutically important glycopeptides and oligosaccharide therapeutics. ST6GalNAcI is typically isolated and purified from natural sources, or from tedious and costly in vitro and recombinant sources. The present invention provides compositions and methods relating to simplified and more cost-effective methods of production of ST6GalNAcI enzymes. In particular, the present invention provides compositions and methods relating to truncated ST6GalNAcI enzymes that have improved and useful properties in comparison to their full-length enzyme counterparts.

Truncated glycosyltransferase enzymes of the present invention are useful for in vivo and in vitro preparation of glycosylated peptides, as well as for the production of oligosaccharides containing the specific glycosyl residues that can be transferred by the truncated glycosyltransferase enzymes of the present invention. This is because it is shown for the first time herein that truncated forms of ST6GalNAcI polypeptides possess biological activities comparable to, and in some instances, in excess of their full-length polypeptide counterparts. The present application also discloses that such truncation mutants not only possess biological activity, but also that the truncation mutants may have enhanced properties of solubility, stability and resistance to proteolytic degradation.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

Certain abbreviations are used herein as are common in the art, such as: “Ac” for acetyl, “Glc” for glucose; “Glc” for glucosamine; “GlcA for glucuronic acid; “IdoA” for iduronic acid; “GlcNAc” for N-acetylglucosamine; “NAN” or “sialic acid” or “SA” for N-acetyl neuraminic acid; “UDP” for uridine diphosphate; “CMP” for cytidine monophosphate.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Encoding” refers to the inherent property of specific sequences of nucleotides in a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

An “affinity tag” is a peptide or polypeptide that may be genetically or chemically fused to a second polypeptide for the purposes of purification, isolation, targeting, trafficking, or identification of the second polypeptide. The “genetic” attachment of an affinity tag to a second protein may be effected by cloning a nucleic acid encoding the affinity tag adjacent to a nucleic acid encoding a second protein in a nucleic acid vector.

As used herein, the term “glycosyltransferase,” refers to any enzyme/protein that has the ability to transfer a donor sugar to an acceptor moiety.

A “sugar nucleotide-generating enzyme” is an enzyme that has the ability to produce a sugar nucleotide. Sugar nucleotides are known in the art, and include, but are not limited to, such moieties as UDP-Gal, UDP-GalNAc, and CMP-NAN.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCP or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides. However, the terms “nucleic acid” and “polynucleotide” are used interchangeably herein.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleic acid sequences: the left-hand end of a single-stranded nucleic acid sequence is the 5′ end; the left-hand direction of a double-stranded nucleic acid sequence is referred to as the 5′-direction.

A first defined nucleic acid sequence is said to be “immediately adjacent to” a second defined nucleic acid sequence when, for example, the last nucleotide of the first nucleic acid sequence is chemically bonded to the first nucleotide of the second nucleic acid sequence through a phosphodiester bond. Conversely, a first defined nucleic acid sequence is also said to be “immediately adjacent to” a second defined nucleic acid sequence when, for example, the first nucleotide of the first nucleic acid sequence is chemically bonded to the last nucleotide of the second nucleic acid sequence through a phosphodiester bond.

A first defined polypeptide sequence is said to be “immediately adjacent to” a second defined polypeptide sequence when, for example, the last amino acid of the first polypeptide sequence is chemically bonded to the first amino acid of the second polypeptide sequence through a peptide bond. Conversely, a first defined polypeptide sequence is said to be “immediately adjacent to” a second defined polypeptide sequence when, for example, the first amino acid of the first polypeptide sequence is chemically bonded to the last amino acid of the second polypeptide sequence through a peptide bond.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “percent identity” is used synonymously with “homology.” The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul et al. (1990, J. Mol. Biol. 215:403-410), and can be accessed, for example, at the BLAST site of the National Center for Biotechnology Information (NCBI) world wide web site at the National Library of Medicine (NLM) at the National Institutes of Health (NIH). BLAST nucleotide searches can be performed with the NBLAST program (designated “blastn” at the NCBI web site), using the following parameters: gap penalty 5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used as available on the website of the National Center for Biotechnology Information of the National Library of Medicine at the National Institutes of Health.

The percent identity between two sequences can be determined using techniques similar to those described above, with or without allowing gaps. In calculating percent identity, typically exact matches are counted.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. A “polypeptide,” as the term is used herein, therefore refers to any size polymer of amino acid residues, provided that the polymer contains at least two amino acid residues.

The term “protein” typically refers to large peptides, also referred to herein as “polypeptides.” The term “peptide” typically refers to short polypeptides. However, the terms “peptide,” “protein” and “polypeptide” are used interchangeably herein. For example, the term “peptide” may refer to an amino acid polymer of three amino acids, as well as an amino acid polymer of several hundred amino acids.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “therapeutic peptide” as the term is used herein refers to any peptide that is useful to treat a disease state or to improve the overall health of a living organism. A therapeutic peptide may effect such changes in a living organism when administered alone, or when used to improve the therapeutic capacity of another substance. The term “therapeutic peptide” is used interchangeably herein with the terms “therapeutic polypeptide” and “therapeutic protein.”

A “reagent peptide” as the term is used herein refers to any peptide that is useful in food biochemistry, bioremediation, production of small molecule therapeutics, and even in the production of therapeutic peptides. Typically, reagent peptides are enzymes capable of catalyzing a reaction to produce a product useful in any of the aforementioned areas. The term “reagent peptide” is used interchangeably herein with the terms “reagent polypeptide” and “reagent protein.”

A “glycopeptide” as the term is used herein refers to a peptide having at least one carbohydrate moiety covalently linked thereto. It will be understood that a glycopeptide may be a “therapeutic glycopeptide,” as described above. The term “glycopeptide” is used interchangeably herein with the terms “glycopolypeptide” and “glycoprotein.”

A “vector” is a composition of matter which comprises an isolated nucleic acid and which can be used to deliver the isolated nucleic acid to the interior of a cell. Numerous vectors are known in the art including, but not limited to, linear nucleic acids, nucleic acids associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant nucleic acid comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant nucleic acid.

A “multiple cloning site” as the term is used herein is a region of a nucleic acid vector that contains more than one sequence of nucleotides that is recognized by at least one restriction enzyme.

An “antibiotic resistance marker” as the term is used herein refers to a sequence of nucleotides that encodes a protein which, when expressed in a living cell, confers to that cell the ability to live and grow in the presence of an antibiotic.

As used herein, the term “ST6GalNAcI” refers to N-acetylgalactosamine-α2,6-sialyltransferase I.

As the term is used herein, a “truncated” form of a peptide refers to a peptide that is lacking one or more amino acid residues as compared to the full-length amino acid sequence of the peptide. For example, the peptide “NH2-Ala-Glu-Lys-Leu-COOH” is an N-terminally truncated form of the frill-length peptide “NH2-Gly-Ala-Glu-Lys-Leu-COOH.” The terms “truncated form” and “truncation mutant” are used interchangeably herein. By way of a non-limiting example, a truncated peptide is a ST6GalNAcI polypeptide comprising an active domain, a stem domain, a transmembrane domain, and a signal domain, wherein the signal domain is lacking a single N-terminal amino acid residue as compared to the full length ST6GalNAcI.

The term “saccharide” refers in general to any carbohydrate, a chemical entity with the most basic structure of (CH₂O)ₙ. Saccharides vary in complexity, and may also includes nucleic acid, amino acid, or virtually any other chemical moiety existing in biological systems

“Monosaccharide” refers to a single unit of carbohydrate of a defined identity.

“Oligosaccharide” refers to a molecule consisting of several units of carbohydrates of defined identity. Typically, saccharide sequences between 2-20 units may be referred to as oligosaccharides.

“Polysaccharide” refers to a molecule consisting of many units of carbohydrates of defined identity. However, any saccharide of two or more units may correctly be considered a polysaccharide.

As used herein, a saccharide “donor” is a moiety that can provide a saccharide to a glycosyltransferase so that the glycosyltransferase may transfer the saccharide to a saccharide acceptor. By way of a non-limiting example, a GalNAc donor may be UDP-GalNAc.

As used herein, a saccharide “acceptor” is a moiety that can accept a saccharide from a saccharide donor. A glycosyltransferase can covalently couple a saccharide to a saccharide acceptor. By way of a non-limiting example, G-CSF may be a GalNAc acceptor, and a GalNAc moiety may be covalently coupled to a GalNAc acceptor by way of a GalNAc−transferase.

An oligosaccharide with a “defined size” is one which consists of an identifiable number of monosaccharide units. For example, an oligosaccharide consisting of 10 monosaccharide units is one which may consist of 10 identical monosaccharide units or 5 monosaccharide units of a first identity and 5 monosaccharide units of a second identity. Further, an oligosaccharide of defined size that consists of monosaccharide units of heterogeneous identity may have the monosaccharide units in any order from beginning to end of the oligosaccharide.

An oligosaccharide of “random size” is one which may be synthesized using methods that do not provide oligosaccharide products of defined size. For example, a method of oligosaccharide synthesis may provide oligosaccharides that range from two monosaccharide units to twenty-two saccharide units, including any or all lengths in between.

“Commercial scale” refers to gram scale production of a product saccharide, or glycoprotein, or glycopeptide in a single reaction. In preferred embodiments, commercial scale refers to production of greater than about 50, 75, 80, 90 or 100, 125, 150, 175, or 200 grams.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-NeusAc or 9-O-acetyl-NeuSAc, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

A “method of remodeling a protein, a peptide, a glycoprotein, or a glycopeptide” as used herein, refers to addition of a sugar residue to a protein, a peptide, a glycoprotein, or a glycopeptide using a glycosyltransferase. In a preferred embodiment, the sugar residue is covalently attached to a PEG molecule.

An “unpaired cysteine residue” as used herein, refers to a cysteine residue, which in a correctly folded protein (i.e., a protein with biological activity), does not form a disulfide bind with another cysteine residue.

An “insoluble glycosyltransferase” refers to a glycosyltransferase that is expressed in bacterial inclusion bodies. Insoluble glycosyltransferases are typically solubilized or denatured using e.g., detergents or chaotropic agents or some combination. “Refolding” refers to a process of restoring the structure of a biologically active glycosyltransferase to a glycosyltransferase that has been solubilized or denatured. Thus, a refolding buffer, refers to a buffer that enhances or accelerates refolding of a glycosyltransferase.

A “redox couple” refers to mixtures of reduced and oxidized thiol reagents and include reduced and oxidized glutathione (GSH/GSSG), cysteine/cystine-cysteamine/cystamine, DTT/GSSG, and DTE/GSSG. (See, e.g., Clark, Cur. Op. Biotech. 12:202-207 (2001)).

The term “contacting” is used herein interchangeably with the following: combined with, added to, mixed with, passed over, incubated with, flowed over, etc.

The term “PEG” refers to poly(ethylene glycol). PEG is an exemplary polymer that has been conjugated to peptides. The use of PEG to derivatize peptide therapeutics has been demonstrated to reduce the immunogenicity of the peptides and prolong the clearance time from the circulation. For example, U.S. Pat. No. 4,179,337 (Davis et al.) concerns non-immunogenic peptides, such as enzymes and peptide hormones coupled to polyethylene glycol (PEG) or polypropylene glycol. Between 10 and 100 moles of polymer are used per mole peptide and at least 15% of the physiological activity is maintained.

The term “specific activity” as used herein refers to the catalytic activity of an enzyme, e.g., a recombinant glycosyltransferase fission protein of the present invention, and may be expressed in activity units. As used herein, one activity unit catalyzes the formation of 1 μmol of product per minute at a given temperature (e.g., at 37° C.) and pH value (e.g., at pH 7.5). Thus, 10 units of an enzyme is a catalytic amount of that enzyme where 10 μmol of substrate are converted to 10 μmol of product in one minute at a temperature of, e.g., 37° C. and a pH value of, e.g., 7.5.

“N-linked” oligosaccharides are those oligosaccharides that are linked to a peptide backbone through asparagine, by way of an asparagine-N-acetylglucosamine linkage. N-linked oligosaccharides are also called “N-glycans.” All N-linked oligosaccharides have a common pentasaccharide core of Man₃GlcNAc₂. They differ in the presence of, and in the number of branches (also called antennae) of peripheral sugars such as N-acetylglucosamine, galactose, N-acetylgalactosamine, fucose and sialic acid. Optionally, this structure may also contain a core fucose molecule and/or a xylose molecule.

“O-linked” oligosaccharides are those oligosaccharides that are linked to a peptide backbone through threonine, serine, hydroxyproline, tyrosine, or other hydroxy-containing amino acids.

The term “substantially” in the above definitions of “substantially uniform” generally means at least about 60%, at least about 70%, at least about 80%, or more preferably at least about 90%, and still more preferably at least about 95% of the acceptor substrates for a particular glycosyltransferase are glycosylated.

A “fusion protein” refers to a protein comprising amino acid sequences that are in addition to, in place of, less than, and/or different from the amino acid sequences encoding the original or native full-length protein or subsequences thereof.

A “stem region” with reference to glycosyltransferases refers to a protein domain, or a subsequence thereof, which in the native glycosyltransferases is located adjacent to the trans-membrane domain, and has been reported to function as a retention signal to maintain the glycosyltransferase in the Golgi apparatus and as a site of proteolytic cleavage. Stem regions generally start with the first hydrophilic amino acid following the hydrophobic transmembrane domain and end at the catalytic domain, or in some cases the first cysteine residue following the transmembrane domain. Exemplary stem regions include, but is not limited to, the stem region of eukaryotic ST6GalNAcI, amino acid residues from about 30 to about 207 (see e.g., the murine enzyme), amino acids 35-278 for the human enzyme or amino acids 37-253 for the chicken enzyme; the stem region of mammalian GalNAcT2, amino acid residues from about 71 to about 129 (see e.g., the rat enzyme).

A “catalytic domain” refers to a protein domain, or a subsequence thereof, that catalyzes an enzymatic reaction performed by the enzyme. For example, a catalytic domain of a sialyltransferase will include a subsequence of the sialyltransferase sufficient to transfer a sialic acid residue from a donor to an acceptor saccharide. A catalytic domain can include an entire enzyme, a subsequence thereof, or can include additional amino acid sequences that are not attached to the enzyme, or a subsequence thereof, as found in nature.

The term “isolated” refers to material that is substantially or essentially free from components which interfere with the activity of an enzyme. For a saccharide, protein, or nucleic acid of the invention, the term “isolated” refers to material that is substantially or essentially free from components which normally accompany the material as found in its native state. Typically, an isolated saccharide, protein, or nucleic acid of the invention is at least about 80% pure, usually at least about 90%, and preferably at least about 95% pure as measured by band intensity on a silver stained gel or other method for determining purity. Purity or homogeneity can be indicated by a number of means well known in the art. For example, a protein or nucleic acid in a sample can be resolved by polyacrylamide gel electrophoresis, and then the protein or nucleic acid can be visualized by staining. For certain purposes high resolution of the protein or nucleic acid may be desirable and HPLC or a similar means for purification, for example, may be utilized.

### DESCRIPTION

### I. Isolated Nucleic Acids

### A. Generally

Exemplified herein are various truncation mutants of mammalian ST6GalNAcI and chicken ST6GalNAcI. However, the present invention should not be construed to cover a chicken ST6GalNAcI truncation mutant polypeptide lacking amino acid residues 1-232.

Full-length ST6GalNAcI nucleic acids encode polypeptides that have a domain structure similar to other glycosyltransferases, including an N-terminal signal domain, a transmembrane domain, a stem domain, and an active domain, wherein the active domain may comprise the majority of the amino acid sequence of such polypeptides. As will be understood by one of skill in the art, the presence of domain structure(s) extraneous to the active domain of recombinant ST6GalNAcI polypeptides may have a negative effect on the solubility, stability and activity of the polypeptide in an aqueous or in vitro environment. For example, while not wishing to be bound by any particular theory, the presence of a hydrophobic transmembrane domain on a recombinant ST6GalNAcI polypeptide used in an in vitro reaction mixture may render the polypeptide less soluble than a recombinant ST6GalNAcI polypeptide without a hydryophobic transmembrane domain, and further, may even decrease the enzymatic activity of the polypeptide by affecting or destabilizing the folded structure.

Therefore, it is desirable to produce recombinant ST6GalNAcI nucleic acids that encode ST6GalNAcI that is shorter than full-length ST6GalNAcI, for the purpose of enhancing the activity, stability and/or utility of ST6GalNAcI polypeptides. The present invention provides such modified forms of ST6GalNAcI More particularly, the present invention provides isolated nucleic acids encoding such truncated polypeptides.

Nucleic acids of the present invention encode truncated forms of ST66GalNAcI polypeptides, as described in greater detail elsewhere herein A truncated ST6GalNAcI polypeptide encoded by a nucleic acid of the present invention, also referred to herein as a “truncation mutant,” may be truncated in various ways, as would be understood by the skilled artisan. Examples of truncated polypeptides encoded by a nucleic acid of the present invention include, but are not limited to, a polypeptide lacking a single N-terminal residue, a polypeptide lacking a single C-terminal residue, a polypeptide lacking both an single N-terminal residue and a single C-terminal residue, a polypeptide lacking a contiguous sequence of residues from the N-terminus, a polypeptide lacking a contiguous sequence of residues from the C-terminus, and any combinations thereof.

Therefore, it will be understood, based on the disclosure set forth herein, that truncations of nucleic acids encoding ST6GalNAcI polypeptides may be made for numerous reasons. In one embodiment of the invention, a truncation may be made in order to remove part or all of the nucleic acid sequence encoding the signal peptide domain of an ST6GalNAcI.

In another embodiment of the invention, a truncation may be made in order to remove part or all of a nucleic acid sequence encoding a transmembrane domain of an ST6GalNAcI. By way of a non-limiting example, removal of a part or all of a nucleic acid sequence encoding a transmembrane domain may increase the solubility or stability of the encoded ST6GalNAcI polypeptide and/or may increase the level of expression of the encoded polypeptide.

In yet another embodiment of the invention, a truncation may be made in order to remove part or all of a nucleic acid sequence encoding a stem domain of an ST6GalNAcI. By way of a non-limiting example, removal of a part or all of a nucleic acid sequence encoding a stem domain may increase the solubility or stability of the encoded ST6GalNAcI polypeptide and/or may increase the level of expression of the encoded polypeptide.

The skilled artisan, when armed with the disclosure set forth herein, would understand how to design and create a truncation mutant of ST6GalNAcI as set forth in detail elsewhere herein. In one aspect of the invention, the nucleic acid residue at which a truncation is made may be a highly-conserved residue. In another aspect of the invention, the nucleic acid residue at which a truncation is made may be selected such that the encoded polypeptide has a new N-terminal amino acid residue that will aid in the purification of the expressed polypeptide.

### B. ST6GalNAcI Isolated Nucleic Acids

The present invention features nucleic acids encoding smaller than full-length ST6GalNAcI. That is, the present invention features a nucleic acid encoding a truncated ST6GalNAcI polypeptide, provided the polypeptide expressed by the nucleic acid retains the biological activity of the full-length protein. In one aspect of the invention, a truncated polypeptide is a mammalian truncated ST6GalNAcI polypeptide. In another aspect of the invention, a truncated polypeptide is a human truncated ST6GalNAcI polypeptide. In yet another aspect of the invention, a truncated polypeptide is a mouse truncated ST6GalNAcI polypeptide. In still another aspect of the invention, a truncated polypeptide is a chicken truncated ST6GalNAcI polypeptide.

As would be understood by the skilled artisan, a nucleic acid encoding a full-length ST6GalNAcI may contain a nucleic acid sequence encoding one or more identifyable polypeptide domains in addition to the “active domain,” the domain primarily responsible for the catalytic activity, of ST6GalNAcI. This is because it is known in that art that a full-length ST6GalNAcI polypeptide contains a signal domain, a transmembrane domain, and a stem domain, in addition to an active domain. Accordingly, a nucleic acid encoding a full-length ST6GalNAcI may encode a polypeptide that has a signal domain at the amino-terminus of the polypeptide, followed by a transmembrane domain immediately adjacent to the signal domain, followed by a stem domain that is immediately adjacent to the transmembrane domain, followed by an active domain that extends to the carboxy-terminus of the polypeptide and is located immediately adjacent to the stem domain.

Therefore, in one embodiment, an isolated nucleic acid of the invention may encode a truncated mammalian ST6GalNAcI polypeptide, wherein the truncated ST6GalNAcI polypeptide is lacking all or a portion of the ST6GalNAcI signal domain. In another embodiment, an isolated nucleic acid of the invention may encode a truncated mammalian ST6GalNAcI polypeptide, wherein the truncated ST6GalNAcI polypeptide is lacking the ST6GalNAcI signal domain and all or a portion of the ST6GalNAcI transmembrane domain. In yet another embodiment, a nucleic acid of the invention may encode a truncated mammalian ST6GalNAcI polypeptide, wherein the truncated ST6GalNAcI polypeptide is lacking the ST6GalNAcI signal domain, the ST6GalNAcI transmembrane domain and all or a portion the ST6GalNAcI stem domain

When armed with the disclosure of the present invention, the skilled artisan will know how to make and use these and other such truncation mutants of ST6GalNAcI. In particular, when armed with the disclosure of the present invention, the skilled artisan will know how to make and use isolated nucleic acids encoding truncation mutants of human ST6GalNAcI, mouse ST6GalNAcI and chicken ST6GalNAcI.

The “biological activity of ST6GalNAcI” is the ability to transfer a sialic acid moiety from a sialic acid donor to an acceptor molecule. Full-length human ST6GalNAcI, for example, the sequence of which is set forth in SEQ ID NO: 1, possesses such activity. The “biological activity of a ST6GalNAcI truncated polypeptide” is similarly the ability to transfer a sialic acid moiety from a sialic acid donor to an acceptor molecule. That is, a truncated ST6GalNAcI polypeptide of the present invention can catalyze the same glycosyltransfer reaction as the full-length ST6GalNAcI. By way of a non-limiting example, a truncated human ST6GalNAcI polypeptide encoded by an ST6GalNAcI nucleic acid of the invention has the ability to transfer a sialic acid moiety from a CMP-sialic acid donor to a bovine submaxillary mucin acceptor, wherein such a transfer results in the covalent coupling of a sialic acid moiety to a GalNAc residue on the bovine submaxillary mucin acceptor.

Therefore, a nucleic acid encoding a smaller than full-length, or “truncated,” ST6GalNAcI is included in the present invention provided that the truncated ST6GalNAcI has ST6GalNAcI biological activity.

The methods and compositions of the invention should not be construed to be limited solely to a nucleic acid comprising a ST6GalNAcI truncation mutant as disclosed herein, but rather, should be construed to encompass any nucleic acid encoding a ST6GalNAcI truncated mutant, prepared in accordance with the disclosure herein, either known or unknown, which is capable of catalyzing transfer of a sialic acid to a sialic acid acceptor. Modified nucleic acid sequences, i.e. nucleic acid sequences having sequences that differ from the nucleic acid sequences encoding the naturally-occurring proteins, are also encompassed by methods and compositions of the invention, so long as the modified nucleic acid still encodes a truncated protein having the biological activity of catalyzing the transfer of a sialic acid to a sialic acid acceptor, for example. These modified nucleic acid sequences include modifications caused by point mutations, modifications due to the degeneracy of the genetic code or naturally occurring allelic variants, and further modifications that have been introduced by genetic engineering, i.e., by the hand of man. Thus, the term nucleic acid also specifically includes nucleic acids composed of bases other than the five biologically occurring bases (adenine, guanine, thymine, cytosine and uracil).

The present invention features an isolated nucleic acid comprising a nucleic acid sequence that is at least about 90%; 95%, 97%, 98%, or 99% identical to a nucleic acid sequence set forth in any one of SEQ ID NO:9, SEQ ID NO:11, SEQ ID NO:13, SEQ ID NO: 17, Δ51, SEQ ID NO:21, SEQ ID NO:23, Δ200, SEQ ID NO:27, SEQ ID NO:29, SEQ ID NO:31 and SEQ ID NO:33. The present invention also features an isolated nucleic acid sequence comprising any one of the sequences set forth in SEQ ID NO:9, SEQ ID NO:11, SEQ ID NO:13, SEQ ID NO:17, Δ51, SEQ ID NO:21, SEQ ID NO:23, Δ200, SEQ ID NO:27, SEQ ID NO:29, SEQ ID NO:31 or SEQ ID NO:33, wherein the isolated nucleic acid encodes a truncated ST6GalNAcI polypeptide. The invention further includes an nucleic acid that encodes a truncated ST6GalNAcI polypeptide listed in Table 20.

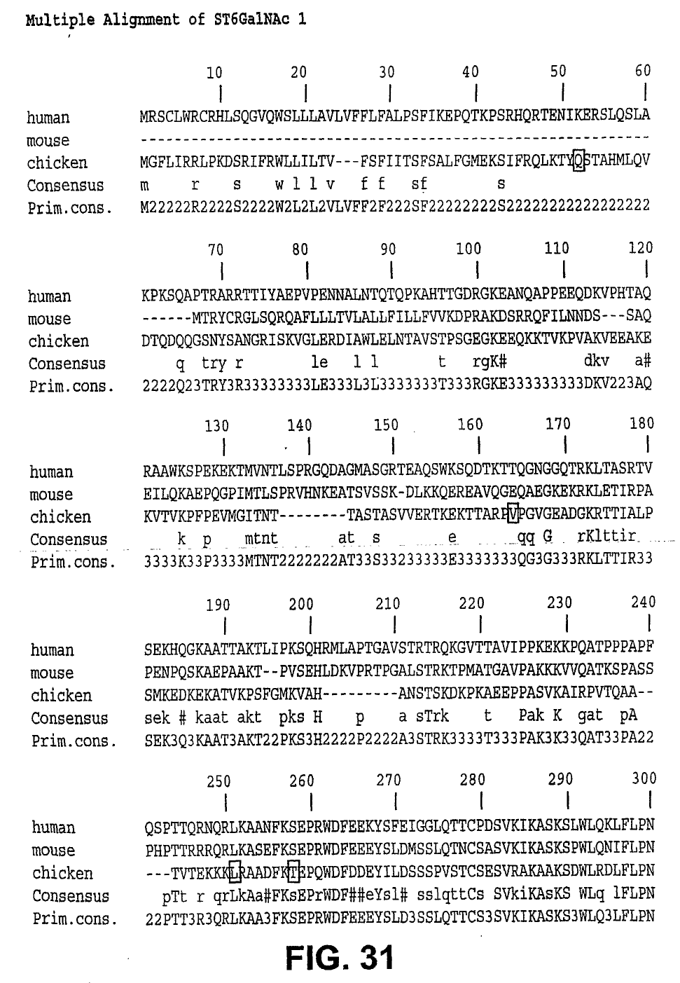

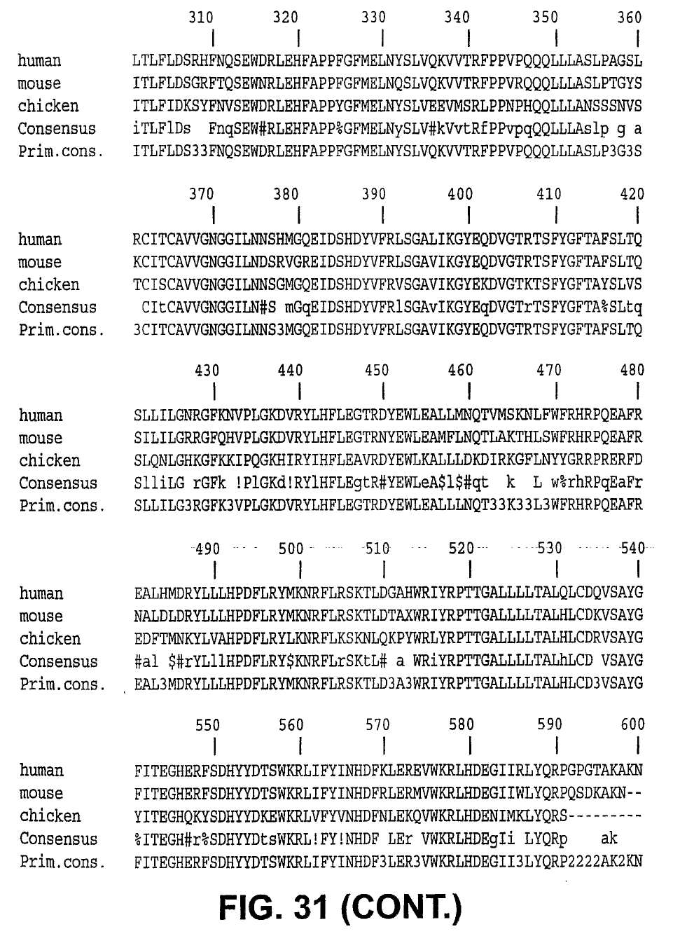

The present invention also encompasses isolated nucleic acid molecules encoding a truncated ST6GalNAcI polypeptide that contains changes in amino acid residues that are not essential for activity. Such polypeptides encoded by an isolated nucleic acid of the invention differ in amino acid sequence from any one of the sequences set forth in SEQ ID NO:10, SEQ ID NO: 12, SEQ ID NO: 14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, Δ225 of the chicken sequence shown in FIG. 31, SEQ ID NO: 18, Δ30 of the mouse sequence shown in FIG. 31, Δ51 of the mouse sequence shown in FIG. 31, SEQ ID NO:22, SEQ ID NO:24 and Δ200 of the mouse sequence shown in FIG. 31; yet retain the biological activity of ST6GalNAcI. By way of a non-limiting example, an isolated nucleic acid of the invention may include a nucleotide sequence encoding a polypeptide having an amino acid sequence that is at least about 90%, 95%, 97%, 98%, or 99% identical to the amino acid sequence of SEQ ID NO: 10. Further, by way of another non-limiting example, an isolated nucleic acid of the invention includes a nucleotide sequence encoding a polypeptide that has an amino acid sequence at least about 90%, 95%, 97%, 98%, or 99% identical to an amino acid sequence set forth in any one of SEQ ID NO:10, SEQ ID NO:12, SEQ ID NO:14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, Δ225 of the chicken sequence shown in FIG. 31, SEQ ID NO: 18, Δ30 of the mouse sequence shown in FIG. 31, Δ51 of the mouse sequence shown in FIG. 31, SEQ ID NO:22, SEQ ID NO:24 and Δ200 of the mouse sequence shown in FIG. 31.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward 1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See, generally, the internet website for the National Center for Biotechnology Information, which is maintained by the National Library of Medicine and the National Institutes of Health.

In another aspect, a nucleic acid useful in the methods and compositions of the present invention and encoding a truncated ST6GalNAcI polypeptide may have at least one nucleotide inserted into the nucleic acid sequence of such a truncated mutant. Alternatively, an additional nucleic acid encoding a truncated ST6GalNAcI polypeptide may have at least one nucleotide deleted from the nucleic acid sequence. Further, a ST6GalNAcI nucleic acid encoding a truncated mutant and useful in the invention may have both a nucleotide insertion and a nucleotide deletion present in a single nucleic acid sequence encoding the truncated polypeptide.

Techniques for introducing changes in nucleotide sequences that are designed to alter the functional properties of the encoded proteins or polypeptides are well known in the art. Such modifications include the deletion, insertion, or substitution of bases, and thus, changes in the amino acid sequence. As is known to one of skill in the art, nucleic acid insertions and/or deletions may be designed into the gene for numerous reasons, including, but not limited to modification of nucleic acid stability, modification of nucleic acid expression levels, modification of expressed polypeptide stability or half-life, modification of expressed polypeptide activity, modification of expressed polypeptide properties and characteristics, and changes in glycosylation pattern. All such modifications to the nucleotide sequences encoding such proteins are encompassed by the present invention.

It is not intended that methods and compositions of the present invention be limited by the nature of the nucleic acid employed. The target nucleic acid encompassed by methods and compositions of the invention may be native or synthesized nucleic acid. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89.

### II. Vectors and Expression Systems

In other related aspects, the invention includes an isolated nucleic acid encoding a truncated ST6GalNAcI polypeptide operably linked to a nucleic acid comprising a promoter/regulatory sequence such that the nucleic acid is preferably capable of directing expression of the polypeptide encoded by the nucleic acid. Thus, the invention encompasses expression vectors and methods for the introduction of exogenous DNA into cells with concomitant expression of the exogenous DNA in those cells, as described, for example, in Sambrook et al. (Third Edition, 2001, Molecular Cloning: A Laboratory Manual, Cold Spring Harbor Laboratory, New York), and in Ausubel et al. (1997, Current Protocols in Molecular Biology, John Wiley & Sons, New York).

Expression of a truncated ST6GalNAcI polypeptide in a cell may be accomplished by generating a plasmid, viral, or other type of vector comprising a nucleic acid encoding the appropriate nucleic acid, wherein the nucleic acid is operably linked to a promoter/regulatory sequence which serves to drive expression of the encoded polypeptide, with or without tag, in cells in which the vector is introduced. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the truncated ST6GalNAcI polypeptide operably linked thereto.

In an expression system useful in the present invention, a nucleic acid encoding a truncated ST6GalNAcI polypeptide may be fused to one or more additional nucleic acids encoding a functional polypeptide. By way of a non-limiting example, an affinity tag coding sequence may be inserted into a nucleic acid vector adjacent to, upstream from, or downstream from a truncated ST6GalNAcI polypeptide coding sequence. As will be understood by one of skill in the art, an affinity tag will typically be inserted into a multiple cloning site in frame with the truncated ST6GalNAcI polypeptide. One of skill in the art will also understand that an affinity tag coding sequence can be used to produce a recombinant fusion protein by concomitantly expressing the affinity tag and truncated ST6GalNAcI polypeptide. The expressed fusion protein can then be isolated, purified, or identified by means of the affinity tag.

Affinity tags useful in the present invention include, but are not limited to, a maltose binding protein, a histidine tag, a Factor IX tag, a glutathione-S-transferase tag, a FLAG-tag, and a starch binding domain tag. Other tags are well known in the art, and the use of such tags in the present invention would be readily understood by the skilled artisan.

As would be understood by one of skill in the art, a vector comprising a truncated ST6GalNAcI polypeptide of the present invention may be used to express the truncated polypeptide as either a non-fusion or as a fusion protein. Selection of any particular plasmid vector or other DNA vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding either a truncated ST6GalNAcI polypeptide. Such technology is well known in the art and is described, for example, in Sambrook et al. (Third Edition, 2001, Molecular Cloning: A Laboratory Manual, Cold Spring Harbor Laboratory, New York), and in Ausubel et al. (1997, Current Protocols in Molecular Biology, John Wiley & Sons, New York). By way of a non-limiting example, a vector useful in one embodiment of the present invention is based on the pcWori+ vector (Muchmore et. al., 1987, Meth. Enzymol. 177:44-73).

The invention thus includes a vector comprising an isolated nucleic acid encoding a truncated ST6GalNAcI polypeptide. The incorporation of a nucleic acid into a vector and the choice of vectors is well-known in the art as described in, for example, Sambrook et al. (Third Edition, 2001, Molecular Cloning: A Laboratory Manual, Cold Spring Harbor Laboratory, New York), and in Ausubel et al. (1997, Current Protocols in Molecular Biology, John Wiley & Sons, New York).

In an aspect of the invention, an isolated nucleic acid encoding a truncated ST6GalNAcI polypeptide is integrated into the genome of a host cell in conjunction with a nucleic acid encoding a truncated ST6GalNAcI polypeptide. In another aspect of the invention, a cell is transiently transfected with an isolated nucleic acid encoding a truncated ST6GalNAcI polypeptide. In another aspect of the invention, a cell is stably transfected with an isolated nucleic acid encoding a truncated ST6GalNAcI polypeptide.

For the purpose of inserting an isolated nucleic acid into a cell, one of skill in the art would also understand that the methods available and the methods required to introduce an isolated nucleic acid of the invention into a host cell vary and depend upon the choice of host cell. Suitable methods of introducing an isolated nucleic acid into a host cell are well-known in the art. Other suitable methods for transforming or transfecting host cells may include, but are not limited to, those found in Sambrook, et al. (Molecular Cloning: A Laboratory Manual. 3rd ed., Cold Spring Harbor Laboratory, Cold Spring Harbor Laboratory Press, Cold Spring Harbor, N.Y., 2001), and other such laboratory manuals.

A nucleic acid encoding a truncated ST6GalNAcI polypeptide may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

The present invention also features a recombinant bacterial host cell comprising, inter alia, a nucleic acid vector as described elsewhere herein. In one aspect, the recombinant cell is transformed with a vector of the present invention. The transformed vector need not be integrated into the cell genome nor does it need to be expressed in the cell. However, the transformed vector will be capable of being expressed in the cell. In one aspect of the invention, a B. subtilis cell is used for transformation of a vector of the present invention and expression of protein therefrom. In another aspect of the invention, E. coli is used for transformation of a vector of the present invention and expression of protein therefrom. In another aspect of the invention, a K-12 strain of E. coli is useful for expression of protein from a vector of the present invention. Strains of E. coli useful in the present invention include, but are not limited to, JM83, JM101, JM103, JM109, W3110, chi1776, and JA221.

It will be understood that a host cell useful in the present invention will be capable of growth and culture on a small scale, medium scale, or a large scale. For example, a host cell of the invention is useful for testing the expression of a protein from a vector of the invention equally as much as it is useful for large scale production of a reagent or therapeutic protein product. Techniques useful in culturing host cells and expressing protein from a vector contained therein are well known in the art and will therefore not be listed herein.

A host cell useful in methods of the present invention, as described above, may be prepared according to various methods, as would be understood by the skilled artisan when amend with the disclosure set forth herein. In one aspect, a host cell of the present invention may be transformed with a vector of the present invention to produce a transformed host cell of the invention. Transformation, as known to the skilled artisan, includes the process of inserting a nucleic acid vector into a host cell, such that the host cell containing the nucleic acid vector remains viable. Such transformation of nucleic acid into a bacterial cell is useful for purposes including, but not limited to, creation of a stably-transformed host cell, making a biological deposit, propagating the vector-containing host cell, propagating the vector-containing host cell for the production and isolation of additional vector, expression of target protein encoded by vector, and the like.

Methods of transforming a cell with a vector are numerous and well-known in the art, and will therefore not be listed here. By way of a non-limiting example, a competent bacterial cell of the invention may be transformed by a vector of the invention using electroporation. Methods of making bacterial cells “competent” are well-known in the art, and typically involve preparation of the bacterial cells so that the cells take up exogenous DNA. Similarly, methods of electroporation are known in the art, and detailed descriptions of such methods may be found, for example, in Sambrook et al. (1989, supra). The transformation of a competent cell with vector DNA may be also accomplished using chemical-based methods. One example of a well-known chemical-based method of bacterial transformation is described by Inoue, et al. (1990, Gene 96:23-28). Other methods of transformation will be known to the skilled artisan.

A transformed host cell of the present invention may be used to express a truncated ST6GalNAcI polypeptide of the present invention. In an embodiment of the invention, a transformed host cell contains a vector of the invention, which contains therein a nucleic acid sequence encoding an truncated polypeptide of the invention. The truncated polypeptide is expressed using any expression method known in the art (for example, IPTG). The expressed truncated polypeptide may be contained within the host cell, or it may be secreted from the host cell into the growth medium.

Methods for isolating an expressed polypeptide are well-known in the art, and the skilled artisan will know how to determine the best method for isolation of an expressed polypeptide based on the characteristics of any given host cell expression system. By way of a non-limiting example, an expressed polypeptide that is secreted from a host cell may be isolated from the growth medium. Isolation of a polypeptide from a growth medium may include removal of bacterial cells and cellular debris. By way of another non-limiting example, an expressed polypeptide that is contained within a host cell may be isolated from the host cell. Isolation of such an “intracellular” expressed polypeptide may include disruption of the host cell and removal of cellular debris from the resultant mixture. These methods are not intended to be exclusive representations of the present invention, but rather, are merely for the purposes of illustration of various applications of the present invention.

Purification of a truncated polypeptide expressed in accordance with the present invention may be effected by any means known in the art. The skilled artisan will know how to determine the best method for the purification of a polypeptide expressed in accordance with the present invention. A purification method will be chosen by the skilled artisan based on factors such as, but not limited to, the expression host, the contents of the crude extract of the polypeptide, the size of the polypeptide, the properties of the polypeptide, the desired end product of the polypeptide purification process, and the subsequent use of the end product of the polypeptide purification process

In an embodiment of the invention, isolation or purification of a truncated polypeptide expressed in accordance with the present invention may not be desired. In an aspect of the present invention, an expressed polypeptide may be stored or transported inside the bacterial host cell in which the polypeptide was expressed. In another aspect of the invention, an expressed polypeptide may be used in a crude lysate form, which is produced by lysis of a host cell in which the polypeptide was expressed. In yet another embodiment of the invention, an expressed polypeptide may be partially isolated or partially purified according to any of the methods set forth or described herein. The skilled artisan will know when it is not desirable to isolate or purify a polypeptide of the invention, and will be familiar with the techniques available for the use and preparation of such polypeptides.

When armed with the disclosure set forth herein, the skilled artisan would also know how to prepare a eukaryotic host cell of the invention. As set forth elsewhere herein, and as would be known to one of skill in the art based on the disclosure provided herein, an isolated nucleic acid encoding a truncated ST6GalNAcI polypeptide may be introduced into a eukaryotic host cell, for example, using a lentivirus-based genomic integration or plasmid-based transfection (Sambrook et al., Third Edition, Molecular Cloning: A Laboratory Manual, Cold Spring Harbor Laboratory, New York (2001)). In one embodiment of the invention, a eukaryotic host cell is a fungal cell. Fungal cells useful as eukaryotic host cells of the invention include, but should not be limited to, strains such as A. niger and P. lucknowensa.

In another embodiment, a nucleic acid encoding a truncated polypeptide of the invention is cloned into a lentiviral vector containing a specific promoter sequence for expression of the truncated polypeptide. The truncated polypeptide-containing lentiviral vector is then used to transfect a host cell for expression of the truncated polypeptide. Methods of making and using lentiviral vectors, such as those useful in the present invention, are well-known in the art and are not described further herein.

In yet another embodiment, a nucleic acid encoding a truncated polypeptide of the invention is introduced into a host cell using a viral expression system. Viral expression systems are well-known in the art, and will not be described in detail herein. In one aspect of the invention, a viral expression system is a mammalian viral expression system. In another aspect of the invention, a viral expression system is a baculovirus expression system. Such viral expression systems are typically commercially available from numerous vendors.

The skilled artisan will know how to use a host cell-vector expression system for the expression of a truncated polypeptide of the invention. Appropriate cloning and expression vectors for use with eukaryotic hosts arc described by Sambrook, et al., in Molecular Cloning: A Laboratory Manual, Third Edition, Cold Spring Harbor, N.Y. (2001), the disclosure of which is hereby incorporated in its entirety by reference.

Insect cells can also be used for expression of a truncated polypeptide of the present invention. In an aspect of the invention, Sf9, Sf9⁺, Sf21, High Five™ or Drosophila Schneider S2 cells can be used. In yet another aspect of the invention, a baculovirus, or a baculovirus/insect cell expression system can be used to express a truncated polypeptide of the invention using a pAcGP67, pFastBac, pMelBac, or pIZ vector and a polyhedrin, p10, or OpIE3 actin promoter. In another aspect of the invention, a Drosophila expression system can be used with a pMT or pAC5 vector and an MT or Ac5 promoter.

A truncated ST6GalNAcI polypeptide of the invention of the invention can also be expressed in mammalian cells. In an aspect of the invention, 294, HeLa, HEK, NSO, Chinese hamster ovary (CHO), Jurkat, or COS cells can be used to express a truncated polypeptide of the invention. In the case of a mammalian cell expression of a truncated polypeptide, a suitable vector such as pT-Rex, pSecTag2, pBudCE4.1, or pcDNA/His Max vector can be used, along with, for example, a CMV promoter. As will be understood by the skilled artisan, the choice of promoter, as well as methods and strategies for introducing one or more promoters into a host cell used for expressing a truncated ST6GalNAcI polypeptide of the invention are well-known in the art, and will vary depending upon the host cell and expression system used.

Various mammalian cell culture systems can be employed to express recombinant protein. Non-limiting examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell tines. Mammalian expression vectors may comprise an origin of replication, a suitable promoter and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements.

The methods available and the methods required to introduce any isolated nucleic acid of the invention into a host cell vary and depend upon the choice of the host cell, as would be understood by one of skill in the art. Suitable methods of introducing an isolated nucleic acid into a host cell are well-known in the art. By way of a non-limiting example, vector DNA can be introduced into a eukaryotic cell using conventional transfection techniques. As used herein, the term “transfection” refers to a variety of art-recognized techniques for introducing foreign nucleic acid (e.g., DNA) into a host cell, including, DEAE-dextran-mediated transfection, lipofection, or electroporation. Suitable methods for transforming or transfecting host cells can be found in Sambrook, et al. (Molecular Cloning: A Laboratory Manual. 3nd ed., Cold Spring Harbor Laboratory, Cold Spring Harbor Laboratory Press, Cold Spring Harbor, N.Y., 2001), and other such laboratory manuals.

For example, for stable transfection of mammalian cells, it is known that, depending upon the expression vector and transfection technique used, only a small fraction of cells may integrate the foreign DNA into their genome. In order to identify and select these transformants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding a truncated polypeptide of the invention or can be introduced on a separate vector. Cells stably transfected with the introduced nucleic acid can be identified by drug selection (e.g., cells that have incorporated the selectable marker gene will survive, while the other cells die).

### III. Polypeptides

A truncated polypeptide of the present invention may be truncated in various ways, as would be known and understood by the skilled artisan, when armed with the disclosure set forth herein. Examples of truncated polypeptides of the present invention include, but are not limited to, a polypeptide lacking a single N-terminal residue, a polypeptide lacking a single C-terminal residue, a polypeptide lacking both an single N-terminal residue and a single C-terminal residue, a polypeptide lacking a contiguous sequence of residues from the N-terminus, a polypeptide lacking a contiguous sequence of residues from the C-terminus, and any such combinations thereof.

As would be understood by the skilled artisan, a lull-length human ST6GalNAcI polypeptide may contain one or more identifyable polypeptide domains in addition to the “active domain,” the domain primarily responsible for the catalytic activity, of ST6GalNAcI. This is because it is known in that art that a full-length ST6GalNAcI polypeptide contains a signal domain, a transmembrane domain, and a stem domain, in addition to an active domain. Accordingly, a full-length ST6GalNAcI may have a signal domain at the amino-terminus of the polypeptide, followed by a transmembrane domain immediately adjacent to the signal domain, followed by a stem domain that is immediately adjacent to the transmembrane domain, followed by an active domain that extends to the carboxy-terminus of the polypeptide and is located immediately adjacent to the stem domain.

Therefore, in one embodiment, a ST6GalNAcI polypeptide of the invention is a truncated mammalian ST6GalNAcI polypeptide lacking all or a portion of the ST6GalNAcI signal domain. In another embodiment, a ST6GalNAcI polypeptide of the invention is a truncated mammalian ST6GalNAcI polypeptide lacking the ST6GalNAcI signal domain and all or a portion of the ST6GalNAcI transmembrane domain. In yet another embodiment, a ST6GalNAcI polypeptide of the invention is a truncated mammalian ST6GalNAcI polypeptide lacking the ST6GalNAcI signal domain, the ST6GalNAcI transmembrane domain and all or a portion the ST6GalNAcI stem domain. When armed with the disclosure of the present invention, the skilled artisan will know how to make and use these and other such truncation mutants of human ST6GalNAcI.

The size and identity of a truncated ST6GalNAcI mutant of the present invention is based on the point at which the full-length polypeptide is truncated. By way of a non-limiting example, a “Δ35 human truncated ST6GalNAcI” mutant of the invention refers to a truncated ST6GalNAcI polypeptide of the invention in which amino acids 1 through 35, counting from the N-terminus of the full-length polypeptide, are deleted from the polypeptide. Therefore, the N-terminus of the Δ35 human truncated ST6GalNAcI mutant begins with the amino acid residue that would be referred to as “amino acid 36” of the full-length polypeptide. This nomenclature applies to all truncated ST6GalNAcI polypeptides of the invention, including, but not limited to those derived from mammalian ST6GalNAcI, human ST6GalNAcI, mouse ST6GalNAcI and chicken ST6GalNAcI Where specific deletions are indicated, the deletions are determined using the full length ST6GalNAcI sequence from chicken, mouse, or human shown in FIG. 31. Preferred embodiments of such deletions are shown, e.g., in Table 20. In some embodiments, the truncated ST6GalNAcI mutant is selected from the following. For human truncated ST6GalNAcI mutants (using the two possible names for a single mutant): Δ35 or K36, Δ124 or K125, Δ257 or S258, Δ72 or T73, Δ109 or E110, Δ133 or M134, Δ170 or T171, Δ232 or Δ233 and Δ272 or G273. For chicken truncated ST6GalNAcI mutants (using the two possible names for a single mutant): Δ48 or Q49, Δ152 or V153, Δ225 or L226, Δ226 or R227, Δ231 or K233 and Δ232 or T233. For mouse truncated ST6GalNAcI mutants (using the two possible names for a single mutant): Δ30 or K31, Δ31 or D32, Δ51 or E52, Δ126 or S127, Δ185 or S186, and Δ200 or S201.

The present invention therefore also includes an isolated polypeptide comprising a truncated ST6GalNAcI polypeptide. Preferably, an isolated truncated ST6GalNAcI polypeptide of the present invention has at least about 90% identity to a polypeptide having the amino acid sequence of any one of the sequences set forth in SEQ ID NO: 10, SEQ ID NO: 12, SEQ ID NO: 14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ-ID NO:32, Δ225 of the chicken sequence shown in FIG. 31, SEQ ID NO: 18, Δ30 of the mouse sequence shown in FIG. 31, Δ51 of the mouse sequence shown in FIG. 31, SEQ ID NO:22, SEQ ID NO:24 and Δ200 of the mouse sequence shown in FIG. 31. More preferably, the isolated polypeptide is about 95% identical, and even more preferably, about 98% identical, still more preferably, about 99% identical, and most preferably, the isolated polypeptide comprising a truncated ST6GalNAcI polypeptide is identical to the polypeptide set forth in one of SEQ ID NO: 10, SEQ ID NO: 12, SEQ ID NO:14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, Δ225 of the chicken sequence shown in FIG. 31, SEQ ID NO: 18, Δ30 of the mouse sequence shown in FIG. 31, Δ51 of the mouse sequence shown in FIG. 31, SEQ ID NO:22, SEQ ID NO:24 and Δ200 of the mouse sequence shown in FIG. 31.

The present invention also provides for analogs of polypeptides which comprise a truncated ST6GalNAcI polypeptide as disclosed herein. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Fragments of a truncated ST6GalNAcI polypeptide of the invention are included in the present invention, provided the fragment possesses the biological activity of the full-length polypeptide. That is, a truncated ST6GalNAcI polypeptide of the present invention can catalyze the same glycosyltransfer reaction as the full-length ST6GalNAcI. By way of a non-limiting example, a truncated human ST6GalNAcI polypeptide of the invention has the ability to transfer a sialic acid moiety from a CMP-sialic acid donor to a bovine submaxillary mucin acceptor, wherein such a transfer results in the covalent coupling of a sialic acid moiety to a GalNAc residue on the bovine submaxillary mucin acceptor. Therefore, a smaller than full-length, or “truncated,” ST6GalNAcI is included in the present invention provided that the truncated ST6GalNAcI has ST6GalNAcI biological activity.

In another aspect of the present invention, compositions comprising an isolated truncated ST6GalNAcI polypeptide as described herein may include highly purified truncated ST6GalNAcI polypeptides. Alternatively, compositions comprising truncated ST6GalNAcI polypeptides may include cell lysates prepared from the cells used to express the particular truncated ST6GalNAcI polypeptides. Further, truncated ST6GalNAcI polypeptides of the present invention may be expressed in one of any number of cells suitable for expression of polypeptides, such cells being well-known to one of skill in the art, as described in detail elsewhere herein.

It will be appreciated that all above descriptions of a truncated ST6GalNAcI polypeptide applies equally to truncated ST6GalNAcI polypeptides of the invention from any source, including, but not limited to mammalian ST6GalNAcI, human ST6GalNAcI, mouse ST6GalNAcI, and chicken ST6GalNAcI.

Substantially pure protein isolated and obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego). In a preferred embodiment, the truncated ST6GalNAcI polypeptides of the invention are fused to a purification tag, e.g. a maltose binding domain (MBD) tag or a starch binding domain (SBD) tag. Such truncated ST6GalNAcI fusion proteins can be purified by passage through a column that specifically binds to the purification tag, e.g., MBD or SBD proteins can be purified on a cyclodextrin column. In a further embodiment, a truncated ST6GalNAcI fusion proteins comprising a purification tag, such as, e.g., an MBD or SBD tag, are immobilized on a column that specifically binds to the purification tag and substrates, e.g., a sialic acid donor or PEGylated-sialic acid donor and a glycoprotein or glycopeptide comprising an O-linked glycylation site are passed through the column under conditions that facilitate transfer of sialic acid from a donor, e.g., CMP-sialic acid or CMP-PEGylated-sialic acid, to a glycoprotein or glycopeptide acceptor, and thus production of a sialylated glycoprotein or sialylated glycopeptide.

### III. Methods

The present invention features a method of expressing a truncated polypeptide. Polypeptides which can be expressed according to the methods of the present invention include a truncated ST6GalNAcI polypeptide. More preferably, polypeptides which can be expressed according to the methods of the present invention include, but are not limited to, a truncated human ST6GalNAcI polypeptide, a truncated mouse ST6GalNAcI polypeptide, and a truncated chicken ST6GalNAcI polypeptide. In a preferred embodiment, a polypeptide which can be expressed according to the methods of the present invention is a polypeptide comprising any one of the polypeptide sequences set forth in SEQ ID NO: 10, SEQ ID NO: 12, SEQ ID NO: 14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, Δ225 of the chicken sequence shown in FIG. 31, SEQ ID NO: 18, Δ30 of the mouse sequence shown in FIG. 31, Δ51 of the mouse sequence shown in FIG. 31, SEQ ID NO:22, SEQ ID NO:24 and Δ200 of the mouse sequence shown in FIG. 31.

In one embodiment, the present invention features a method of expressing a truncated ST6GalNAcI polypeptide encoded by an isolated nucleic acid of the invention, as described elsewhere herein, wherein the expressed truncated ST6GalNAcI polypeptide has the property of catalyzing the transfer of a sialic acid moiety to an acceptor moiety. In one aspect of the invention, a method of expressing a truncated ST6GalNAcI polypeptide includes the steps of cloning an isolated nucleic acid of the invention into an expression vector, inserting the expression vector construct into a host cell, and expressing a truncated ST6GalNAcI polypeptide therefrom.

Methods of expression of polypeptides, as well as construction of expression systems and recombinant host cells for expression of polypeptides, are discussed in extensive detail elsewhere herein. Methods of expression of a truncated polypeptide of the present invention will be understood to include, but not to be limited to, all such methods as described herein. In some expression systems, the truncated ST6GalNAcI polypeptides of the invention are expressed as insoluble proteins, e.g., in an inclusion protein in a bacterial host cell. Methods of refolding insoluble glycosyltransferases, including ST6GalNAcI polypeptides, are disclosed in U.S. Provisional Patent Application Ser. No. 60/542,210, filed Feb. 4, 2004; U.S. Provisional Patent Application Serial No: 60/599,406, filed Aug. 6, 2004; U.S. Provisional Patent Application Ser. No. 60/627,406, filed Nov. 12, 2004; and International Patent Application No. PCT/US05/03856, filed Feb. 4, 2005; each of which are herein incorporated by reference for all purposes.

The present invention also features a method of catalyzing a glycosyltransferase reaction between a glycosyl donor and a glycosyl acceptor. In one embodiment, the invention features a method catalyzing the transfer of a sialic acid moiety to an acceptor moiety, wherein the sialyltransfer reaction is carried out by incubating a truncated ST6GalNAcI polypeptide of the invention with a sialic acid donor moiety and an acceptor moiety. In one aspect, a truncated ST6GalNAcI polypeptide of the invention mediates the covalent linkage of a sialic acid moiety to an acceptor moiety, thereby catalyzing the transfer of a sialic acid moiety to an acceptor moiety.

In an embodiment of the invention, a truncated ST6GalNAcI polypeptide useful in a glycosyltransfer reaction is a truncated human ST6GalNAcI polypeptide. In another embodiment, a truncated ST6GalNAcI polypeptide useful in a glycosyltransfer reaction is a truncated chicken ST6GalNAcI polypeptide. In a preferred embodiment, a truncated ST6GalNAcI polypeptide useful in a glycosyltransfer reaction is a polypeptide comprising any one of the polypeptide sequences set forth in SEQ ID NO: 10, SEQ ID NO: 12, SEQ ID NO:14, SEQ ID NO:28, SEQ ID NO:30, SEQ ID NO:32, or any of the human truncated ST6GalNAcI polypeptides listed in Table 20.

By way of a non-limiting example, a method of catalyzing the transfer of a sialic acid moiety to an acceptor moiety includes the steps of incubating a truncated human ST6GalNAcI polypeptide with a cytidinemonophosphate-sialic acid (CMP-NAN) sialic acid donor and an asialo bovine submaxillary mucin acceptor moiety, wherein the truncated human ST6GalNAcI polypeptide mediates the transfer of a sialic acid moiety from CMP-NAN to the bovine submaxillary mucin acceptor.

Therefore, in one embodiment, the present invention also features a polypeptide acceptor moiety. In one embodiment of the invention, a polypeptide acceptor moiety is a human growth hormone. In another embodiment, a polypeptide acceptor moiety is an erythropoietin. In yet another embodiment, a polypeptide acceptor moiety is an interferon-alpha. In another embodiment, a polypeptide acceptor moiety is an interferon-beta. In another embodiment of the invention, a polypeptide acceptor moiety is an interferon-gamma. In still another embodiment of the invention, a polypeptide acceptor moiety is a lysosomal hydrolase. In another embodiment, a polypeptide acceptor moiety is a blood factor polypeptide. In still another embodiment, a polypeptide acceptor moiety is an anti-tumor necrosis factor-alpha. In another embodiment of the invention, a polypeptide acceptor moiety is follicle stimulating hormone. In yet another embodiment of the invention, a polypeptide acceptor moiety is a glucagon-like peptide.

In one embodiment, the present invention also features a method of transferring a sialic acid-polyethyleneglycol conjugate (SA-PEG) to an acceptor molecule. In one aspect, an acceptor molecule is a polypeptide. In another aspect, an acceptor molecule is a glycopeptide. Compositions and methods useful for designing, producing and transferring a SA-PEG conjugate to an acceptor molecule are discussed at length in International (PCT) Patent Application No. WO03/031464 (PCT/US02/32263) and U.S. Patent Application No 2004/0063911, each of which is incorporated herein by reference in its entirety.

Methods of assaying for glycosyltransferase activity are well-known in the art. Various assays for detecting glycosyltransferases which can be used in accordance with the invention have been published. The following are illustrative, but should not be considered limiting, of those assays useful for detecting glycosyltransferase activity. Furukawa et al (1985, Biochem. J., 227:573-582) describe a borate-impregnated paper electrophoresis assay and a fluorescence assay Roth et al (1983, Exp'l Cell Research 143:217-225) describe application of the borate assay to glucuronyl transferases, previously assayed calorimetrically. Benau et al (1990, J. Histochem. Cytochem., 38:23-30) describe a histochemical assay based on the reduction, by NADH, of diazonium salts. See also U.S. Pat. No. 6,284,493 of Roth, incorporated herein by reference.

### EXPERIMENTAL EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

### Example 1

### Molecular Cloning of Mouse GalNAc α2,6-Sialyltransferase (ST6GalNAcI) into the MBP-pCWin2 Vector

The cloning and expression of five N-terminal amino acid truncated GalNAc α2, 6-Sialyltransferase (ST6GalNAcI) genes into the pCWin2 MBP fusion tag expression vector was conducted as described herein. Also described herein is the generation of five different amino-terminal truncations of the ST6GalNAcI gene fused to Maltose binding protein (MBP) in the pCWin2-MBP vector. Generation of JM109 cells transformed with these constructs and the subsequent induction of protein expression in these transformants is presented. All five fusion proteins are expressed at varying levels upon induction with IPTG.

Template DNA (pTS103) was used for amplification of mouse ST6GalNAcI. Primers were designed to clone mouse ST6GalNAcI gene using the following sequences for five N-terminal truncated forms of mouse ST6GalNAcI, including Δ31, Δ51, Δ126, Δ185 and Δ200. The primers used were as follows:

D32-HindIII-5′-taatataagottgatccaagggcaaaagattc-3′ (SEQ ID NO:43), E52-BamHI-5′-taataaggatcogagattctgcaa aaggctga-3 (SEQ ID NO:44), S127-BamHI-5′-taatatggatcctcagaacacctggacaaa gt-3′(SEQ ID NO:45), S186-BamHI-5′-taatatggatcctctgagcctcggtgggattt-3(SEQ ID NO:46), S201-13BamHI-5′-taataaggatccagcagcctgcagacgaactg-3(SEQ ID NO-47), and M-XhoI-5′-tag cgc ctc gag tca gtt ctt tgc ttt gtc act ttg-3′(SEQ ID NO):48). A PCR reaction was conducted in autoclaved 500 μl reaction tubes for amplification of various ST6GalNAcI genes.

TABLE 1
PCR reaction parameters for mouse ST6GalNAcI truncation mutants.

| | Reaction tubes | | | | | | | | | |
|---|---|---|---|---|---|---|---|---|---|---|
| Reagents | D32 | | E52 | | S127 | | S186 | | S201 | |
| 10X Herculase Buffer | 5 | μl | 5 | μl | 5 | μl | 5 | μl | 5 | μl |
| 25 mM MgCl2 | 1 | μl | 1 | μl | 1 | μl | 1 | μl | 1 | μl |
| 10 mM dNTP | 1 | μl | 1 | μl | 1 | μl | 1 | μl | 1 | μl |
| Forward primer 10 pmol/μl | 4 | μlA | 4 | μlB | 4 | μlC | 4 | μlD | 4 | μlE |
| Reverse primer 10 pmol/μl | 4 | μlF | 4 | μlF | 4 | μlF | 4 | μlF | 4 | μlF |
| Nuclease free water | 31 | μl | 31 | μl | 31 | μl | 31 | μl | 31 | μl |
| Template (pTs 103) 12.4 ng | 3 | μl | 3 | μl | 3 | μl | 3 | μl | 3 | μl |
| Herculase polymerase | 1 | μl | 1 | μl | 1 | μl | 1 | μl | 1 | μl |

| | | | | |
|---|---|---|---|---|
| Lid temperature | 105° C. | | | |
| STEP 1 | 92° C. | 45 Seconds | | |
| STEP 2 | 61° C. | 60 seconds | | |
| STEP 3 | 72° C. | 3.0 Minutes. | STEP 1, 2 and 3 | 30 Cycles. |
| STEP 4 | 92° C. | 45 Seconds | | |
| STEP 5 | 61° C. | 60 Seconds | | |
| STEP 6 | 72° C. | 10 Minutes. | STEP 4, 5 and 6 | 4 Cycles. |
| STEP 7 | 4° C. | PAUSE | | |

The results of the PCR reaction were visualized using 0.8% agarose/TAE gels. The ST6GalNAcI gene was identified at about 1.5 Kb. DNA was extracted from the get using Amicon Ultra free DA filters and purified using Microcon YM-100 filters, according to manufacturer's instructions (Millipore, Bellerica, Mass.).

A DNA band around 1.5 Kb in the 0.8% agarose gel was identified using a UV transilluminator. A gel slice containing the DNA was excised from the gel. Using an Amicon Ultra free DA filter Millipore, Bellerica, Mass.), the gel slice was placed in a gel nebulizer and the device sealed with the cap attached to the vial. The assembled device was centrifuged for 10 minutes at 5000×g. The extruded DNA passed through the microporous membrane in the sample filter cup and was collected in the filtrate vial. Purified DNA in the vial was transferred into a sample reservoir of a Microcon YM-100 unit (Millipore, Bellerica, Mass.) and centrifuged at 200 rpm for 12 minutes. The transferred DNA was collected.

Restriction enzyme digestion of concentrated DNA from the PCR reaction was conducted in a 1.5 ml tube by adding 6.0 μl of purified PCR product, 2.5 μl of 10× Bam HI buffer, 2.5 μl of 10×BSA, 1.5 pt of Bam HI enzyme, 1.5 μl XhoI enzyme, and 11.0 μl nuclease free water. Reactions were incubated for 1.5 hours at 37 o C and placed on ice for 5 minutes. MBP-pCWin2 vector DNA was digested in a 1.5 ml tube by adding 6.0 μl vector DNA (MBP-pCWin2), 2.5 μl 10× Bam HI buffer, 2.5 μl 10×BSA, 1.5 μl BamHI enzyme, 1.5 μL XhoI enzyme, and 11.0 μl nuclease free water. The digestion reaction was analyzed by electrophoresis on 0.8% agarose/TAE gels. Gels were loaded with digestion mixtures containing 2 μl of loading dye and 10 μl of digested DNA. DNA around 1-5 Kb was extracted from the gel using the Amicon Ultra free DA protocol and purified using Microcon YM-100 according to manufacturer's instructions (Millipore, Bellerica, Mass.).

In autoclaved 0.5 ml tubes, the following BamHI/XhoI digested DNA was added in order to ligate the insert into the vector:

TABLE 2
Ligation reactions for mouse ST6GalNAcI truncation mutants.

| HindIII/XhoI digested DNA | 1 | 2 | 3 | 4 | 5 |
|---|---|---|---|---|---|
| D32 | 11.5 | — | — | — | — |
| E52 | — | 11.5 | — | — | — |
| S127 | — | — | 11.5 | — | — |
| S186 | — | — | — | 11.5 | — |
| S201 | — | — | — | — | 11.5 |
| μl Bam HI/XhoI digested | 1.5 | 1.5 | 1.5 | 1.5 | 1.5 |
| MBP-pCWIn2 | | | | | |
| μl 10× ligation buffer | 1.5 | 1.5 | 1.5 | 1.5 | 1.5 |
| μl T4 DNA ligase | 0.5 | 0.5 | 0.5 | 0.5 | 0.5 |

Reaction mixtures were incubated at 4° C. overnight.

To each of five pre-chilled 2 mm gap cuvettes numbered 1, 2, 3, 4 and 5 was added 2.0 μl of the ligation reactions listed in Table 2. Mixtures were added to corresponding cuvettes including 50 μl of thawed (on ice) DH5α electrocompetent cells. The mixture was subject to electroporation at 2.5 KV, R5 resistance and 129 OHMS. SOC media (1 ml) was added to each reaction mixture, which was then incubated at 37 o C for one hour with shaking at 225 RPM. 100 μl of each transformation reaction was plated onto LB (50 μg/ml) Kanr plates and incubated at 37oC overnight.

For positive clone screening, four transformant colonies were selected from each construct and were inoculated into 5.0 ml of LB broth containing 10 μg/ml of Kanamycin and grown at 37o C for 5 hours, with shaking (225 rpm). DNA was isolated using a QIA prep Spin Miniprep Kit according to manufacturer's instructions (Qiagen, Valencia, Calif., Valencia, Calif.). Plasmid DNA was prepared with both BamHI/XhoI as described previously. The digestion reactions then were then analyzed on 0.8% agarose/TAE gels.

DNA from colonies #1 through #4, construct DH5α/MBP-pCWin2-ST6GalNAcI (D32, E52, S127, S186, S201, corresponding to Δ31, Δ51, Δ126, Δ185, and Δ200, respectively), was double digested using restriction enzymes NdeI and HindIII as set forth in Table 3 in order to isolate MBP-ST6GalNAcI fragments.

TABLE 3
Diagnostic conditions for ST6GalNAcI truncation mutant DNA isolates.
In 0.5 ml autoclaved tubes:

| | |
|---|---|
| 6.0 μl | DNA from each mutant DH5α/pCWin2-MBP-ST6GalNAcI |
| 2.5 μl | 10X NEB4 Buffer |
| 2.5 μl | 10X BSA |
| 1.5 μl | NdeI enzyme |
| 1.5 μl | XhoI enzyme |
| 11.0 μl | Nuclease free water |

Reactions were incubated at 37° C. for 1.5 hours. The digestion reactions were then analyzed on 0.8% agarose/TAE gels.

Five positive clones from each truncated ST6 GalNAcI (Colony #1) were inserted into E. coli JM109 cells for expression. To five 1.5 ml autoclaved eppendorf tubes labeled D32, E52, S127, S186 and S201 was added 50 μl of JM109 chemically competent cells, 2.0 μl of mini-prep DNA colony #1 (corresponding to tubes D32, E52, S127, S186 and S201) from construct DH5α/MBP-pCWin2-ST6GalNAcI. The mixtures were incubated on ice for 30 minutes, then heat-shocked for 30 seconds at 42 o C without shaking. Immediately after heat shocking, the tubes were transferred to ice. Room temperature SOC medium (250 μl) was added and the tubes were shaken horizontally at 225 rpm at 37 o C for one hour. A volume of 150 μl of each culture was spread onto LB (50 mg/ml) Kanr agar plates and incubated at 37oC overnight.

DNA from Col. #1 and Col. #2 constructs JM109/MBP-pCWin2-ST6GalNacI (D32, E52, S127, S186 and S201) was then double-digested using restriction enzymes NdeI and XhoI as follows in order to get the MBP-ST6GalNAcI fragment isolated. Digestion conditions are shown in Table 4.

TABLE 4
Digestion conditions for MBP-pCWin2-ST6GalNacI constructs

| | |
|---|---|
| 6.0 μl | DNA from JM109/pCWin2-MBP-GnT1 |
| 2.5 μl | 10X NEB4 Buffer |
| 2.5 μl | 10X BSA |
| 1.5 μl | NdeI enzyme |
| 1.5 μl | XhoI enzyme |
| 11.0 μl | Nuclease free water |

Vials were incubated at 37° C. for 1.5 hours. The digestion reaction then was analyzed on 0.8% agarose/TAE gels.

Mouse GalNAc α2,6-Sialyltransferase (ST6GalNAcI) was expressed from JM109 cells harboring MBP-pCWin2-ST6GalNAcI. 150 ml Martone L-broth containing 10 μg/ml of Kanamycin was innoculated with colony #1 of each N-terminal amino acid truncated construct of JM109/pCWin2-MBP-ST6GalNAcI (D32, E52, S127, S186, and S201). The optical density was monitored at 620 nm until the culture reached an OD of 0.7. Protein expression was induced overnight at 35oC by addition of IPTG (final concentration=500 mM). The next day, the culture was harvested by centrifugation at 4oC, 5000 rpm for 30 minutes. The pellet was resuspended in distilled water. For each gram of pellet, 3.3 ml of water were added. Cells were disrupted using a French press, and the lysed cells were centrifuged at 10000 rpm for 20 minutes. Cell pellets were separated from cell supernatant and an SDS page gel was used to visualize the samples.

SDS-PAGE was conducted using Novex pre-cast 4-20% Tris-Glycine gels in Novex XCELL Electrophoresis System (Invitrogen, Carlsbad, Calif.). Samples were prepared by mixing 50 μl of protein solution with 50 μl of 2× loading buffer and 10 μl of 1M DTT followed by heating at 98 o C for 4 minutes. A volume of 10 μl of each sample was loaded onto the gel and subjected to a constant voltage of 100 V. When the marker dye reached the bottom of the gel, the get was washed with water 3 times for 5 minutes each time. The gel was stained for one hour at room temperature with gentle shaking. The gel was destained with water to obtain a clear background.

TABLE 5
Number of colonies resulting from 100 μl of inoculum for
electroporation of E. coli DH5α.
Table of all transformants

| Constructs | DH5α |
|---|---|
| ST6GalNAcI | No. of colonies |
| D32 | 84 |
| E52 | 372 |
| S127 | 88 |
| S186 | 225 |
| S201 | 232 |

TABLE 6
Number of colonies resulting from 150 μl of inoculum for
electroporation of E. coli JM109 host cells
Table of all transformants

| Constructs | JM109 |
|---|---|
| ST6GalNAcI | No. of colonies |
| D32 | 6 |
| E52 | 21 |
| S127 | 12 |
| S186 | 6 |
| S201 | 21 |

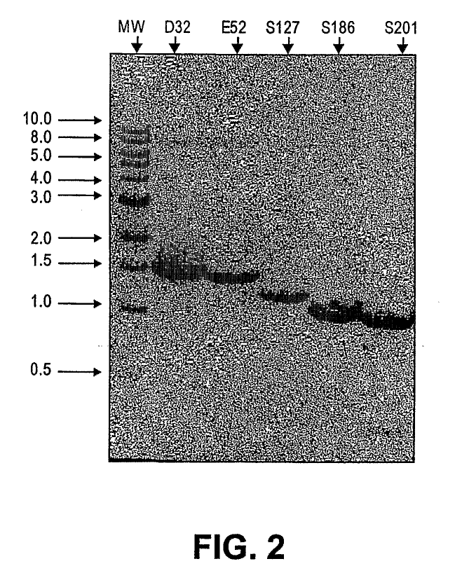

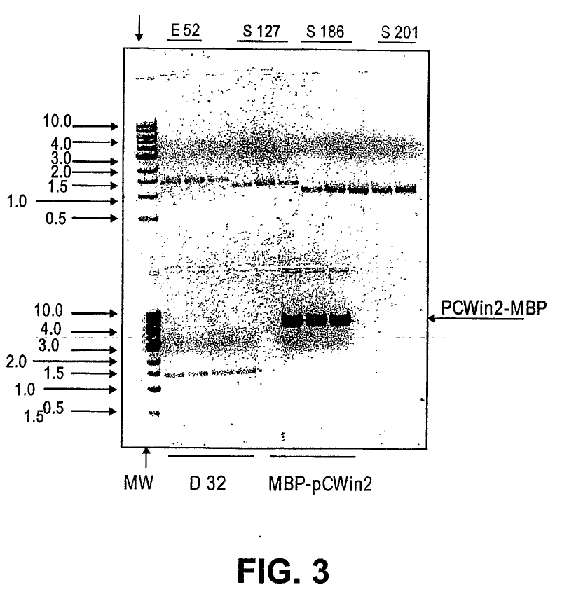

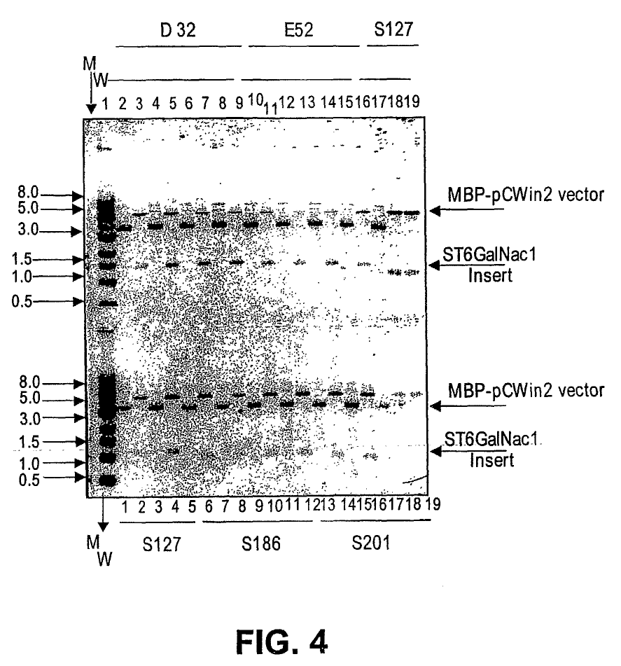

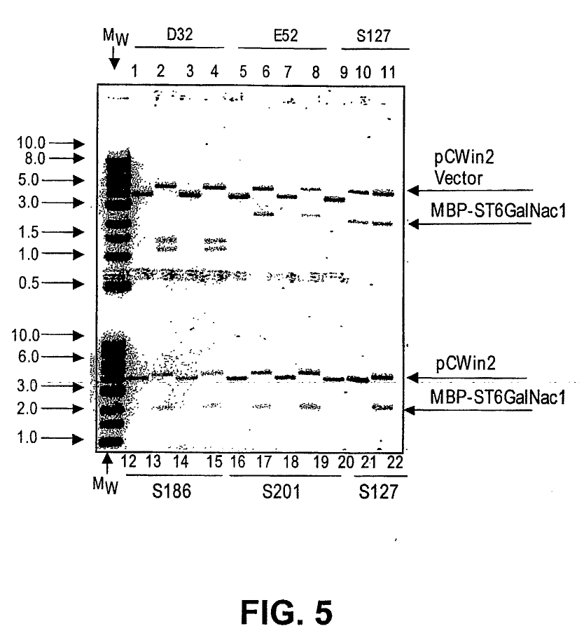

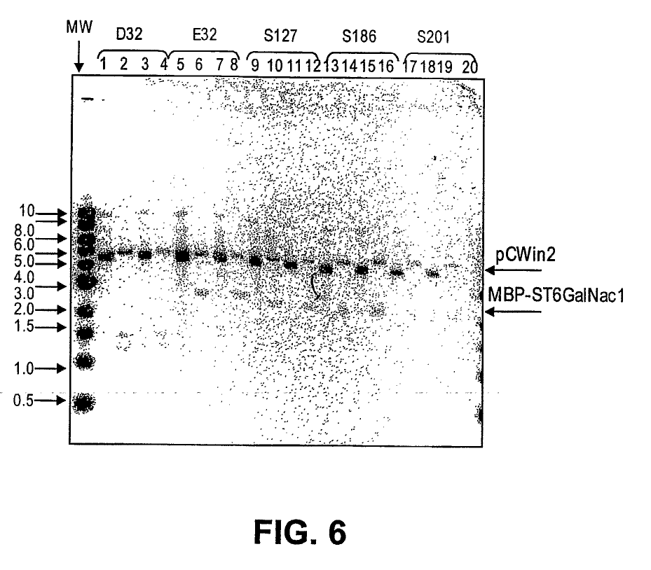

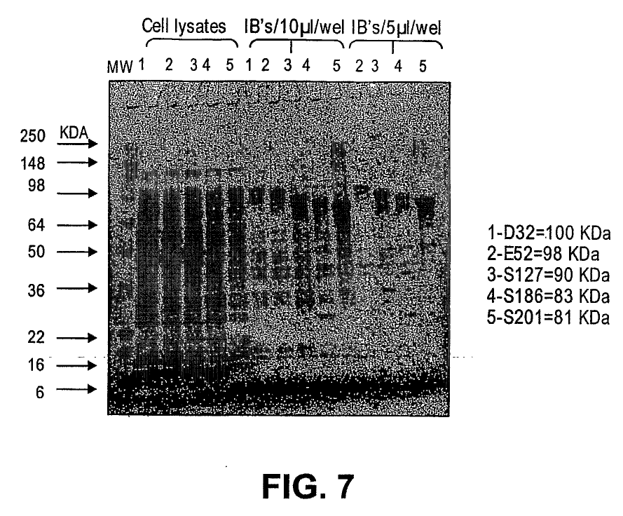

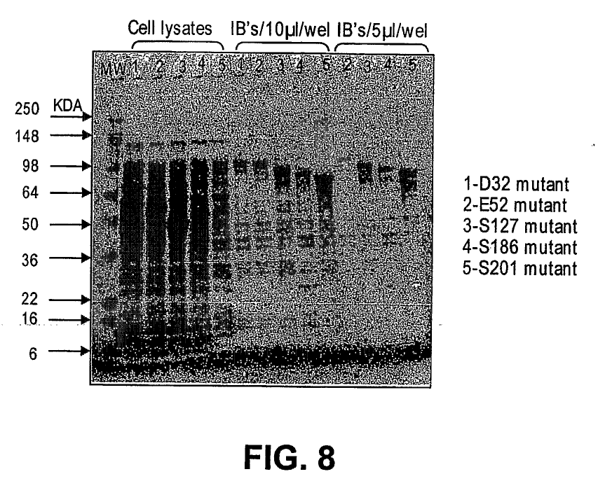

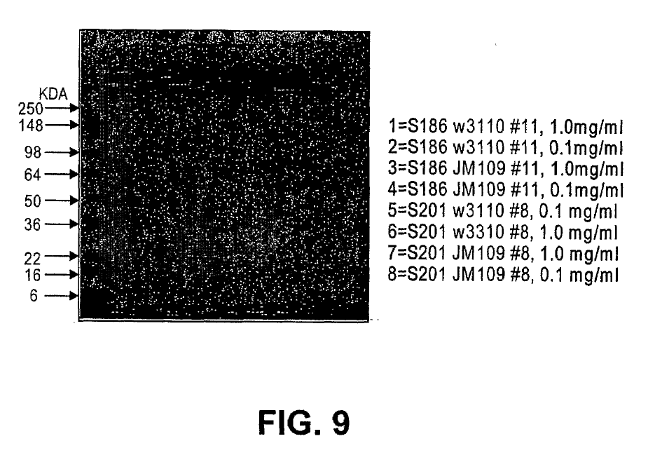

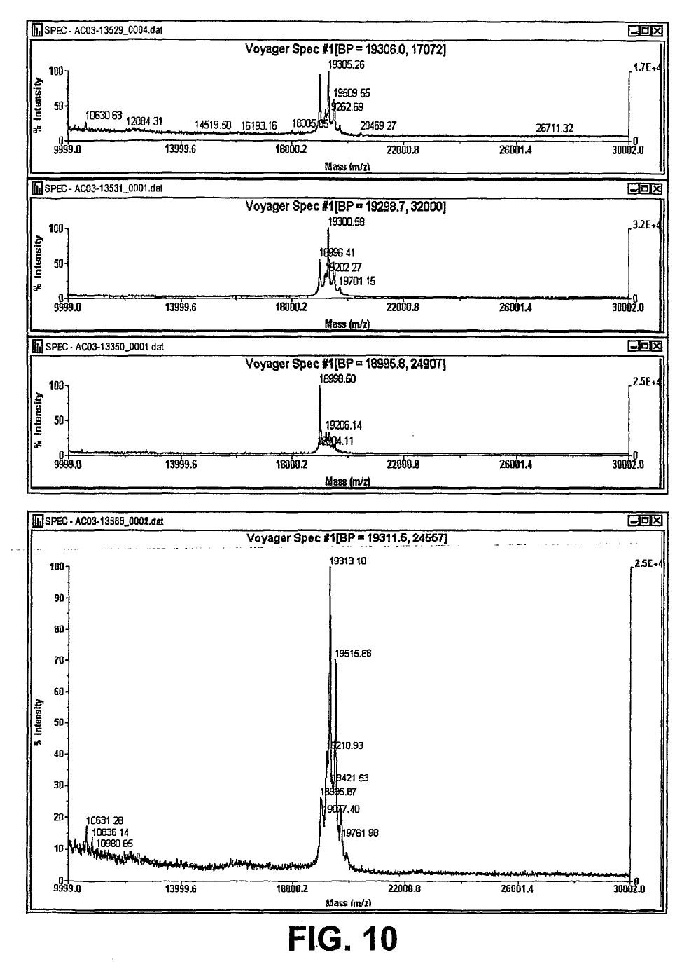

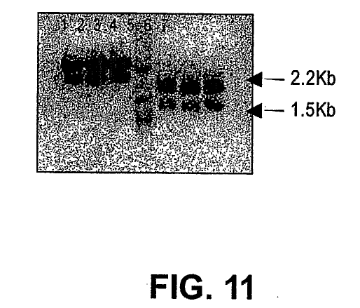

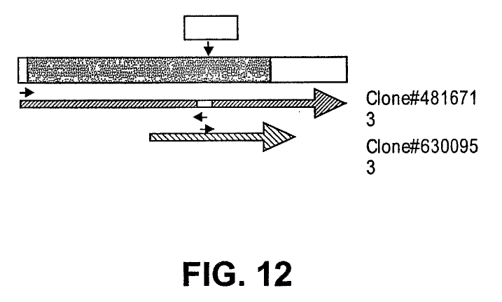

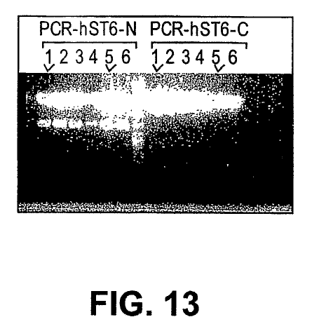

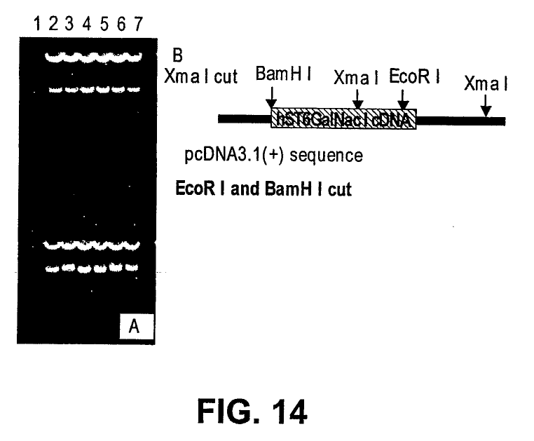

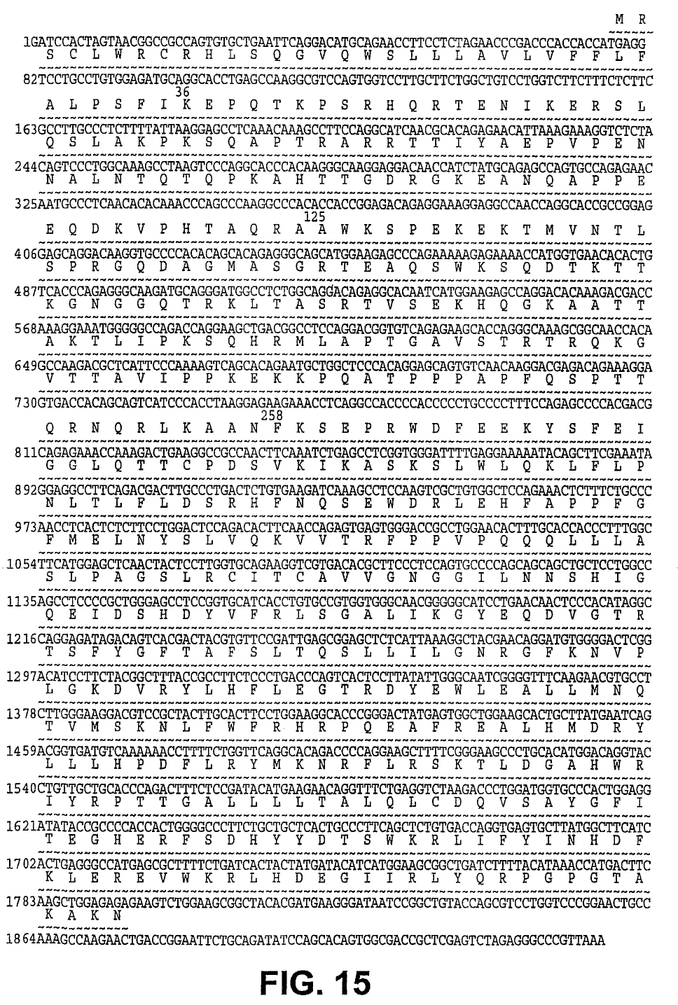

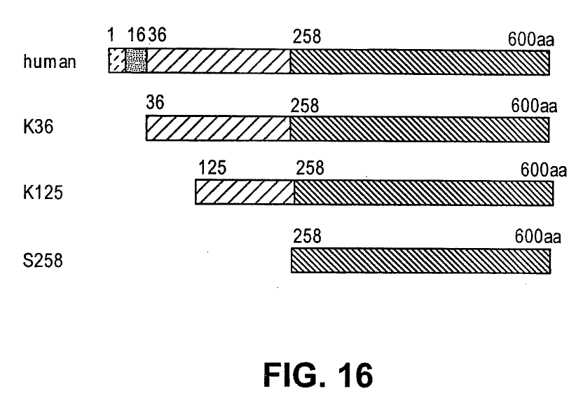

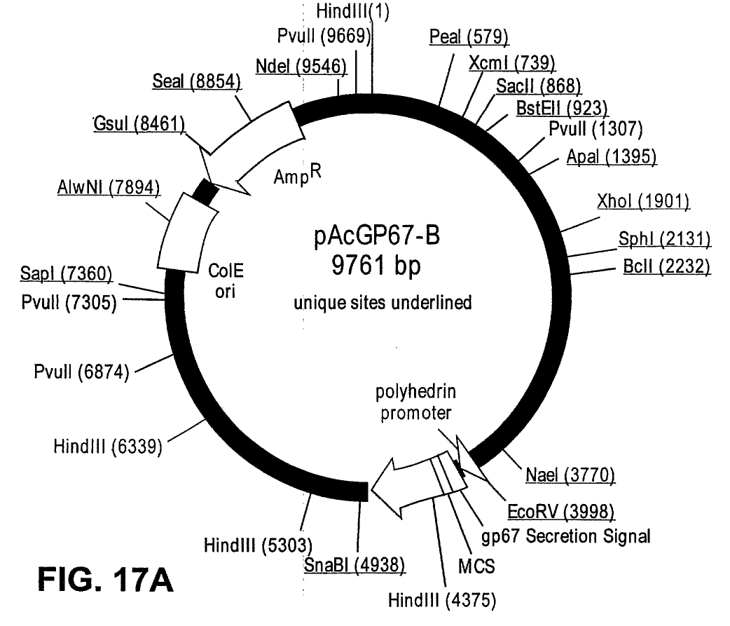

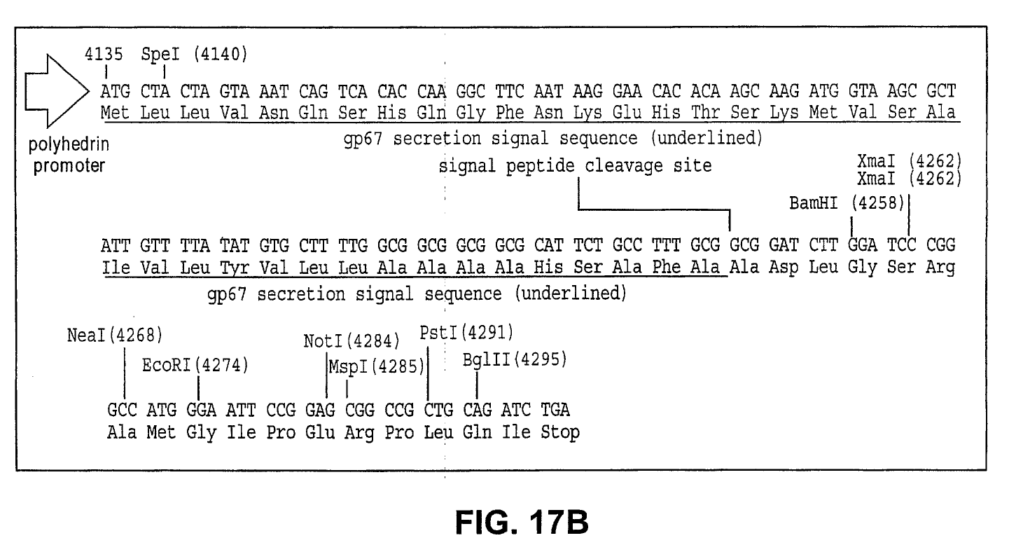

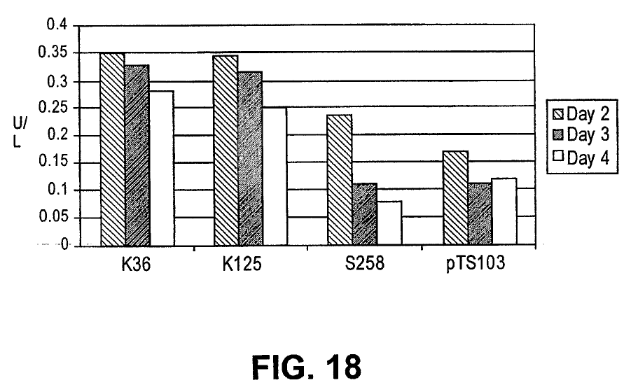

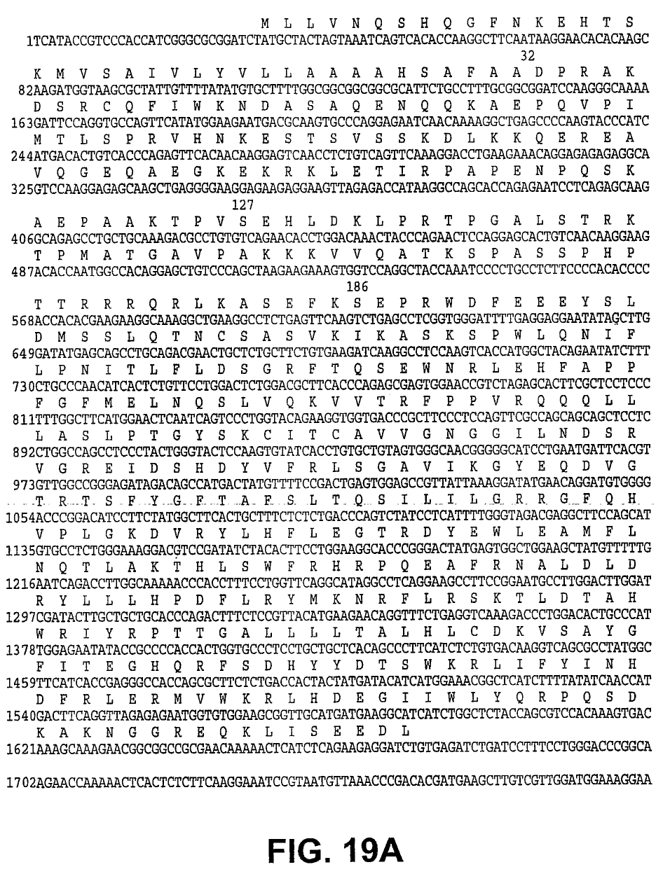

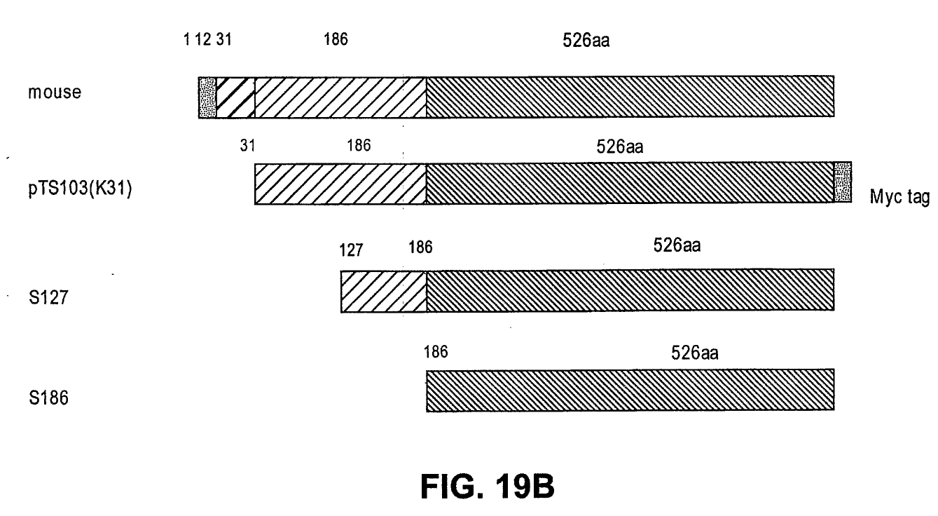

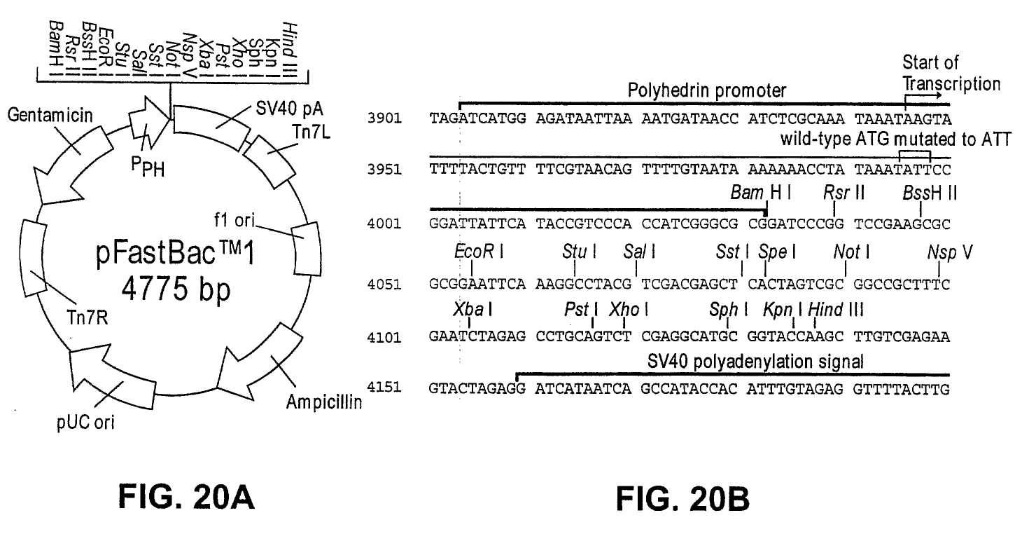

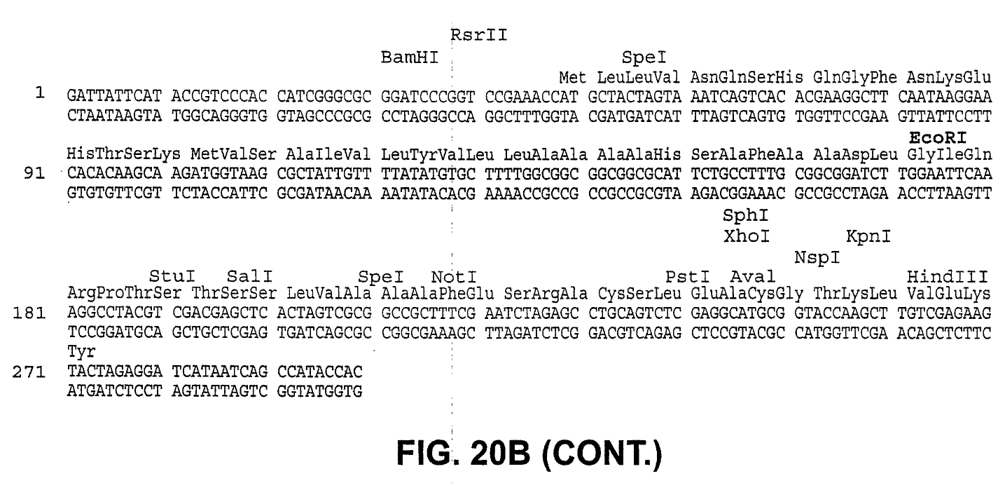

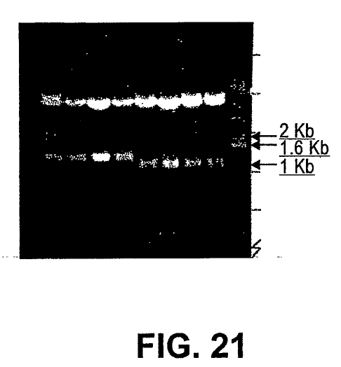

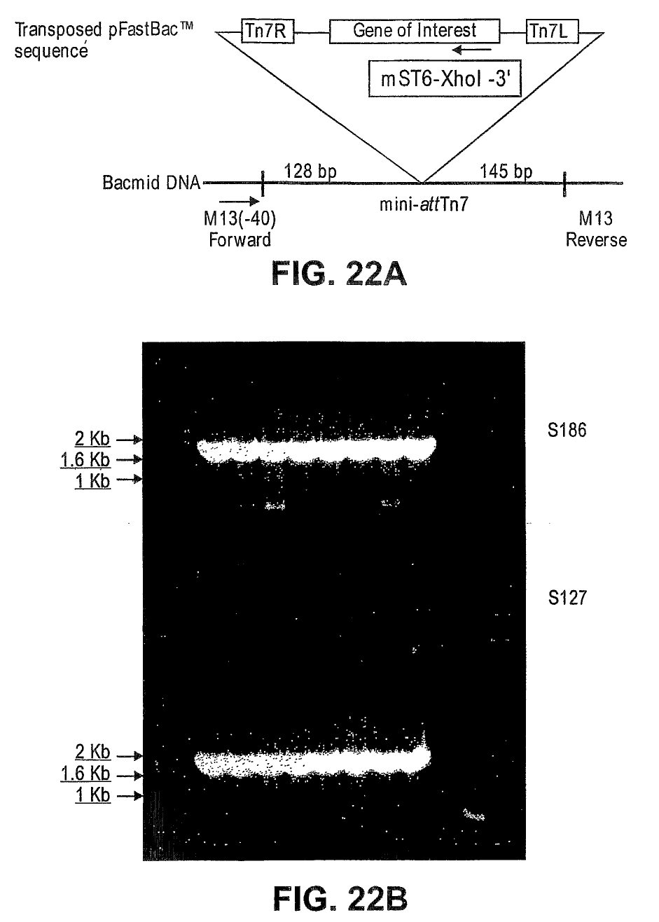

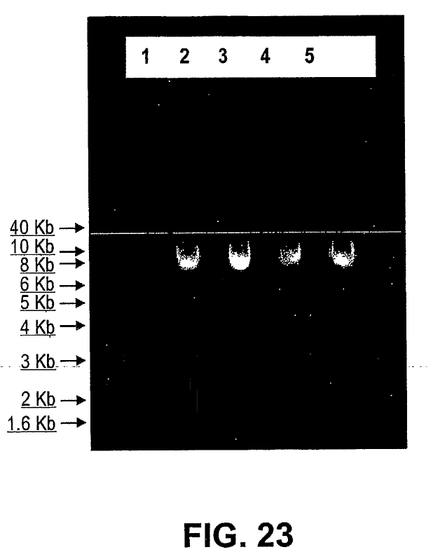

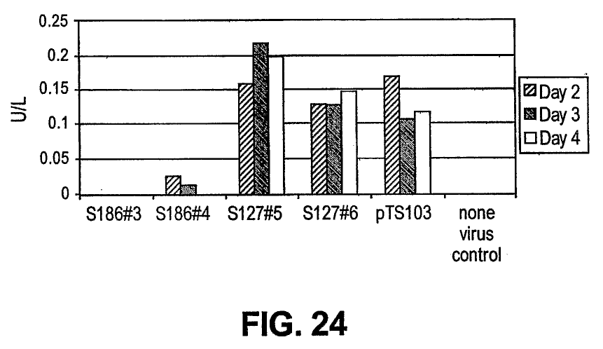

FIG. 2 illustrates the DNA obtained from PCR, after restriction digests using both endonucleases. Expected DNA fragments of 1488 bp, 1428 bp, 1203 bp, 1026 bp, and 981 bp correspond respectively to D32, E52, S127, S186, and S201 of N-terminal amino acid truncated ST6GalNAcI. FIG. 3 illustrates the screening of recombinant colonies DH5α/pCWin2-MBP-ST6GalNAcI, wherein the DNA was digested using HindIII/XhoI restriction enzyme for D32 product and BamHI/XhoI for the constructs E52, S127, S186 and S201 products.

In summary, five mouse N-terminal amino acid truncated GalNAc α2,6-sialyltransferase (ST6GalNAcI) constructs have been successfully cloned and transformed into E. coli DH5α and JM109 host cells, as shown. Construct S201, representing ST6GalNAcI Δ200, was further confirmed by sequence analysis. Fusion proteins have been expressed from E. coli JM109 host cells. The E. coli JM109 transformants have been shown to express the correct size ST6GalNAcI-MBP fusion proteins on SDS page gel.

### Example 2

### Development of Protein Refolding Conditions for E. Coli Expressed MBP-Mouse ST6GalNAcI

E. coli-expressed fusion proteins of Maltose Binding Protein (MBP) and a truncated Mouse GalNac α2,6-Sialyltransferase (ST6GalNAcI) were examined and refolded to produce an active enzyme. For this work, enzyme activity is defined as transfer of sialic acid on to an acceptor protein granulocyte-colony stimulating factor (G-CSF)-O-GalNac by ST6GalNAcI, using a CMP-NAN donor.

Refolding experiments on MBP-ST6GalNAcI were carried out on a 1 ml scale, with five different MBP-ST6GalNAcI DNA constructs and 16 different possible refolding conditions. Refolding was performed using the Hampton Research Foldit kit (Hampton Research, Aliso Viejo, Calif.) and the assays were performed via radioactive detection of CMP [14C] sialic acid addition to a Asialo Bovine Submaxillary Mucin (A-BSM) or Asialo Fetuin (AF), using matrix-assisted laser desorption ionization mass spectrometry (MALDI) analysis utilizing addition of sialic acid to G-CSF-O-GalNAc. The data shows that E. coli-expressed MBP-ST6GalNAcI can be refolded into an active enzyme. Under refold condition 8 found in Hampton Research's Foldit kit (Hampton Research, Aliso Viejo, Calif.), as described herein, active conformations of MBP-ST6GalNAcI construct S201 (serine 201) were obtained. This was validated by a CMP [14C]-sialic acid ST6GalNAcI assay and later confirmed by a GalNAc−O-G-CSF assay.

Glycerol stocks of JM109 pCWin2 MBP-ST6GalNAcI constructs were prepared. Assembly of these constructs is described elsewhere herein. The constructs are comprised of different amino terminal amino acid truncations from the original Mouse ST6GalNAcI protein; including Construct 1—pCWin2 MBP-ST6GalNAcI-D32 Aspartic acid (496aa, 57115.13 MW); Construct 2—pCWin2 MBP-ST6GalNAcI-E52 Glutamic acid (476aa, 54814.77 MW); Construct 3—pCWin2 MBP-ST6GalNAcI-S127 Serine (401aa, 46562.77 MW); Construct 4—pCWin2 MBP-ST6GalNAcI-S186 Serine (342aa, 40160.65 MW); and Construct 5—pCWin2 MBP-ST6GalNAcI-S201 Serine (327aa, 38245.82 MW).

Constructs were grown in 150 ml Martone L-Broth cultures containing 10 μg/ml Kanamycin sulfate. Each culture was inoculated with one isolated colony corresponding to constructs #1 through #5. The 150 ml cultures were incubated overnight at 37° C., shaking at 250 rpm. Starter cultures of 5 ml Martone L-Broth containing 10 μg/ml Kanamycin sulfate were inoculated with one isolated colony of construct S186 and S201. This procedure was performed for a total of four starter cultures. Starter cultures were incubated overnight at 37° C., shaking at 250 rpm.

Lastly, two 1 L Martone L-Broth cultures containing 10 μg/ml Kanamycin sulfate were prepared. Each of these cultures was inoculated with 5 ml of over night starter culture of constructs S186 or S201. These 1 L cultures were incubated at 37° C., with shaking at 250 rpm, until the OD620 measured in a range of 0.6 to 1.0. Upon reaching this point, IPTG was added to each of the two 1 L cultures to a final concentration of 0.5 mM. Cultures were then allowed to continue incubating overnight at 37° C., with shaking at 250 rpm. In addition, two fermenter vessels containing 1½ liter of Martone L-Broth with 10 μg/ml Kanamycin was inoculated with 5.0 ml of starter culture with following unit specifications: temperature 37.0, pH=7.0.

Cultures (150 ml) of JM109 pCWin2 MBP-ST6GalNAcI constructs 1 through 5 were transferred to 250 ml centrifuge bottles. Cultures were then centrifuged at 5000 rpm for 30 minutes at 4° C. Supernatants were removed and the pellets were weighed. The pellets from each sample were then washed to isolate the inclusion bodies (IBs). The pellet of each construct was first resuspended in 6.0 ml of 20 mM Tris-HCl, 5 mM EDTA, pH-9 and then lysed by adding 25 μl of 20 mg/ml lysozyme and 10 μl of 1 mg/ml DNase1. The reaction tubes then were incubated at 37oC for one hour.

The lysates for each construct were then centrifuged at 10,000 rpm at, 4oC for 15 minutes. The supernatants were removed and the pellets were resuspended in 6.0 ml of 20 mM TrisHcl, 5 mM EDTA, pH=6.5. The supernatants were then removed and the pellets were resuspended a second time in 6.0 ml of 20 mM Tris-Hcl, 5 mM EDTA pH=6.5. The suspensions were then centrifuged at 5000 rpm, 25° C. for 5 minutes. The supernatants were removed and a third wash was performed by resuspending the pellets in 6.0 ml of 20 mM Tris-HCl, pH=6.5, 5 mM EDTA. The suspensions were then centrifuged at 5000 rpm, 25° C. for 5 minutes. The supernatants from each sample were removed and the pellets were weighed and stored at −20oC. SDS-PAGE was conducted using both the lysates and the pellets by adding 50 μl of the sample and 50 μl of 2× loading buffer and 10 μl of 1.0 M DTT heating at 98oC for 6 minutes. Expression of the protein was observed in the gel. The pellets were then weighed and resuspended with 1.0 ml of 20 mM Tris-HCl pH=6.5, 5 mM EDTA. 1 ml aliquots were made for each of the five constructs and used for analysis. These aliquots represent the triple washed inclusion bodies (TWIsB).

Cultures from JM109/pCWin2-MBP-ST6GalNAcI constructs S186 and S201 in shaker flasks and fermenters were transferred to 1 L centrifuge bottles. Cultures were then centrifuged at 5000 rpm for 30 minutes at 4oC. Supernatants were removed and the pellets were weighed. The pellets from each sample were then washed to isolate the inclusion bodies (IB's). The pellets of S186 and S201 were first resuspended in 35 ml of 20 mM Tris-HCl, PH=8-0, 5 mM EDTA and then lysed by two passages through the French press at 12,000 psi.

The lysates for each construct were then centrifuged at 5000 rpm, 25oC for 5 minutes in 50 ml disposable tubes. The supernatants were removed and the pellets were resuspended in 35 ml of 20 mM Tris HCl, pH=6.5, 5 mM EDTA. The suspensions were then centrifuged and the samples were resuspended a second time in 35 ml of 20 mM Tris-HCl, pH-6.5, 1% Triton X-100. The suspensions were again centrifuged at 5000, 25oC for 5 minutes. The supernatants were removed and a third wash was performed by resuspending the pellets in 35 ml of 20 mM tris-HCl pH=6.5, 5 mM EDTA. The suspensions were then centrifuged at 5000 rpm, 25° C. for 5 minutes. The supernatants from each sample were removed and the pellets were weighed and stored at −20oC.

Solubilization buffer was prepared with the following concentrations of materials: 6M Guanidine HCl, 5 mM EDTA, 50 mM Tris-HCl, pH=6.5 and 10 mM DTT. 1 ml of this solution was added to 20 mg TWIBs to yield a 20 mg/ml solution. The solution was incubated overnight on the bench top to solubilize IBs. This procedure was performed on a TWIB aliquot of each MBP-ST6GalNAcI construct to provide protein for refolding experiments. Protein samples from each construct were diluted by combining 950 μl of IB solubilized butter with 50 μl of protein sample. Samples were then analyzed by UV Spectrophotometer and the protein concentration and percent protein solubilized conversion was calculated from those values and the molar extinction coefficient: Construct D32-1.24 mg/ml per 1 A280 unit, Construct E52-1.29 mg/ml per 1 A280 unit, Construct S127-1.52 mg/ml per 1 A280 unit, Construct S186-1.77 mg/ml per 1 A280 unit, construct S201-1.38 mg/ml per 1 A280 unit.

Protein refold samples were purified using Harvard Bioscience G-50 Macro Spin Columns (Holliston, Mass.). Caps were removed from the G-50 columns and these were placed into 2 ml microcentrifuge tubes. 500 μl of water was added to each column and they were then allowed to incubate for 15 minutes to hydrate. The columns were then centrifuged at ˜2000×g for 4 minutes after which they were transferred to new 2 ml centrifuge tubes. 1501 of each refold solution was applied to one of the columns. Columns were then centrifuged at 2000×g for 2 minutes. Resulting permeates represented the purified refold samples. An SDS gel was used to visualize the purified protein

To screen refolding conditions that may result in an active form of E coli expressed MBP-ST6GalNAcI, a Hampton Foldit Screening kit (Hampton Research, Aliso Viejo, Calif.) was utilized. The composition of each of the refolding buffers is set forth elsewhere herein. For a given refolding condition, 950 μl of refolding buffer was combined with 50 μl of solubilized protein (for high protein concentration conditions) or 950 μl of refolding buffer was combined with 50 μl of 1:10 dilution of the high protein concentration of solubilized protein (for low protein concentration conditions). Refolding reactions were placed on a rotator in a cold room (4° C.), rotating overnight.

A radiolabeled [14C] CMP-sialic acid assay was performed to determine the activity of the E. coli expressed refolded MBP-ST6GalNAcI by monitoring the addition of radiolabel to Asialo Fetuin (AF) or A-BSM (Asialo Bovine Submaxillary glands Mucin) acceptor. 50 mg of AF was dissolved in 11.0 ml of water to have an initial concentration of 50 mg/ml. A-BSM was prepared by release of sialic acid by means of hydrolysis from BSM (mucin, type 1-S). The initial screen was performed on refolded protein samples obtained in 150 ml cultures. Subsequent refold samples were also refolded and purified from one liter cultures for construct S201 and S186. The assay included protein samples, ST6GalNAcI from baculovirus as a positive control, a negative control sample with all the components except acceptor and a maximum input sample which contained all components except enzyme. A total of 20 samples were tested. The 14C ST6GalNAcI assay reaction mixture included 50 mg/ml A-BSM or AF at 0.25 mg, in 50 mM MES pH 6.0, 100 mM NaCl 40 nCi [14C]-CMP-sialic acid, 0.2 mM cold CMP sialic acid, with 10 μl enzyme solution.

For each of the refolding samples, 40 μl of the reaction mixture were combined with 10 μl of the refolding samples. For the negative control 10 μl H2O was combined with 40 μl of the reaction mixture. Positive control was treated the same as samples that is addition of 10 μl of ST6GalNAcI baculovirus enzyme supernatant was added to 40 μl reaction mixture. For the maximum input sample 40 μl of the reaction mixture was combined with 10 μl of dH2O. Reactions were incubated at 37° C. for 60 minutes. Reactions were stopped by addition of 100 μl of mixture of 5% phosphotungstic acid/15% TCA. The reaction mixture was microfuged at 10000 rpm for two minutes. Supernatant was removed by pipetting and the sediments were washed with 500 μl of 5% TCA and vortexed. The mixture was microfuged at 10,000 rpm for two minutes and the supernatant was removed by pipetting. The pellets were resuspended in 100 μl of 10N NaOH. One-ml of H2O was added to each reaction; samples were vortexed briefly and then loaded into scintillation vials. Five-ml of scintillation cocktail was added to each of the samples and controls Samples were shaken briefly and loaded on the scintillation counter and radioactivity measured.

A G-CSF assay was performed to determine whether E. coli-expressed refolded MBP-ST6GalNAcI, in the presence of CMP-NAN, could transfer sialic acid to a GalNAc−O-G-CSF acceptor. ST6GalNAcI construct S 186 (refold buffers #8 and #11) and construct S201 (refold buffer # 8) were assayed for transferase activity. Additionally, as a positive control, ST6GalNAcI from Baculovirus was assayed. The assay included GalNAc−O-GCSF (100 μg), CMP-NAN (0.750 mg), MES buffer, pH 6.0, and MnCl2 (100 mM). Table 7 illustrates the silayltransferase reaction as cataylzed by the enzyme obtained by refold condition #8.

TABLE 7
Sialyltransferase activity of enzyme obtain under refolding condition #8.
Transfer of Sialic acid by Bacterial ST6GalNAcI refold #8 to
GalNac-G-CSF.

| Reaction mixture | A | B |
|---|---|---|
| 1-GalNAc G-CSF 1 μg/μl | 50 μl | 50 μl |
| 2-MnCl2 100 mM | 5.0 μl | 5.0 μl |
| 3-CMP-NAN | 5.0 μl | 5.0 μl |
| ST6GalNAc I | 50 μl | 100 μl |

GalNAc-G-CSF dissolved in 25 mM of MES Buffer + 0.05% of Na azide
pH = 6.0.
CMP-NAN 0.75 g in 100 μl of MES Buffer.
ST6GalNAc I Refold #8.
Incubate reaction tubes at 32° C. with gentle shaking.
Take out 5.0 μl each time and submit for MALDI-TOF analysis.

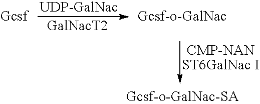

At different time intervals (2, 24, 48, and 120 hrs), aliquots of samples were subjected to MALDI-time of flight (TOF) analysis. Results clearly indicate transfer of sialic acid to GalNac-O-G-CSF.

Pellet weights and inclusion body weights were determined for each of the five 150 ml JM109 pCWin MBP-ST6GalNAcI, representing cultures 1 through 5:

TABLE 8
Pellet and Inclusion Body Weights from 150 ml JM109 pCWin2
MBP-ST6GalNAcl Cultures

| JM109 pCWin2 MBP- | | Inclusion |
|---|---|---|
| ST6GalNAcI | Cell Pellet Weight | Body Weight |
| Constructs | (g) | (g) |
| D32 | 0.65 | 0.30 |
| E52 | 0.98 | 0.73 |
| S127 | 0.56 | 0.57 |
| S186 | 1.2 | 0.93 |
| S201 | 1.1 | 0.83 |

Pellet weights and inclusion body weight were determined for cultures in 1 L shaker flasks and 1.5 L fermenters including JM109 pCWin MBP-ST6GalNAcI constructs S186 and S201 cultures. Protein samples were diluted and concentration was measured at OD₂₈₀. Protein concentration and percent of solubilized protein conversions were calculated for all five truncated ST6GalNAcI clones, as set forth in Table 9.

TABLE 9
Pellet and Inclusion Body Weights from 1 L Shaker flasks and 1½ L
Fermenters JM109 pCWin2 MBP-ST6GalNAcI Cultures

| JM109 pCWin2 MBP- | | Inclusion |
|---|---|---|
| ST6GalNAcI | Cell Pellet Weight | Body Weight |
| Constructs | (g) | (g) |
| S186 Shaker flask | 10.2 | 2.30 |
| S201 Shaker flask | 8.22 | 2.94 |
| S186 Fermenter | 14.33 | 1.47 |
| S201 Fermenter | 12.48 | 2.67 |

Protein Concentration and % conversion of 150 ml. JM109 pCWin2
MBP-ST6GalNAcI cultures after Solubilization.

| JM109 | | | | |
|---|---|---|---|---|
| pCWin2 | | Protein | | |
| MBP- | | Con- | Protein | Protein |
| ST6GalNAcI | A₂₈₀ After | centration | % of | Concentration |
| Constructs | Solubilization | (mg/ml) | Conversion | (mg/ml) |
| D32 | 0.113 | 4.56 | 3.9 | 1.0 and 0.1 |
| E52 | 0.129 | 5.00 | 2.5 | 1.0 and 0.1 |
| S127 | 0.153 | 5.03 | 5.5 | 1.0 and 0.1 |
| S186 | 0.201 | 5.68 | 2.3 | 1.0 and 0.1 |
| S201 | 0.274 | 9.93 | 12.4 | 1.0 and 0.1 |

TABLE 10
Protein refolding conditions used with the Hampton Research Foldit kit

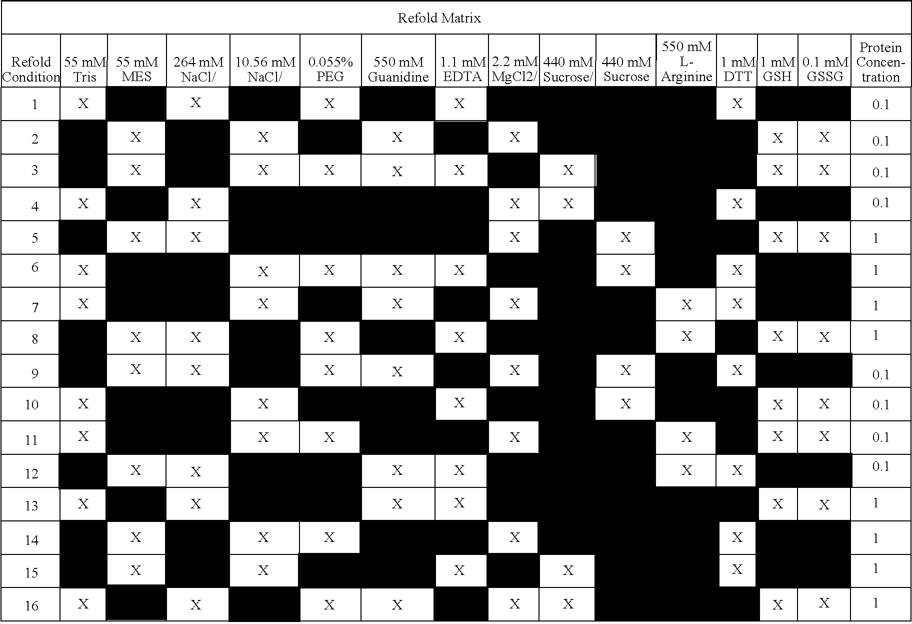

TABLE 11
Results from initial refold buffer screen.
In this assay, all five constructs were tested under all 16 refold conditions from the
Hampton Foldit kit (Hampton Research, Aliso Viejo, CA). These refolds were purified by G-
50 gel filtration and then tested for activity by the radioactive assay as described above.

| Refold | | | | | | | |
|---|---|---|---|---|---|---|---|
| condition | | | | | | | |
| 1 | 2 | 3 | 4 | 5 | 6 | 7 | 8 |

Raw CPM

| | | | | | | | | |
|---|---|---|---|---|---|---|---|---|
| D32 | 78 | 38 | 131 | 54 | 53 | 44 | 47 | 160 |
| E52 | 142 | 165 | 346 | 155 | 136 | 178 | 133 | 152 |
| S127 | 126 | 345 | 381 | 267 | 238 | 186 | 247 | 166 |
| S186 | 341 | 373 | 779 | 335 | 289 | 180 | 337 | 386 |
| S201 | 3387 | 2892 | 3496 | 1566 | 2077 | 1580 | 2851 | 5186 |
| Sf9 + Control | 1942 | | | | | | | |
| Negative | 93 | | | | | | | |
| Control | | | | | | | | |

Corrected CPM

| | | | | | | | | |
|---|---|---|---|---|---|---|---|---|
| D32 | 42 | 2 | 95 | 18 | 15 | 8 | 11 | 124 |
| E52 | 38 | 61 | 242 | 51 | 32 | 74 | 29 | 48 |
| S122 | −64 | 155 | 191 | 77 | 48 | −4 | 57 | −24 |
| S186 | 300 | 332 | 738 | 294 | 248 | 139 | 296 | 345 |
| S201 | 1382 | 887 | 1491 | −439 | 72 | −425 | 846 | 3181 |

% CPM

| | | | | | | | | |
|---|---|---|---|---|---|---|---|---|
| D32 | 0.08 | 0.00 | 0.19 | 0.04 | 0.03 | 0.02 | 0.02 | 0.25 |
| E52 | 0.05 | 0.08 | 0.32 | 0.07 | 0.04 | 1.00 | 0.04 | 0.06 |
| S127 | −0.08 | 0.18 | 0.23 | 0.09 | 0.06 | 0.00 | 0.07 | −0.03 |
| S186 | 0.37 | 0.41 | 0.90 | 0.36 | 0.30 | 0.17 | 0.36 | 0.42 |
| S201 | 1.64 | 1.06 | 1.78 | −0.52 | 0.09 | 0.051 | 1.01 | 3.79 |

| Refold | | | | | | | |
|---|---|---|---|---|---|---|---|
| condition | | | | | | | |
| 9 | 10 | 11 | 12 | 13 | 14 | 15 | 16 |

Raw CPM

| | | | | | | | | |
|---|---|---|---|---|---|---|---|---|
| D32 | 67 | 46 | 160 | 35 | 150 | 79 | 39 | 32 |
| E52 | 286 | 158 | 298 | 226 | 178 | 150 | 367 | 205 |
| S127 | 196 | 125 | 268 | 274 | 210 | 149 | 159 | 216 |
| S186 | 330 | 302 | 1795 | 447 | 289 | 465 | 2476 | 358 |
| S201 | 7665 | 1099 | 3158 | 2932 | 2585 | 871 | 2559 | 2343 |
| Sf9 + Control | | | | | | | | |
| Negative | | | | | | | | |
| Control | | | | | | | | |

Corrected CPM

| | | | | | | | | |
|---|---|---|---|---|---|---|---|---|
| D32 | 31 | 10 | 124 | −1 | 114 | −7 | 3 | −4 |
| E52 | 182 | 54 | 194 | 122 | 74 | 46 | 263 | 101 |
| S122 | 6 | −65 | 78 | 84 | 20 | −41 | −31 | 26 |
| S186 | 289 | 261 | 1754 | 406 | 248 | 424 | 2435 | 317 |
| S201 | 5660 | −906 | 1153 | 927 | 580 | 1134 | 554 | 338 |

% CPM

| | | | | | | | | |
|---|---|---|---|---|---|---|---|---|
| D32 | 0.06 | 0.02 | 0.25 | 0.00 | 0.23 | −0.01 | 0.00 | 0.00 |
| E52 | 0.24 | 0.07 | 0.26 | 0.16 | 1.00 | 0.06 | 0.35 | 0.13 |
| S127 | 0.00 | −0.08 | 0.09 | 1.00 | 0.02 | −0.05 | −0.04 | 0.03 |
| S186 | 0.35 | 0.32 | 2.15 | 0.50 | 0.3 | 0.52 | 2.9 | 0.39 |
| S201 | 6.74 | −1.08 | 1.37 | 1.1 | 0.69 | −1.35 | 0.66 | 0.40 |

Results from this radioactive assay indicated that refold conditions 8 and 9 worked best for construct S201. Two conditions—8 and 11—for construct S186, condition 12 for construct S127 and condition 6 for construct E52 provided the highest CPM count.

TABLE 12
Results from S201 Construct Refold # 8 and # 9.
In this assay, construct S201 was re-tested under refold conditions 8 and 9 with 1.0
and 0.1 mg/ml concentration with and without DTT from the Hampton Foldit kit (Hampton
Research, Aliso Viejo, CA). The refolded proteins were purified by G-50 gel filtration and
then tested for activity by the radioactive assay. Results indicate that Refold # 8 holds higher
CPM counts than refold # 9.

| | | | | | | ¹⁴C Activity | | | | | |
|---|---|---|---|---|---|---|---|---|---|---|---|
| | | Foldit Screen | | ST6GalNAcI/S201 | | Assay | | | Raw | | |
| Beffer # | pH | Tris | MES | Detergent | Polar/Non | DTT | GSH/GSSG | mg/ml | CPM | Corr.CPM | % CPM |
| 8-1 | 6.5 | − | + | + | Arginine | − | +/+ | 1.0 | 4570 | 2678 | 3.19 |
| 8-2 | 6.5 | − | + | + | Arginine | − | +/+ | 0.1 | 3296 | 1404 | 1.67 |
| 9-1 | 6.5 | − | + | + | Sucrose | − | +/+ | 0.1 | 2192 | 561 | 0.67 |
| 9-2 | 6.5 | − | + | + | Sucrose | − | +/+ | 1.0 | 1472 | −159 | −0.19 |
| NA + 8-1 | | | | | | | | | 1892 | | |
| NA + 9-2 | | | | | | | | | 1631 | | |
| A-Enz | | | | | | | | | 2560 | | |
| NA/NE | | | | | | | | | 4397 | | |
| Cont. = 2 μl | | | | | | | | | 84000 | | |

| | | | | | |
|---|---|---|---|---|---|
| Blank | NA = No | NE = No | A-E = Acceptor- | AM644- | 27 |
| | Acceptor | Enzyme | Enzyme | pg46 | |

TABLE 13
Results of the repeat of Refold # 11 of S186 and Refold # 8 of S201.
These proteins were used to analyze transfer of Sialic acid to G-csf-O-
GalNac(AM644-pg150-156).

| | | | | | | ¹⁴C Activity | | | | | |
|---|---|---|---|---|---|---|---|---|---|---|---|
| | | Foldit Screen | | ST6GalNAcI/S186 | | Assay | | | Raw | Corr. | |
| Beffer # | pH | Tris | MES | Detergent | Polar/Non | DTT | GSH/GSSG | mg/ml | CPM | CPM | % CPM |
| 11-1 | 8.2 | + | − | − | Arginine | − | +/+ | 1.0 | 520 | 484 | 0.6 |
| 11-2 | 8.2 | + | − | − | Arginine | − | +/+ | 0.1 | 695 | 581 | 0.7 |
| 8-1 | 6.5 | − | + | + | Arginine | − | +/+ | 0.1 | 784 | 748 | 0.8 |
| 8-2 | 6.5 | − | + | + | Arginine | − | +/+ | 1.0 | 206 | 170 | 0.2 |
| BV + Cont | | | | | | | | | 2392 | 2356 | 2.7 |
| −Acp/ | | | | | | | | | 36 | | |
| +Enz | | | | | | | | | | | |
| +Acp/−Enz | | | | | | | | | 54 | | |
| Cont. = 2 μl | | | | | | | | | 88491 | | |

| | | | | | |
|---|---|---|---|---|---|
| Blank | NA = No | NE = No | A-E = Acceptor- | AM644-pg46 | 29 |
| | Acceptor | Enzyme | Enzyme | | |

From results obtained in the screening process, it was determined that refold conditions 8 and 9 for S201 and conditions 8 and 11 for S186 yielded the most promising results. To achieve reproducibility, additional refolding reactions were performed under the same conditions using G-50 gel filtration for refolds 8 and 9 for S201 and 8 and 11 for S186. From these experiments, refold 8 yielded higher counts and was found to be reproducible while conditions 9 and 11 did not.

A granulocyte-colony stimulating factor (G-CSF) assay was performed with refolded proteins of constructs S186 and S201 in refold buffer 8 The G-CSF reaction was allowed to incubate at 32° C. for 5 days. The reaction was analyzed at 1, 2 and 16 hours and at 1, 2 and 5 days time points. The parental peak for GalNAc−O-G-CSF is expected at MW ˜19006. A successful reaction is indicated by addition of ˜309 and 509 molecular weight to that peak. From the 5 days data for refolds S201 a developing peak was seen at ˜19313 (GalNAc+SA) and 19515 (2GalNAc+SA), a difference of approximately 307 and 509. This data again illustrated that sialic acid was added to GalNAc−O-G-CSF by the refolded truncated mouse ST6GalNAcI proteins and confirmed what was reported by the radioactive assay.

These data support the conclusion that refolded E. coli-expressed MBP-ST6GalNAc 1 is an active sialyltransferase enzyme, and that under refold condition 8 found in Hampton Research's Foldit kit (Hampton Research, Aliso Viejo, Calif.), active conformations of MBP-ST6GalNAcI construct S201 (Δ200) are achievable. The generation of a functional refolded protein was demonstrated in the [14C] radioactive and GalNAC−O-G-CSF assays.

### Example 3

### Cloning and Expression of Human and Mouse GalNAc α-2,6-Sialyltransferases (ST6GalNAcI) in a Baculovirus Expression System

The expression of both human and mouse GalNAc α2,6-sialyltransferases (ST6GalNAcI) was demonstrated in Sf9 (insect) cells. To examine the expression of human GalNAc α-2,6-sialyltransferase (hST6GalNAcI) in Sf9 (insect) cells, the long form of full-length human cDNA was constructed by PCR cloning of two EST clones into pcDNA3.1(+)(GenBank accession number is Y11339; there is a shorter form of cDNA in the NCBI data base). Three truncated forms of hST6GalNAcI, K36, K125 and S258 (corresponding to Δ35, Δ124 and Δ257) were cloned into the baculovirus vector pAcgp67B based on this hST6GalNAcI clone. All three truncations can be expressed in Sf9 cells and K36 showed the highest activity. A mouse ST6GalNAcI in a baculovirus expression vector in pAcgp67A called pTS103 (K31 truncation, corresponding to Δ30) was also obtained. Two additional truncations, S127 and S186 (corresponding to Δ126 and Δ185) were made and expressed in the baculovirus vector pFastBac-1-gp (Invitrogen, Carlsbad, Calif.). Expression studies on these three truncations showed that S127 has the highest expression level.

Described herein are the processes of cloning and expression of both human and mouse GalNAc α-2,6-Sialyltransferases (ST6GalNAcI) in Sf9 (insect) cells, including the source of cDNAs, detail description of steps in the assembly of final expression plasmid, the expression and the enzymatic activities of the secreted proteins.

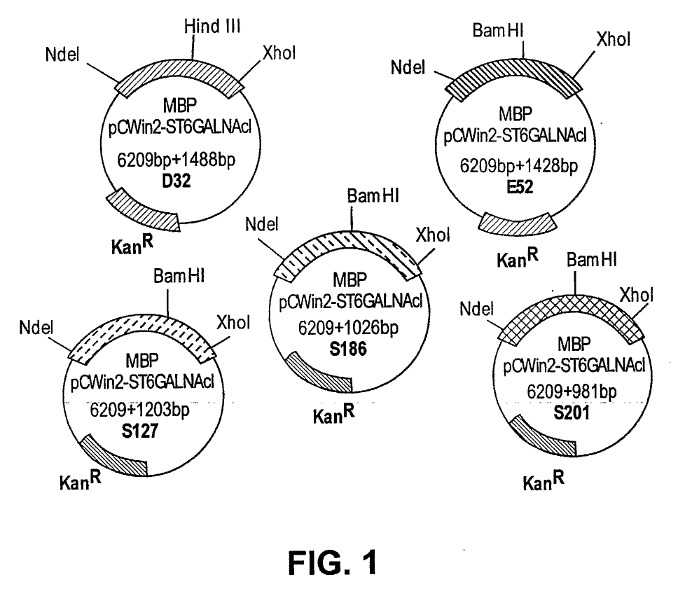

Two human EST clones containing two fragments of human ST6GalNAcI (the clone IDs are 4816713/Cat#97002RG and 6300955/Cat#97002RG) were obtained from invitrogen. These clones were obtained as bacterial glycerol stocks in tubes on dry ice. The bacterial stocks were streaked on a LB agar plate containing ampicillin for clone #4816713 and on a LB agar plate containing chloramphenicol for clone# 6300955. The plates were incubated at 37° C. overnight. Three individual colonies were picked and inoculated into 5 ml LB culture. DNA plasmid was isolated using QIAprep Spin Miniprep Kit (Qiagen, Valencia, Calif.). Enzymatic digestions showed that clone #4816713 has an insert of about 2-2 Kb released by EcoRI and clone# 6300955 has an insert of about 1.5 Kb released by EcoR I and Xho I (FIG. 1). Both clones released the expected sizes of inserts.

By comparing the sequences to the published human ST6GalNAcI (long form, accession# Y11339), it is clear that clone #4816713 covers the entire sequence except a fragment from nucleotide #f 1375 to 1480. Clone# 6300955 covers sequences from nucleotide #1070 to the C-terminus. Therefore, two sets of PCR primers were designed for cloning the full length human ST6GalNAcI cDNA. The first set of primers is: hST6GN1-F1, caGGATCCacatgcagaaccttcc (SEQ ID NO:49) and hST60N1-R2, gtcccgggtcgccttccaggaagtgeaagtagcggacgtccttcccaagaggcacg (SEQ ID NO:50). The second set of primers is: hST6GN1-F2, ggaaggcacccgggac (SEQ ID NO:51) and hST6GN1-F1, ccGAATTCcggtcagttcttggct (SEQ ID NO: 52) (capital letters represent the restriction sites BamH I and EcoR I for cloning into pcDNA3.1, and the underlined residues indicate the XmaI site in the cDNA for putting the two pieces together).

The N-terminal fragment of hST6GalNAcI was amplified using clone #4816713 DNA as template, the first set of primers discussed above and Pfu DNA polymerase. The C-terminal fragment of hST6GalNAcI was amplified using clone# 6300955 as template, the second set of primers and pfu DNA polymerase. The PCR fragments were gel-purified using QIAEX II gel purification kit (Qiagen, Valencia, Calif.). Both DNA fragments were cloned into pCR-Blunt vector (Invitrogen, Zero Blunt PCR Cloning Kit, Carlsbad, Calif.). EcoR I digestions showed that both pCR-hST6-N#1-6 and pCR-hST6-C#1-6 have correct insert size.

pCR-hST6GalNAcI-N#1 and pCR-hST6GalNAcI-C#1 were digested with BamH I and Xma I, and Xma I and EcoR I, respectively. The released fragments were ligated with pcDNA3.1 (+) cut with EcoRI and BamHI. The final product pcDNA3.1 (+)-hST6GalNAcI-N1C1#1 was confirmed by both enzymatic digestions and DNA sequencing analysis. The obtained hST6GalNAcI cDNA has three nucleotide changes and two of them change the amino acid sequences (Q65K and M3791) These differences all originated from the EST clones

Three additional primers were designed to generate 3 truncations of hST6GalNAcI for expression in Sf9 cells. The primers are: bST6-K36-5′, ccaGGATCCaaggagcctcaaac (SEQ ID NO:53), hST6-K125-5′, ccaGGATCCaagagcccagaaaaagag (SEQ ID NO:54), and hST6-S258-5′, ccaGGATCCtctgagcctcggtgg (SEQ ID NO:55) (capital letters represent the restriction site BamH I for cloning into pAcgp67B). The K36 clone is truncated immediately after the transmembrane domain of human ST6GalNAcI and the S258 clone is truncated at the same relative position as the chicken ST6GalNAcI T233, according to an amino acid sequence comparison. The latter is the same published truncation used for chicken ST6GalNAcI expression in Sf9 (Kurosawa, N., et al (1994) J. Biol. Chem. 269, 1402-1409).

Three PCR products were obtained using the three primers paired with hST6GN1-R1, pcDNA3.1 (+)-hST6GalNAcI-N1C1# 1 as template and pfu DNA polymerase. All were cloned into pCR-blunt. K36#6, K125#4 and S258#6 sequence analysis confirmed that the vectors contained the correct cDNAs. The inserts from the pCR-blunt vector were cloned into the BamHI and EcoRI sites of pAcgp67B in-frame with the gp67 signal sequences. The sequences of the three trunctations, pAcgp67B-K36#4, K125#4 and S258#2 were confirmed by DNA analysis and were identified as the same as the full length human ST6GalNAcI sequences.

The DNA of above three truncated hST6GalNAcI in pAcgp67B, K36, K125 and S258, were co-transfected with BaculoGold DNA using BD BaculoGold Transfection Kit (BD Bioscience, Franklin Lakes, N.J.). To amplify the baculovirus, 0.1 ml of the transfection supernatant was used to infect 10 ml of Sf9 cells at 2×10⁶ cells/ml in a 10-cm dish. The P1 supernatant was harvested 3 days after infection. P2 viral stock was obtained by infecting 50-ml Sf9 cells at 2×10⁶ cells/ml and MOI=0.2. The baculovirus supernatants were amplified twice to get high titers. The virus titers were determined by BacPAK Baculovirus Rapid Titer Kit (BD Bioscience, Franklin Lakes, N.J.). A 50-ml scale production was set up at MOI=2, 2×10⁶ cells/ml. The culture supernatants were obtained at day 2-4. A ST6GalNAcI assay showed that both K36 and K125 expressed at 0.25-0.35 U/liter and S258 at 0.1-0.2 U/liter at 50-ml scale. Twelve plaque-purified K36 clones were further tested and amplified. Clone# 10 demonstrated the highest activity (1 U/liter). 1-liter scale production of clone# 10 had an expression level at 3 U/liter

pTS103 DNA (10 μg) was transformed into TOP10 cells and DNA was subsequently prepared from single colonies using a QIAprep Spin Miniprep Kit (Qiagen, Valencia, Calif.). pTS103 was analyzed by DNA sequencing analysis and the data demonstrated that this clone has several nucleotide differences from the published sequences. pTS103 is pAcgp67A with mouse ST6GalNAcI (mST6GalNAcI) having a K3 truncation and a myc tag at the end of C-terminus in between BamH I and BgI It restriction sites.

Primers were designed for making truncated mST6GalNAcI: S127 and S186. The primers were: S127-EcoRI-5′, cgGAATTCtctcagaacacctggac (SEQ ID NO:56), S186-EcoRI-5′, cgGAATTCtctctgagcctcggtgg (SEQ ID NO:57, mST6-XhoI-3′, gcCTCGAGtcagttctttgctttgtc (SEQ ID NO 58) (Capital letters represent the restriction sites for EcoR I and XhoI). The cloning vector used was pFastBac-1-gp, from Invitrogen (Carlsbad, Calif.), and a gp67 signal sequence was inserted between BamH I and EcoR I sites.

Two PCR products were obtained using the three above-referenced primers. pfu DNA polymerase and pTS103 were used as a template and cloned into pCR-blunt. Sequence analysis confirmed that pCR-S127#2 and S186#2 contained the correct cDNAs. The inserts from the pCR-blunt vector were cloned into the EcoR I and Xho I sites of pFastBac-1-gp in-frame with the gp67 signal sequences. pFastBac-1-gp-S127#3 and S186#2 were confirmed by EcoRI and XhoI double digestions and DNA sequence analysis.

pFastBac-1-gp-S127#3 and S186#2 DNA were transformed into DH10Bac competent cells from the Bac-to-Bac Baculovirus Expression System (Invitrogen, Carlsbad, Calif.). 12 white colonies from each transformation were re-streaked on plates and 8 out of 12 were actually white in color. “Bacmid” DNA was isolated using P1, P2 and N3 buffers with QIAprep Spin Miniprep Kit, according to the protocol from the manual (Qiagen, Valencia, Calif.). PCR screening was conducted to detect the insert of mST6GalNAcI in the bacmid DNA using M13F and mST6-XhoI-3′ as primers and Taq DNA polymerase (Qiagen, Valencia, Calif.). All 8 clones from each construct have the correct inserts and they were the same as the pTS103 sequences.

Additional bacmid DNA of S127, clone #5 and 6, S186, clone#3 and 4 were isolated from the bacteria using S.N.A.P MidiPrep Kit (Invitrogen, Carlsbad, Calif.). The bacmid DNA was tranfected into Sf9 cells using Cellfectin (Invitrogen, Carlsbad, Calif.). The baculovirus supernatants were amplified once to obtain high titers. The virus titers were determined by BacPAK Baculovirus Rapid Titer Kit (BD Bioscience, Franklin Lakes, N.J.). A 50-ml scale production was set up at MOI=2, 2×10⁶ cells/mt. The culture supernatants were obtained at days 2-4. ST6GalNAcI assay showed that both S127 viral stocks produced higher activities at 0.15-0.25 u/liter at 50-ml scale than either S186 viral stocks. Twelve plaque-purified S127 clones were further tested and amplified. All clones demonstrated the same activity, but clone#4 had slightly higher activity (0.46 u/liter). One-liter scale production of clone#4 demonstrated an expression level of 1.7 u/liter.

The above work demonstrated that both human and mouse GalNAc α-2,6-sialyltransferases (ST6GalNAcI) can be expressed in Sf9 (insect) cells and that the enzymes were secreted into the culture medium, with an expression level of about 2-3 u/liter.

### Example 4

### Expression of chicken N-acetylgalactosamine-α2,6-sialyltransferase (ST6GalNAcI) in Sf9 Cells Using Recombinant Baculovirus

Chicken N-acetylgalactosamine-α2,6-sialyltransferase (ST6GalNAcI) was expressed in Spodoptera frugiperda (Sf9) cells using the baculovirus expression vector system. N-acetylgalactosamine-α2,6-sialyltransferase (ST6GalNAcI) transfers sialic acid from CMP-sialic acid by an α2,6 linkage onto the C-6 hydroxyl group of a N-acetylgalactosamine (GalNAc) residue.

This enzyme was produced by infecting cultures of Sf9 cells with recombinant baculovirus. An alternate non plaque-purified baculovirus stock of chicken ST6GalNAcI was also used, based on use of the alternate clone in the published literature. This alternate clone was previously thought to be truncated at amino acid T233, but N-terminal sequence analysis showed that an extra amino acid before T233 was introduced during cloning, and, therefore, the polypeptide produced by the alternate clone contains amino acid K (lysine) 232 from the full length ST6GalNAcI sequence. Therefore, the alternate clone is actually truncated at K232. This stock was plaque-purified, amplified, and subsequently used for experiments herein.

Described herein are experiments conducted to obtain baculoviral DNA from plaque-purified viral stocks of the chicken ST6GalNAcI for sequence analysis of the enzyme and the conditions used to produce the enzyme from these viral DNA stocks. In this study, the enzyme produced had an average expression level of 11.8 units/L when produced in I liter scale using the following conditions: MOI=5-10, 130 rpm, 27° C., total cell count of 3.5e⁹ cells-7e⁹ cells and 72 hours of incubation.

Baculovirus DNA was isolated according to the following protocol. To the concentrated virus stock was added 6 μl 0.5 M EDTA and 4.5 μl 1 M Tris-HCl, pH 8.0. Then, 0.3 ml lysis buffer (0.2 M NaOH, 1% SDS) was added and the mixture incubated at room temperature for 5 minutes. After lysis, 0.3 ml of neutralization buffer (3M NaOAc, pH 5.2) was added and the mixture was incubated at 4° C. for 10 minutes. The mixture was clarified by centrifugation at 14,000 rpm for 10 minutes, at 4° C., in a microcentrifuge. The baculovirus DNA in the resulting 0.84 ml supernatant was precipitated using 0.8 ml isopropanol and incubated on ice for 10 minutes. The precipitated virus DNA was collected by centrifugation at 14,000 rpm for 10 minutes at room temperature. The resultant DNA pellet was washed with 0.5 ml 70% ethanol and air dried. The DNA pellet was then dissolved in 20 μl 1×TE Buffer (10 mM Tris-HCl, 1 mM EDTA, pH 8.0). DNA concentration was measured using OD260. The OD₂₆₀ 0.012, and therefore, DNA concentration=600 μg/ml.

Isolation of the chicken ST6GalNAcI was conducted using PCR. The primers used included ch233BamHI2 5′-GAT TCG GGA TCC ACG GAG CCA CAG TGG GAT TTT G-3′ (SEQ ID NO:59) and ch233Xho13′-GAT CGC CTC GAG TCA GGA TCT CTG GTA GAG CTT C-3′(SEP ID NO:7). A PCR reaction was set up with the following components: 5 μl 10×PCR Buffer, 2 μl 10 mM dNTP, 1 μl 5′ primer (10 pmol/μl), 1 μl 3′ primer (10 pmol/μl), 2 μl DMSO, 1 μl DNA template, 0.5 μl Herculase enzyme (Stratagene, Carlsbad, Calif.), and 37.5 μl PCR grade H₂O. The PCR program conditions included cycles of 95° C., 3 minutes; 95° C., 45 sec; 42° C., 1 minute, 72° C. 1 minute for 5 cycles; 95° C., 45 sec; 57° C., 1 minute, 72° C. 1 minute for 35 cycles; 72° C., 10 minutes; 4° C. pause.

PCR products were isolated using a MinElute Gel Extraction Kit (Qiagen, Valencia, Calif.). The DNA was eluted in 20 μl 1×TE (10 mM Tris-HCl, 1 mM EDTA, pH 8.0). pCRBlunt ligation and transformation was conducted using 4 ti of the PCR reaction product, 1 μl salt solution, and 1 μl TOPO pCR4 Blunt vector (ZeroBlunt TOPO, Invitrogen, Carlsbad, Calif.). A volume of 6 μl of the ligation mixture was then added to 50 μl of Top10 cells. The following ligation incubations were performed: First, on ice for 30 minutes, at 37° C. for 1 minute, then, on ice for 2 minutes. Reactions were conducted by adding 0.5 ml SOC medium, then incubating the mixture at 37° C. for 1 hour. After incubation, 200 μl of the mixture was plated on a Kanamycin-containing plate. About 100 colonies were generated.

Direct cloning of the PCR products was also carried out under the following conditions. A reaction mixture included 16 μl PCR product, 1 μl BamHI, 1 μl XhoI, 4 μl BamHI Buffer, 20 μl H₂O. The reaction mixture was incubated at 37° C. for 2 hours. Another reaction mixture included 1 μl pCWIN2-MBP vector (0.35 mg/ml), 0.5 μl BamHI, 0.5 μl XhoI, 2 μl BamHI buffer, and 16 μl H₂O. The reaction mixture was incubated at 37° C. for 2 hours. After gel electrophoresis of the direct cloning products, gel extraction, ligation and transformation, 10 colonies were selected and grown for plasmid DNA minipreps. Out of these 10 minipreps, 6 contained the correct insert in a pCWIN2-MBP vector.

Chicken ST6GalNAcI truncated at amino acid 232 was expressed and produced in Sf9 cells at a 1 liter scale using recombinant baculovirus, using conditions including a 1 liter scale, MOI=5-10, 130 rpm, 27° C., total cell count of 3.5e⁹ cells-7e⁹ cells and 72 hours of incubation time. The average expression level of the enzyme in these production runs is 11.8 Units/L.

Cells were counted using the Hemacytometer Method, and a working solution of trypan blue was prepared. Trypan blue is initially a 0.4% solution and it is diluted with PBS to a working concentration of 0.04% (1:10 dilution). A sample of cell suspension was aseptically withdrawn to be counted and dilutions (1:2, 1:4, 1:5, 1:10, 1:20) were prepared, as necessary, in the trypan blue working solution. Cells were counted within 3 minutes after being stained with trypan blue. Approximately 10 μl of the stained cell suspension was withdrawn and the tip of the pipet was placed onto the slot of a clean hemacytometer with coverslip. The cell suspension passed under the coverslip by capillary action. The hemacytometer was placed on the stage of an inverted microscope and read. The viable cell count was determined by using the equation: Viable Cell (Cells/ml)=(Number of Viable Cells Counted)/(Number of Squares Counted)×104× Dilution Factor. That is, the total viable cell number in the original suspension was found by multiplying the viable cells/ml by the total ml in the original suspension.

A plaque purification assay was then used. The method included counting Sf9 cells and determining viability, as described above. Cells must be at least 90% viable and in log phase growth. Cells were diluted with fresh media to a density of 5e⁵ cells/ml with a final volume between 20 and 30 ml. A volume of 2.0 ml of the cell suspension was added to each well in two 6 well plates and cells were rocked to distribute cells evenly. Each well contained approximately 1e⁶ cells. Plates were placed in a sealed container containing 2 paper towels dampened with approximately 50-100 ml of water to provide humidity. Plates were then placed on a rack on top of the towels to prevent direct contact with the wet towels, and were incubated in the container at 27° C. for 1-4 hours until the cells adhered to the bottom of the wells. Serial dilutions of 1:10 of the virus stock were made, from 1.0 e- to 1.0 e⁻⁹. A volume of 0-5 ml virus stock was placed into 4.5 ml SFM Sf-900 II media for dilution of the stock.

When the cells formed an even monolayer of about 70-80% confluency, the media was aspirated from the cells using a sterile pipette. A negative control was prepared by gently adding 1 ml of fresh media to each of two wells. Two wells for each dilution were infected, from 1.0 e⁻² to 1.0 e⁻⁹, by gently adding 1 ml of the virus dilution to each well. The plates were incubated at room temperature for 1 hour on a level surface to allow the virus to infect the cells. Plaquing medium was then prepared in a sterile 100 ml bottle, containing 30 ml of Sf-900 II 1.3× in 10 ml of 4% agarose. The bottle was incubated in a 37° C. waterbath until ready to use (after 1 hour viral incubation). After the 1 hour incubation, the virus inoculum was aspirated from the cells using a sterile pipette by tilting the plate and aspirating from the edge. 2.0 ml of plaquing medium was added to each well. The agarose was allowed to set for 10-15 minutes at room temperature, then the preparations were incubated at 27° C. in the sealed container with wet paper towels for 5 to 7 days, until the plaque appeared.

Plaque purification was conducted by picking a plate with plaques that were spaced far apart. Using a sterile Pasteur pipet and bulb, a clear plaque was picked and transferred, via agarose plug (containing virus), to a sterile 1.5 ml microcentrifuge tube containing 500 μl SFM Sf-900 II media. The agarose plugs were incubated in media at 4° C. overnight. Virus was amplified to Passage 1 (P=1) amplification.

Six-well plates were seeded with log-phase Sf9 cells at 7e⁵ cells/ml in 3 mls (˜2.0e⁶ cells total/well) and allowed to settle for 5-15 minutes at room temperature. Plates were infected with 100 μplaque “pick-up” and shaken gently. One well with no infection was used as a negative control. Plates were incubate at 27° C. for 3-4 days, until observation of signs of infection (grainy-looking, shriveling, dying cells). Supernatant was harvested and assayed for protein. The well containing the highest activity for further amplification was the P=1 virus stock.

Asialo Bovine Submaxillary Mucin (asialo BSM) or asialo Ovine Submaxillary Mucin (asialo OSM) substrate was prepared for a ST6GalNAcI enzyme assay. Sialic acid was released by hydrolysis, in a reaction containing 500 μl BSM or OSM (20 mg/ml), 500 μl dH₂O, and 130 μl 2 M glacial acetic acid. Components were mixed and incubated at 80° C. for 5 hours to 18 hours. The reaction mixture was diluted with 5 ml PBS. Samples were loaded onto Amicon Ultra-15 columns and centrifuged at 3,000×g 4° C. for 20 minutes (Millipore, Bedford, Mass.). Five ml of PBS was added and the columns centrifuged again. The process was repeated three times, or until the mixture was at approximately pH 7.0. Untreated BSM or OSM was used to prepare a standard curve to estimate the concentration of AOSM or ABSM by linear regression.

A radioactive assay was used to assay ST6GalNAcI. The reaction mixture included CMP 14C sialic acid (dried down by nitrogen) at a concentration of 100,000 CPM, cold CMP sialic acid at 0.2 mM (10 nmoles total in reaction), A-BSM (acceptor substrate, 0.25 mg), MES pH 6.0 at 50 mM, and NaCl at 100 mM, with 10 μl of enzyme sample in a total of 40 μl reaction volume. Enzyme-free and/or acceptor-free negative control(s) were included. The reaction mixtures were incubated at 37° C. for 1 hour, at which point, 100 μl (per reaction) of 5% phosphotungstic acid/15% TCA was added and mixed well. The sample was prepared by centrifugation at maximal speed in microfuge for 2 minutes and the supernatant discarded. TCA (5%) was added at 500 μl per reaction and the sample vortexed. The sample was again centrifuged at maximal speed in a microfuge for 2 minutes, the supernatant discarded by pipetting. Pellets were resuspended in 100 μl 10N NaOH, 1 ml water was added, and 5 ml scintillation fluid was added to the resulting mixture, and the mixture counted for 1 minute.

TABLE 14
Calculations for ST6GalNAcI activity in Units/Liter.

Unit = transfer of 1 μmol of CMP Sialic Acid/minute
U/L = [(cpm corr) (DF) (10 nmoles CMP sialic acid) (1 umol) (1000 μl)
(1000 ml) (5.5 conversion factor)]/[(total cpm corr) (60 min) (10 μl sample
volume) (1000 nmol) (1 ml) (L)]
background cpm = cpm of sample with no enzyme or no acceptor
cpm corr = cpm minus background cpm
total cpm corr = total cpm minus background cpm
Conversion factor = Factor for working at a acceptor substrate
concentration less than the Km as determined by previous related work.

Passage 2 viral amplification was conducted by growing suspension of Sf9 cells to a concentration of 2.0 e⁶ cells/ml in 250 ml disposable erlenmeyer flask, which contained 30 ml to 50 ml of SFM Sf-900 II media Titered viral stock was added at an MOI of 0.2, and fresh SFM Sf901 media was added to a total volume of 50 ml to 100 ml. The cultures were incubated in shaking incubator for 48 hours, at 27° C., 130 rpm. Cells were harvested by centrifugation using sterile 250 ml conical centrifuge tubes. The viral stock was titred by end point dilution assay.

Large scale virus stock was prepared in 59 cells. A suspension of S59 cells was grown to a concentration of 7.0 e⁶ cells/ml to 1.4 e⁷ cells/ml (3.5e⁹ to 7e⁹ total cells) in a 2 L non-baffled fernbach flask containing 500 ml of SFM Sf-900 II media. Titered viral stock was added at an MOI of 0.2, and fresh SFM Sf900II media was added to a total volume of 1 liter. The cultures were incubated in a shaking incubator for 48 hours, 27° C., 130 rpm, and the cells harvested by centrifugation using sterile 1 L centrifuge bottles. The viral stock was titred by an end point dilution assay and stored at 4° C.

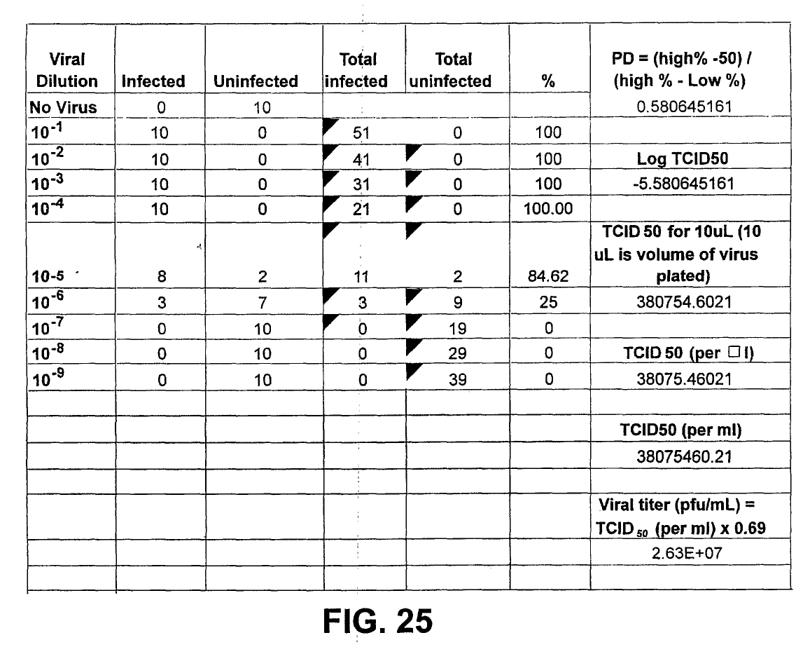

Viral stocks were also titred using and end point dilution assay as follows. Cells were counted and viability determined as described above. Cells were at least 90% viable and in log phase growth. Cells were diluted with fresh media to a density of 2.5e⁵ cells/ml in 10 ml and cells were then plated at 10 μl/well in 72-well microtiter plate. Media was plated only in the last 2 wells of each row. Serial (1:10) dilutions of virus stock from 1.0 e⁻¹ to 1.0 e⁻⁹. Virus stock (100 μl) was placed into 900 μl SFM Sf-900 II media for dilution (1.0 ml volume total dilution), and 10 μl of the 1.0 e⁻¹ diluted stock was placed into each of 10 wells of the first plate. Plates were incubated at 27° C. for 7 days in a humid container. The plates were observed using a microscope with a 10× objective. Wells were scored as “infected” or “not infected.” The Reed-Muench formula (Reed, L. J., and Muench, H. (1938), Amer. Jour. Hygiene, 27, 493-497.) was used to determine 50% infectivity dose (TCID₅₀) of virus is used to determine viral titer. FIG. 25 illustrates the titer determination worksheet used as described above.

Large scale protein ST6GalNAcI production in Sf9 Cells included growing a suspension of Sf9 cells to a concentration of between 7.0 e⁶ cells/ml to 1.4 e⁷ cells/ml (3.5e⁹ to 7e⁹ total cells) in 2 L non-baffled fernbach flask containing 500 ml of SFM Sf900II media. Titered viral stock was added to the culture at an MOI of 5-10. Fresh SFM Sf900II media was then added to a total volume of 1 liter, and the cultures incubated in shaking incubator for 72 hours, at 27° C., 130 rpm. Cells were harvested by centrifugation using sterile 1 Liter centrifuge bottles. The resultant supernatant was filtered through a 0.21 um filter unit and the final product stored at 4° C.

TABLE 15
ST6GalNAcI activity of screening plaque-purified P = 1 viral stocks

| | | Corrected | | ST6GalNAcI |
|---|---|---|---|---|
| Sample | Sample cpm | Sample cpm | DF | activity U/L |
| Blank (NaOH only) | 10 | — | 1 | — |
| Blank (no enzyme) | 23 | — | 1 | — |
| Blank (media only) | 23 | — | 1 | — |
| Blank (no acceptor) | 21 | — | 1 | — |
| ch-ST6GalNAcI pure | 9744 | 9724.75 | 10 | 231.523 |
| ch-P1 Clone #1 | 42 | 22.75 | 1 | 0.054 |
| ch-P1 Clone #2 | 121 | 101.75 | 1 | 0.242 |
| ch-P1 Clone #3 | 62 | 42.75 | 1 | 0.102 |
| ch-P1 Clone #4 | 168 | 148.75 | 1 | 0.354 |
| ch-P1 Clone #5 | 121 | 101.75 | 1 | 0.242 |
| ch-P1 Clone #6 | 67 | 47.75 | 1 | 0.114 |
| ch-P1 Clone #7 | 153 | 133.75 | 1 | 0.318 |
| ch-P1 Clone #8 | 116 | 96.75 | 1 | 0.230 |
| ch-P1 Clone #9 | 71 | 51.75 | 1 | 0.123 |
| ch-P1 Clone #10 | 158 | 138.75 | 1 | 0.330 |
| ch-P1 Clone #12 | 55 | 35.75 | 1 | 0.085 |
| ch-P1 Clone #13 | 69 | 49.75 | 1 | 0.118 |
| ch-P1 Clone #14 | 75 | 55.75 | 1 | 0.133 |
| ch-P1 Clone #15 | 61 | 41.75 | 1 | 0.099 |
| ch-P1 Clone #16 | 49 | 29.75 | 1 | 0.071 |
| Average blank cpm | 19.25 | | | |
| Total cpm | 50834 | | | |

The purpose of screening the plaque-purified P=1 viral stocks is to identify a single clonal isolate containing enzyme activity. Clone 4 (0.354 U/L), Clone 6 (0.318 U/L), and Clone 10 (0.330 U/L) had the highest activities and are good candidates for further amplification. Clone 4 was chosen since it had the highest activity of the three.

TABLE 16
Large-scale production of chicken ST6GalNAcI

| | | Total cell | | | Activity |
|---|---|---|---|---|---|
| | | density | Production | Harvest | (Units/ |
| Production Lot # | MOI | at infection | Scale (L) | Time (hrs) | Liter) |
| 4-081503-1LP1 | 10 | 4.5e9 cells | 1 | 72 | 9 |
| 4-81903-1LP2 | 10 | 3.6e9 cells | 1 | 96 | 0 |
| 4-82603-1LP3 | 10 | 5.1e9 cells | 1 | 72 | 9 |
| 4-82803-1LP4 | 5 | 5.5e9 cells | 1 | 72 | 8 |
| 4-90203-1LP5 | 8.3 | 4.6e9 cells | 1 | 72 | 12 |
| 4-90903-1LP6 | 10 | 7e9 cells | 1 | 96 | 2 |
| 4-91603-1LP7 | 10 | 5.5e9 cells | 1 | 72 | 10 |
| 492203-1LP8 | 10 | 3.5e9 cells | 1 | 72 | 23 |

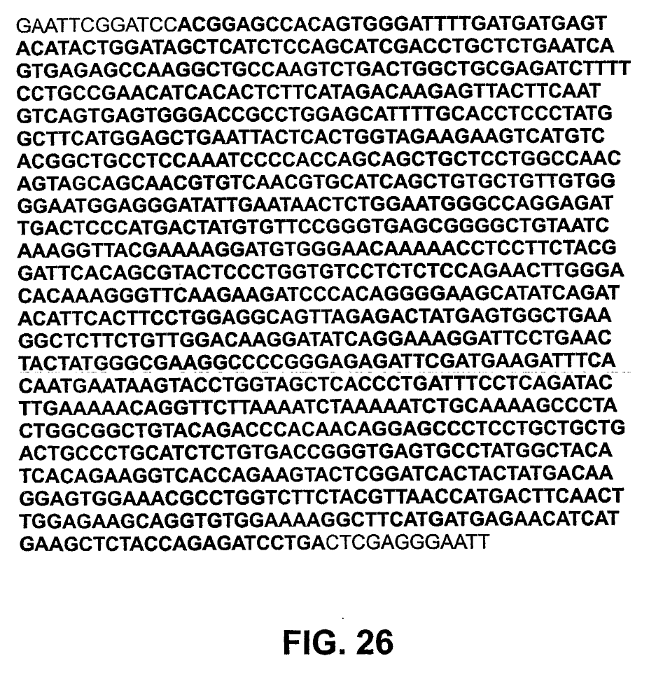

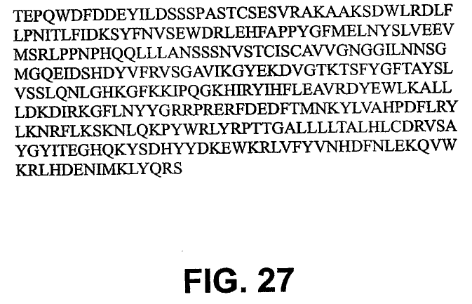

The sequence of Chicken ST6GalNAcI was confirmed as follows. N-terminal sequencing was conducted using 20 ug of purified chicken ST6GalNAcI, resulting in the sequence: VSTEDPKTEPOWDFDDEYILDSSS (SEQ ID NO:8), which verified that the chicken ST6GalNAcI used for the experiments described herein had the same amino acid sequence (underlined) as published X74946 chicken ST6GalNAcI truncated at amino acid K232. DNA sequencing of the chicken ST6GalNAcI was conducted using 50 ml of chicken ST6GalNAcI baculovirus stock. Viral DNA was extracted from this stock, PCR-amplified, inserted into the vector pCWIN2-MBP, and sequenced. DNA was sequenced from the point of the T233 truncation, not the K232 truncation. The resulting DNA had Sac2/Kpn2 restriction sites, and had 1029 bases with a 49.36% GC content (FIG. 26). Translation of the sequence obtained, shown in FIG. 27, revealed a one residue difference when compared to published chicken ST6GalNAcI GenBank X74946, namely, V25 IA (GTA to GCA, valine to alanine). The experimental DNA sequence had one other mutation, a silent mutation T233 (ACT to ACG, same amino acid, threonine) in pCWIN2-MBP-chST6GalNAc, which was introduced by a PCR primer during cloning.

K232 was not included in when viral DNA was PCR amplified. The rest of the DNA sequence was verified to be the same as the published sequence.

In summary, chicken ST6GalNAcI viral stock was plaque-purified, amplified, and enzyme was produced from the stocks. Eight production runs were done at a 1 liter scale. Two of the runs that were infected for 96 hours had little or no activity. The best conditions seen for the production runs performed during the time of this report are MOI=5-10, 130 rpm, 27° C., total cell count of 3.5e⁹ cells to 7e⁹ cells, and 72 hours of incubation. Under these conditions, the average activity of the produced ST6GalNAcI was 11.8 units/liter. The chicken ST6GalNAcI sequence was also verified N-terminal sequencing was performed on purified chicken ST6GalNAcI protein and sequence analysis confirmed that it was truncated at E32 and had the same amino acids in the N-terminal portion as the published sequence DNA sequencing was also performed for verification of sequence. Recombinant viral DNA was extracted from chicken ST6GalNAcI baculovirus stock and PCR amplified. The DNA was PCR amplified from the T233 truncation and not K232. The DNA was inserted into vector pCWIN2-MBP and sequenced. Results revealed one base difference (GTA to GCA) in the sequenced chicken ST6GalNAcI as compared to the published sequence GenBank X74946. This difference results in a one amino acid difference of V251A (valine to alanine) in the polypeptide. The DNA sequence also revealed one other silent mutation T233 (ACT to ACG) which was introduced by PCR primer. The rest of the DNA sequence was confirmed to be the same as the published sequence.

### Example 5

### Sialyltransferase Activity of N-Terminal Deletions of Chicken N-acetylgalactosamine-α2,6-sialytransferase (ST6GalNacI) in Sf9 Cells Using Recombinant Baculovirus

This example describes the expression of four N-terminal deletions of chicken N-acetylgalactosamine-α2,6-sialyltransferase (ST6GalNAcI), in Spodoptera frugiperda (Sf9) cells, using a pAcGP67 baculovirus expression vector system. N-acetylgalactosamine-α2,6-sialyltransferase (ST6GalNAc I) transfers sialic acid from CMP-sialic acid, by an α2,6 linkage, onto a N-acetylgalactosamine (GalNAc) residue, O-linked to a threonine or serine of a glycoprotein.

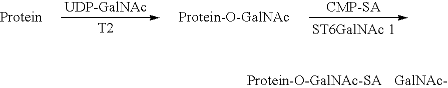

A viral stock expressing an N-terminal deletion of chicken ST6GalNAcI was obtained. This viral stock was produced using a pVL1392 baculovirus expression system (Blixt et al., 2002, J. Am. Chem. Soc., 124:5739-5746). The enzyme activity of multiple 10×1 L enzyme production runs using this viral stock averaged 12 U/L.

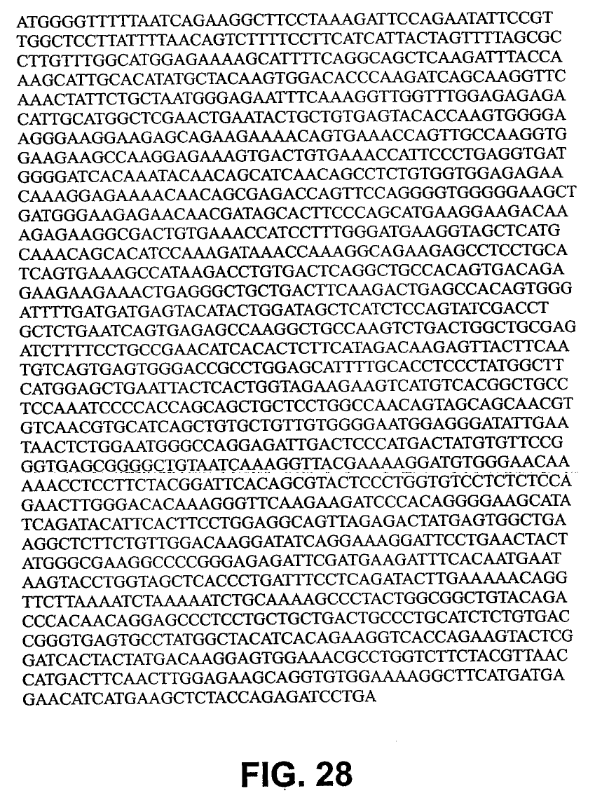

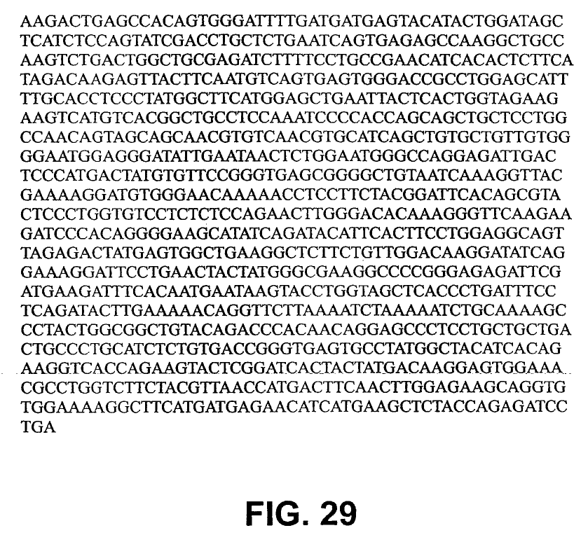

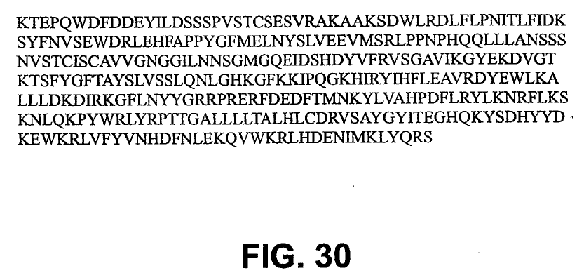

Four N-terminal deletions of chicken ST6GalNAcI-Δ48, a truncation mutant that begins at amino acid 49 of the full-length chicken ST6GalNAcI; A152, a truncation mutant that begins at residue 153 of the full-length chicken ST6GalNAcI; A225, a truncation mutant that begins at residue 226 of the full-length chicken ST6GalNAcI; and A232, a truncation mutant that begins at residue 233 of the full-length chicken ST6GalNAcI—were created using PCR. The resultant four PCR fragments contained ST6GalNAcI coding sequences beginning with amino acids Q49, V153, L226 and T233, respectively. Sites of N-terminal deletions of the chicken ST6GalNAcI were chosen based upon sequence similarities among the human, mouse and chicken ST6GalNAcI coding sequences (FIG. 28).

The Δ48 N-terminal deletion deletion mutant was designed to create a coding sequence initiating immediately after the predicted transmembrane domain. The transmembrane region of chicken ST6GalNAcI had previously been predicted to be between amino acids 17 to 37 (Kurosawa et al., 1994, J. Biol. Chem., 269:1402-1409), but a hydropathy plot analysis suggested a transmembrane region between amino acids 26 and 48. The Δ152 N-terminal deletion mutant was selected to create a truncation mutant that included the portion of the stem region of chicken ST6GalNAcI enzyme that contained predicted areas of sequence similarity with the human and mouse enzymes (FIG. 31). The third N-terminal deletion mutant, Δ232, was created to resemble the ST6GalNAcI coding sequence as published by Blixt et al. (2002, J. Am. Chem. Soc., 124:5739-5746).

Initial activity assays indicated the Δ232 viral stock was inactive (see below). The ST6GalNAcI sequence contained in the original viral stock was therefore analyzed. It was determined that additional N-terminal amino acids identical to those present in the wild type enzyme were inadvertently donated to this truncation mutant sequence from the multiple cloning site of the vector, which included insertion of the amino acids DPK immediately N-terminal to Δ232. In the ST6GalNAcI family, the amino acids immediately upstream of Δ232 in all three sequences is (NED)FK (see Appendix 2). Therefore, a fourth N-terminal deletion, Δ225, was created to be representative of the clone described by Blixt et al. (2002, J. Am. Chem. Soc., 124:5739-5746).

A chicken ST6GalNAcI viral stock was produced using a vector, pVL1392, that contained a dog insulin secretion signal peptide. Other deletions prepared for this study were cloned into a pAcGP67B vector (Pharmingen, San Diego, Calif.), which contains the glycoprotein 67 (gp67) secretion signal peptide. The gp67 signal peptide was used as a stronger secretion signal than the dog insulin secretion peptide. PCR reactions were set up as illustrated in Table 17.

TABLE 17
PCR Reactions for generation of truncation mutants.

| | |
|---|---|
| 5 μl | 10x PCR Buffer |
| 2 μl | 10 mM dNTP |
| 1 μl | 5′ primer (10 pmol/μl) |
| 1 μl | 3′ primer (10 pmol/μl) |
| 2 μl | DMSO |
| 1 μl | DNA template (10 ng/μl) |
| 0.5 μl | Herculase (Stratagene, Cat # 600260-51, Lot # 1220210) |
| 37.5 μl | PCR grade H₂O |

The PCR program was conducted under the following cycles: a) 95° C. 3 minutes; b) 95° C., 45 sec; 42° C. 1 minute, 72° C. 1.5 minutes for 5 cycles; c) 95° C., 45 sec; 57° C. 1 minute, 72° C. 1.5 minutes for 30 cycles; d) 72° C. 10 minutes; e) 4° C. pause.

The PCR primer pair used to generate the Δ232 mutant was ch233BamHI2, 5′-GATTCGGGATCCACGGAGCCACAGTGGGATTTTG-3′ (SEQ ID NO:60) and ch233XhoI, 5′-GATCGCCTCGAGTCAGGATCTCTGGTAGAGCTTC-3 (SEQ ID NO:61). Isolated and concentrated baculovirus DNA template was used for PCR. One microliter of template (600 ng/μl) was used for PCR. A 1002 bp PCR product was produced.

The PCR primer pair used to generate Δ48 was Δ48BamHI, 5′-GGATCCCAAAGTATTGCACACATGCTACAAG-3′ (SEQ ID NO:62) and S566EcoRI, 5′-GGCGAATTCTCACGATCTCTGGTAGAGTTTC-3′ (SEQ ID NO:63). The PCR primer pair used to generate the A 152 mutant was Δ152BamHI, 5′-GGATCCGTTCCAGGTGTGGGAGAAGC-3′ (SEQ ID NO:64) and S566EcoRI (SEQ ID NO:63). The DNA template for both PCR fragments was plasmid DNA pBluescript-chST6GalNAcI. For chST6GalNAc Δ48, a 1554 bp PCR product was produced. For chST6GalNAc 1-Δ152, a 1242 bp PCR product was produced.

The PCR primer pair used to generate the Δ225 mutant was A225BamHI, 5′-GGATCCCTGAGGGCTGCTGACTTCAAGAC-3′ (SEQ ID NO:65) and 5′-GGTGCTTAAGAGTAATGCTAGAGACCATCTCAAAGTAC-3′ (SEQ ID NO:66). The DNA template was plasmid DNA pBluescript-chST6GalNAcI. The annealing temperature for the first 5 cycles was 40° C. and for the last 30 cycles was 53° C. For chST6GalNAc 1-Δ225, a 1023 bp PCR product was produced.

The PCR bands were electrophoresed and isolated by gel extraction. The DNA was eluted in 20 μl 1×TE (10 mM Tris-HCl, 0.1 mM EDTA, pH 7.5). To ligate the isolated PCR products into a vector, ligation reactions were conducted with each isolated DNA. For the chST6GalNAcI-Δ232 PCR product, the ligation reaction contained 4 μl PCR product, 1 μl salt solution, and 1 μl TOPO pCR4 (Invitrogen, Carlsbad, Calif.). The reaction was incubated at room temperature for 15 minutes. For all other PCR products, the ligation reactions contained 4-7 μl PCR product, 1 μl pCR4 Blunt vector (Invitrogen, Carlsbad, Calif.), 1 μl T4 DNA ligase Buffer, 1 μl T4 DNA ligase, with the remaining volume up to 10 μl comprising H₂O. The ligation reactions were incubated at 16° C. for 1 hour.

Subsequently, 6 μl of each ligation mixture was added to separate tubes containing 50 μl of Top10 cells (Invitrogen, Carlsbad, Calif.). Incubations of each were performed on ice for 30 minutes, at 37° C. for 1 minute, on ice for 2 minutes, adding 0-5 ml SOC then 37° C. 1 hour. After incubation, 200 μl of each incubation mixture was spread on kanamycin-containing plate. Approximately 100 colonies were generated for each transformation reaction.

Single colonies were selected and grown overnight at 37° C. in 3 ml medium containing 50 μg/ml Kanamycin. The inserts were verified as being correct by using pairwise restriction enzymes corresponding to restriction sites designed into the PCR primers.

The pAcGP67B vector and each insert in the pCRBlunt vector were digested with the restriction site-appropriate, pairwise restriction enzymes. The digested DNA was separated on 0.8% agarose gets. The corresponding bands were excised with a surgical blade and DNA was extracted from the gel using a MiniFlute Kit (Qiagen, Valencia, Calif.). The insert and vector were ligated together using T4 DNA ligase (in ratios ranging from 1:1 to 6:1). The ligation mixtures were transformed into Top10 cells (Invitrogen, Carlsbad, Calif.) and spread on carbenicillin-containing plates. After overnight incubation at 37° C., several colonies were picked and screened for the correct insert and vector for each plasmid. Subcloning procedures included pAcGP67B-Δ232 BamHI/EcoRI, pAcGP67B-Δ152 and pAcGP67B-Δ48 BamHI/EcoRI and pAcGP67B-Δ225 BamHI/EcoRI.

In a 25 cm² Falcon flask (BD Bioscience, Franklin Lakes, N.J.), 1×10⁶ Sf9 cells were seeded (50 to 70% confluence). Linearized BaculoGold DNA (BD Bioscience, Franklin Lakes, N.J.) (0.5 μg) was mixed with 2 pg recombinant plasmid DNA and 100 μl of SF900 II SFM (Invitrogen, Carlsbad, Calif.) in a microfuge tube. In another tube, 6 μl of cellfectin was mixed with 100 μl SF900 II SFM (Invitrogen, Carlsbad, Calif.). The two mixtures were combined and incubated at room temperature for 15 to 45 minutes. The medium in the flask was removed and Sf9 cells were covered with the DNA mixture. An additional 0.8 ml of SF900 II SFM (Invitrogen, Carlsbad, Calif.) was added to the flask and incubated at 27° C. for 5 hours. After the incubation, the DNA mixture and cellfectin were removed and 3 ml of fresh SF900 II SFM (Invitrogen, Carlsbad, Calif.) was added to the flask. The Sf9 cells in the flask were incubated, without shaking, for 5 days at 27° C. Visible infection was observed after 72 hours.

Following a 5 day incubation, the culture supernatant was cleared by centrifugation at 1,000×g for 10 minutes. This supernatant was labeled the Passage 1 (P1) viral amplification stock. One ml of the P1 viral stock was incubated with a 50 ml suspension culture of Sf9 cells (2×10⁶ cells/ml). The incubation was conducted at 27° C., with stirring at 100 rpm for 5 days. The culture was harvested by centrifugation in a Corning sterile conical centrifuge tube (Corning, Corning, N.Y.) at 5000 rpm (7,000×g) for 30 minutes at 4° C. and the resultant supernatant was labeled the Passage 2 (P2) viral amplification stock.

Twelve ml of the P2 viral stock was incubated with a 150 ml suspension culture of Sf9 cells (2×10⁶ cells/ml). The incubation was conducted at 27° C., with stirring at 100 rpm for 5 days. The supernatant, isolated as described for the P2 viral stock, was labeled the Passage 3 (P3) viral amplification stock. P1 and P2 were stored at −80° C. P3 was stored at 4° C. in the dark. The titer of the recombinant baculovirus was determined by plaque assay.

Recombinant protein was produced by infecting 200 ml of 2×10⁶ cells/ml Sf9 cells with 25 ml of the P3 viral stock. The culture was incubated at 27° C., with stirring at 100 rpm for 72 hours. The supernatant was isolated as described for the P2 and P3 viral stocks.

The resultant supernatants were assayed for ability to catalyze sialylation of asialo bovine submaxillary mucin and to catalyze the transfer of a sialic acid-polyethylene glycol conjugate to G-CSF (“sialylPEGylation” of G-CSF). More generically, the design and transfer of a glycan-polyethylene glycol conjugate, or “glycoPEG” conjugate, to another molecule is presented at length in International (PCT) Patent Application No. WO03/031464 (PCT/US02/32263), which is incorporated herein by reference in its entirety.

Radioactive assays were used to measure the transfer of ¹⁴C-sialic acid from ¹⁴C-CMP-sialic acid to asialo-bovine submaxillary mucin, as described elsewhere herein.

TABLE 18
SialylPEGylation Assay

| | |
|---|---|
| G-CSF-O-GalNAc (0.4 mg/mL) | 5.00 μl |
| 125 mM MnCl₂ | 0.50 μl |
| CMP-SA-PEG-20K | 0.25 μl |
| Chicken ST6GalNAc 1 | 5.00 μl |
| 50 mM MES pH 6.0 | 1.25 μl |
| Total volume | 12.0 μl |

The sialylPEGylation reaction mixture was incubated at 33° C. with gentle shaking for 18 to 72 hours (as described below). After incubation, 2-5 III of 5×SDS Sample Buffer was added to each reaction mixture and the entire reaction mixture was subjected to electrophoresis in a 4-20% SDS-PAGE gradient gel. PEGylated G-CSF was detected by iodine staining of the gel.

Using a DNA miniprep analysis, the pCRBlunt constructs were examined for insert. The analysis demonstrated that pCRBlunt-chST6GalNAcI-Δ232 BamHI/XhoI colonies # 2 to 15 # 6 were identified as containing the correct insert, pCRBlunt-chST6GalNAcI-Δ152 BamHI/EcoRI colonies # 1., # 3 to # 6 were identified as containing the correct insert, pCRBlunt-chST6GalNAcI-Δ48 BamHI/EcoRI colonies # 1., # 2., # 3., # 5 and # 6 were identified as containing the correct insert, and pCRBlunt-chST6GalNAc 1-Δ225 EcoRI colonies A 2 were identified as containing the correct insert.

Using a DNA miniprep analysis, the subcloning constructs were examined for insert. The analysis demonstrated that pAcGP67B-chST6GalNAcI-Δ232 BamHI/EcoRI colonies # 1 to # 4 were identified as containing the correct insert, pAcGP67B-chST6GalNAc 1-Δ152 BamHI/EcoRI colonies # 1 to # 4 were identified as containing the correct insert, pAcGP67B-chST6GalNAcI-Δ48 BamHI/EcoRI colonies # 1 to # 4 were identified as containing the correct insert, and pAcGP67B-chST6GalNAc 1-Δ225 BamHI/EcoRI colonies # 1 to # 8 were identified as containing the correct insert.

The titers of recombinant baculovirus containing chicken ST6GalNAc l mutants were also determined. The Δ232 mutant had a titer of 8.50×10⁶, the Δ152 mutant had a titer of 2.28×10⁷, and the Δ48 mutant had a titer of 1.28×10⁷.

TABLE 19
Summary of Sialylation Activity in Radioactive Assay

| | Average | Average Activity (Units/L) |
|---|---|---|
| Samples | Activity (Units/L) | (1:5 sample dil) |
| Positive Control | 40.6 | 51.5 |
| K232 VS4-001 | 19.2** | 19** |
| Δ232 # 1 | 0 | ND |
| Δ232 # 2 | 0 | ND |
| Δ48 # 1 | 35.6** | 47** |
| Δ48 # 2 | 34.5** | 38.8** |
| Δ152 # 1 | 39.5** | 44.1** |
| Δ152 # 2 | 39.9** | 35.5** |
| Δ225 | 27.877** | ND |
| | (supernatant) | |

Purified enzyme, using K232 VS4-001, was used as a positive control. MOI used for protein production were as follows: Δ48 #1, 0.800; Δ152 #1, 1.430; Δ232 #1, 0.531; Δ48 #2, 0.200; Δ152 #2, 0.356; Δ232 #2, 0.133. RSD less than 2.5%

Note: For the Δ232 viral stocks, since the activities were zero on Apr. 8, 2004, they were not re-assayed on Apr. 26, 2004. Mutants with results marked with a double asterisk (**) tested positive for sialylPEGylation activity.

### Example 6

### Refolding of MBP-ST6GalNAcI Proteins

Eukaryotic ST6GalNAcI was fused to MPB. Briefly, five mouse ST6GalNAcI constructs were generated: D32, E52, S127, S186, and S201. Each construct was expressed behind the MBP-tag from the vector pcWin2-MBP, and differ in the extent of the ‘stem’ region included in the construct. D32 is the longest form, starting immediately downstream of the predicted amino-terminal transmembrane domain. S201 is the shortest, beginning shortly before the predicted start of the conserved catalytic domain.

In addition to the mouse constructs, human ST6GalNAcI K36 was also expressed as a fusion with MBP. The human construct begins just after the transmembrane domain. DNA encoding human ST6GalNAcI from K36 to its c-terminus was isolated by PCR using the existing baculovirus expression vector as template, and cloned into the BamHI-XhoI sites within pcWin2 MBP.

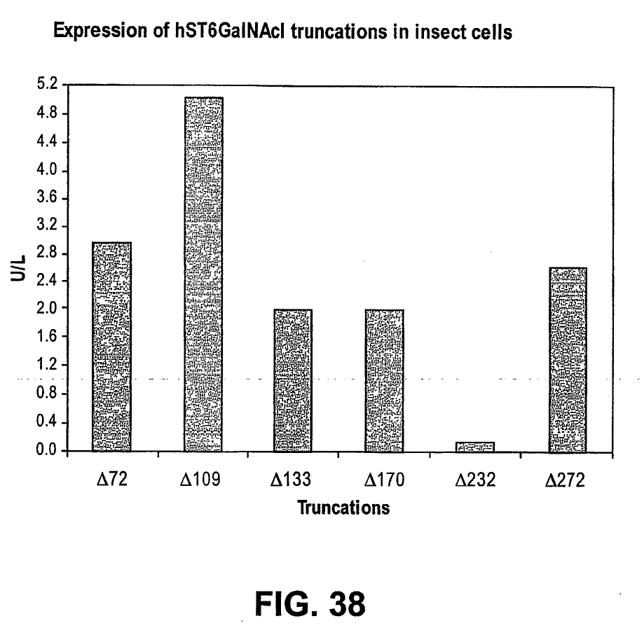

For reference, the sequences for MBP-mST6GalNAcI S127 and MBP-hST6GalNAcI K36 are included in FIG. 26. In addition, FIG. 38 provides full length amino acid sequences for human ST6GalNAcI and for chicken ST6GalNAcI, and a sequence of the mouse ST6GalNAcI protein beginning at residue 32 of the native mouse protein.

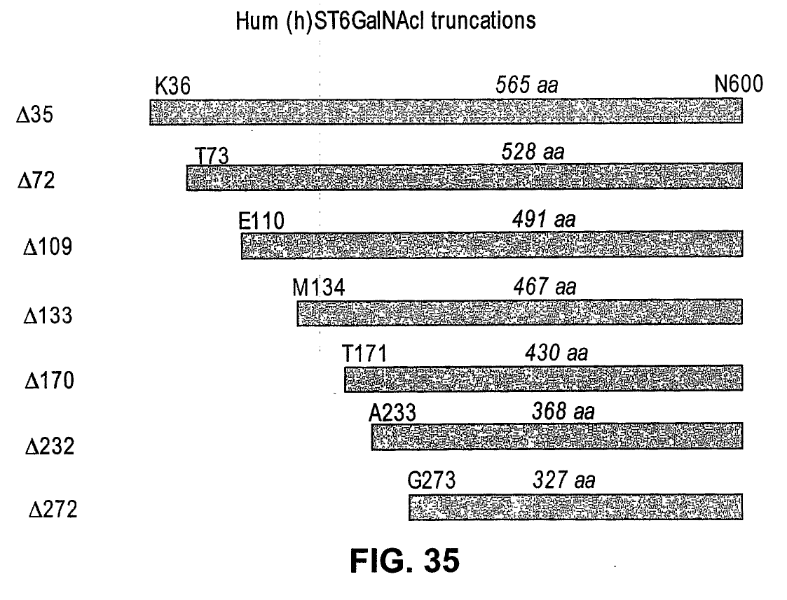

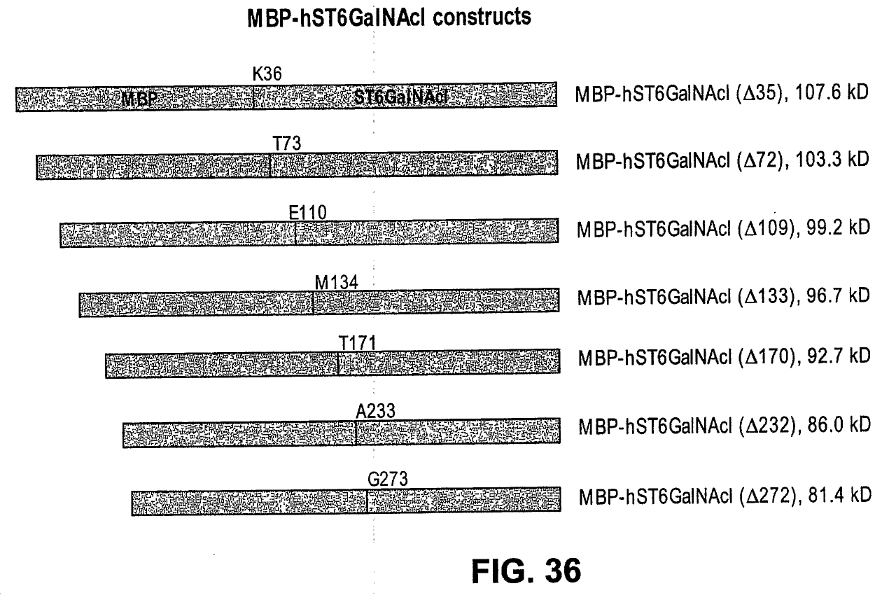

Deletion mutants additional to those described above have been made and a complete list of preferred ST6GalNAcI for use in the invention is found is Table 20. FIG. 35 provides a schematic of a number of preferred human ST6GalNAcI truncation mutants. FIG. 36 shows a schematic of MBP fusion proteins including the human ST6GalNAcI truncation mutants.

TABLE 20
ST6GalNAcI Mutants

| | Truncation Site | Mutation |
|---|---|---|
| HUMAN | Δ35 | K36 |
| | Δ124 | K125 |
| | Δ257 | S258 |
| | Δ35 | K36 |
| | Δ72 | T73 |
| | Δ109 | E110 |
| | Δ133 | M134 |
| | Δ170 | T171 |
| | Δ232 | A233 |
| | Δ272 | G273 |
| CHICKEN | Δ48 | Q49 |
| | Δ152 | V153 |
| | Δ225 | L226 |
| | Δ226 | R227 |
| | Δ232 | T233 |
| MOUSE | Δ30 | K31 |
| | Δ31 | D32 |
| | Δ51 | E52 |
| | Δ126 | S127 |
| | Δ185 | S186 |
| | Δ200 | S201 |

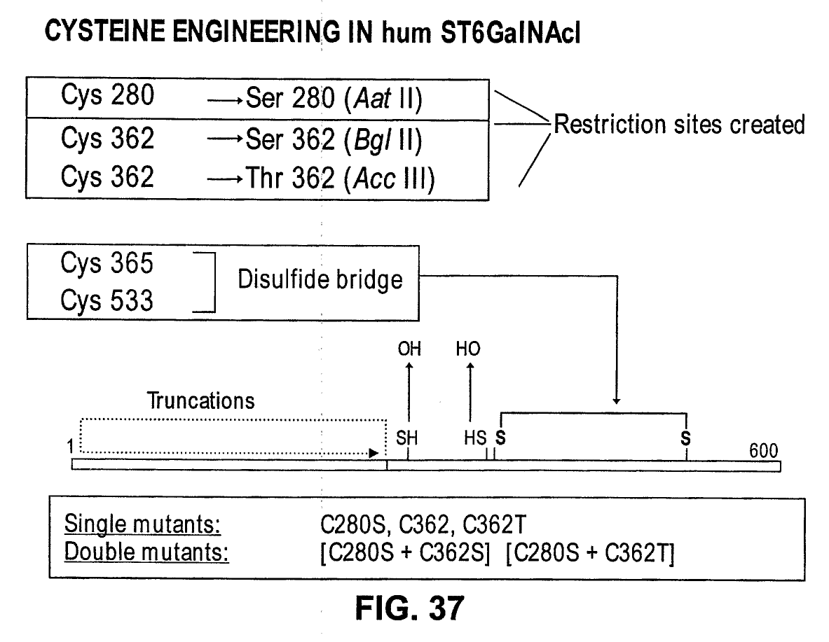

FIG. 37 shows the position of paired and unpaired cysteine residues in the human ST6GalNAcI protein. Single and double cysteine substitution are also shown, e.g., C280S, C362S, C362T, (C280S+C362S), and (C280S+C362T).

Initial expression studies showed that the ST6GalNAcI fusion proteins were expressed as insoluble proteins. To recover active recombinant enzyme, the inactive, insoluble proteins were isolated and refolded as described:

Logarithmically growing 0.5 L cultures of JM109 cells bearing either pcWin2-MBP-mST6GalNAcI D32, E52, S127, S186, or pcWin2-MBP-hST6GalNAcI K36 were induced with 1 mM IPTG overnight at 37° C. Cells were collected by centrifugation, and lysed by mechanical disruption in a microfluidizer in 100 mL of 20 mM Tris pH8, 5 mM EDTA. Insoluble matter was collected by centrifugation at 7000×g for 20 minutes. The supernatants were discarded, and the pellets were washed with a high salt buffer (20 mM Tris pH 7.4, 1M NaCl, 5 mM EDTA), detergent buffer (25 mM Tris pH 8, 1% Na-deoxycholate, 1% Triton×100, 100 mM NaCl, 5 mM EDTA), and TE (10 n Tris pH 8, 1 mM EDTA). Each wash was in 100 mL, and the pellet was collected by centrifugation as described above. Following the washing, the inclusion body pellets were aliquoted and stored at −80° C.

To screen for conditions that allow proper refolding and thus recovery of ST6GalNAcI activity, aliquots of the mouse and human ST6GalNAcI fusion protein inclusion bodies were solubilized in 6M guanidine, 10 mM DTT, 1×TBS. Protein concentration was normalized by Bradford assay, and the solubilized proteins were transferred to a series of commercially available protein refolding buffers. Refolds were carried out in 0.25 mL at 0.2 mg/mL overnight at 4° C. in a 96-well plate with shaking. The refolds were transferred to a 96-well dialysis plate (25000 MWCO) and dialyzed against 1×TBS, 0.05% Tween-80 for four hours at 4° C., followed by overnight dialysis against 10 mM BisTris pH 7.1, 100 mM NaCl, 0.05% Tween-80 at 4° C.

Refolded recombinant ST6GalNAcI fusion proteins were tested for activity in a 384-well solid phase activity assay. Briefly, the activity assay detects the ST6GalNAcI-mediated transfer of a biotinylated sialic acid from biotinylated CMP-NAN to the surface of an asialo-bovine submaxillary mucin-coated well in a 384-well plate. Each reaction (13.5 μL refold+1.5 μL 10× reaction buffer) was performed in quadruplicate. 10× reaction buffer was 0.2M BisTris ph 6.7, 25 mM MgCl2, 25 mM MnCl2, 0.5% Tween-80, and 1 mM donor. After overnight incubation at 37° C., the plate was washed with excess 1×TBS, 0.05% Tween-20, and biotin detected with europium-labeled streptavidin as per manufacturer's instructions (Perkin Elmer). Europium fluorescence levels retained on the plate, indicative of ST6GalNAcI activity, were documented with a Perkin Elmer Victor3V plate reader, and expression and activity results are summarized in Table 21. Three of the refolded ST6GalNAcI fusion proteins had detectable activity.

TABLE 21
Summary of refolded ST6GalNAcI fusion proteins tested
for activity by solid phase assay.

| | Refolded protein | Refolded protein |
|---|---|---|
| | detected by SDS- | activity detected |
| Construct | PAGE | by solid phase assay |
| MBP-mST6GalNAcI D32 | + | − |
| MBP-mST6GalNAcI E52 | ++ | − |
| MBP-mST6GalNAcI S127 | +++ | + |
| MBP-mST6GalNAcI S186 | +/− | +/− |
| MBP-hST6GalNAcI K36 | +/− | + |

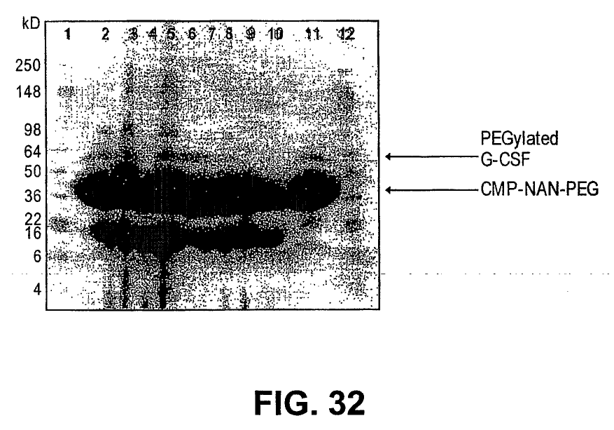

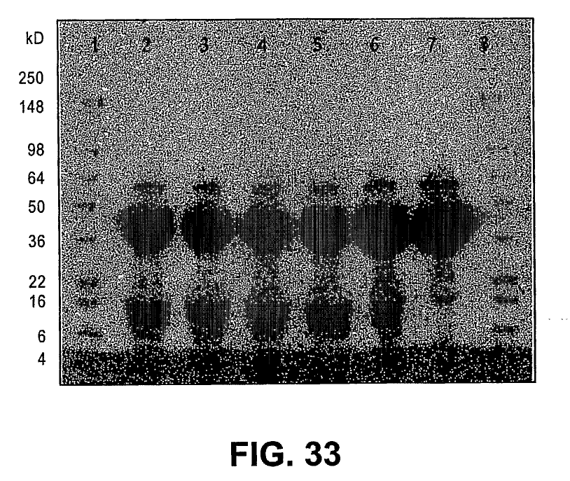

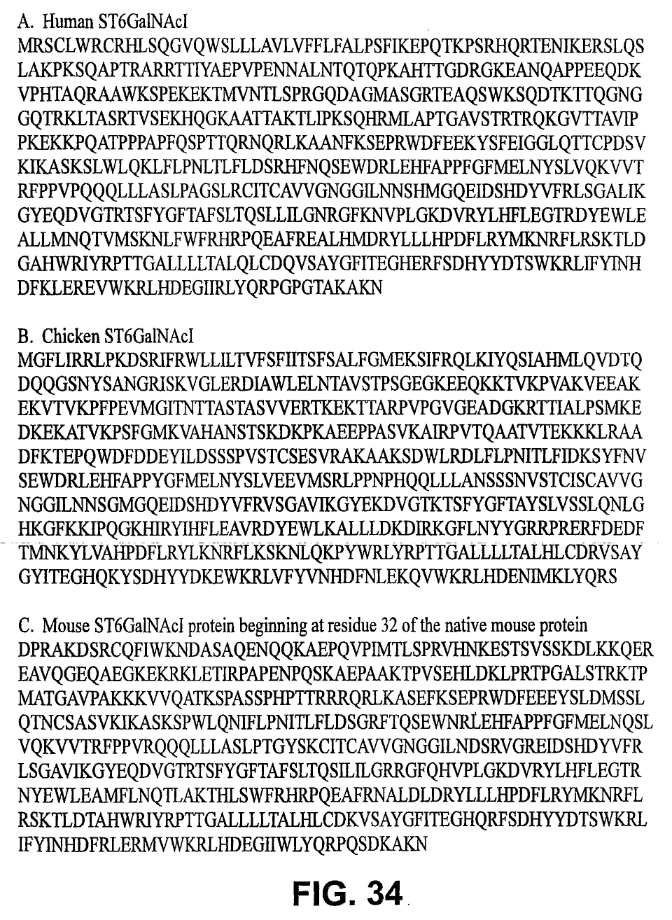

In summary, four N-terminal deletions of chicken ST6GalNAcI were successfully expressed in Sf9 cells as secreted, active enzymes. Maximal activity levels for the four active clones varied, with K232 VS4-001 at 19 U/L, Δ48 at 47 U/L, Δ152 at 44.1 U/L, and Δ225 at 27.9 U/L. Additionally, mutant chicken ST6GalNAcI produced in Δ48, Δ152 and Δ225 viral stocks were equally able to sialylPEGylate GalNAc−O-G-CSF (FIGS. 32 and 33).

### Example 7

### Generation of Additional Human ST6GalNAcI Proteins

Cloning hST6GalNAcI truncations: The following oligos: hST6-T73-hST6-G273 and hST6CooH were used to amplify various human ST6GalNAcI truncations

TABLE I
Truncation oligos for hST6GalNAcI.

| | |
|---|---|
| hST6-T73 | 5′TATTGGATCCACAACCATCTATGCAGAGCCAG |
| hST6-E110 | 5′TATTGGATCCGAGGAGCAGGACAAGGTGCCC |
| hST6-M134 | 5′TATTGGATCCATGGTGAACACACTGTCACCCA |
| hST6-T171 | 5′TATTGGATCCACCAGGAAGCTGACGGCCTCCA |
| hST6-A233 | 5′TATTGGATCCGCCACCCCACCCCCTGCCCCTT |
| hST6-G273 | 5′TATTGGATCCGGAGGCCTTCAGACGACTTGCC |
| hST6-CooH | 5′GCGCTCTAGATCAGTTCTTGGCTTTGGCAGTTCC |

The BamHI restriction site for oligos, hST6-T73 - G273 and XbaI restriction site for hST6-CooH oligo were underlined.

Template DNA: phST6GalNAcI K36 (the plasmid carrying Δ35 truncation of hST6GalNAcI gene)

PCR reactions: Fifty μl reactions were carried out using Herculase® Enhanced DNA polymerase (Stratagene) under PCR conditions: 30 cycles: 92° C., 45 s; 61° C., 1 min; 72° C., 3 min; and 4 cycles: 92° C., 45 s; 61° C., 1 min; 72° C., 10 min.

Agarose gel analysis: Three μl aliquots from the PCR reactions were analyzed in 1% agarose gel in TAE buffer stained with EtBr.

Cloning hST6GalNAcI truncations: The PCR amplified DNA fragments were purified using Millipore Ultrafree DA cartridges from the agarose gel and concentrated using Amicon microcon YM-100 filters. One to two ul aliquots from purified DNA fragments were used in Zero Blunt® TOPO® PCR cloning kit (Invitrogen). The reactions were transformed into competent TOP10 E. coli cells (Invitrogen). The following colonies obtained after 50 μl transformants were introduced onto Martone Agar Kan50 plates (Teknova)

| Truncation | # of colonies |
|---|---|
| K36 | 6 |
| T73 | 25 |
| E110 | 9 |
| M134 | 15 |
| T171 | 34 |
| A233 | 32 |
| G273 | 4 |

The plasmids DNAs were obtained from the cultures after growing the selected colonies (4-5 from each truncation) in 5 mls of Martone L-Broth liquid media (Teknova) supplemented with 50 μg/ml Kanamycin.

Screening hST6GalNAcI clones: The plasmid DNAs were purified from 4 ml overnight cultures using Wizards Plus SV Minipreps DNA purification system. The purified plasmids (10 μl) were digested with BamHI and XbaI restriction enzymes followed by agarose gel analysis [1.2% E-gel (Invitrogen)] to confirm the correct inserts (truncations).

The hST6GalNAcI truncations above were cloned into baculovirus expression vector, pAcGP67B, and expressed in SF9 insect cell culture. ST6GalNAcI activities were determined in the samples obtained from the infected cultures and results are shown in FIG. 38. Each of the truncated human ST6GalNAcI proteins had detectable activity after expression in the bacculoviral system. The hST6-E 10 protein had the highest activity.

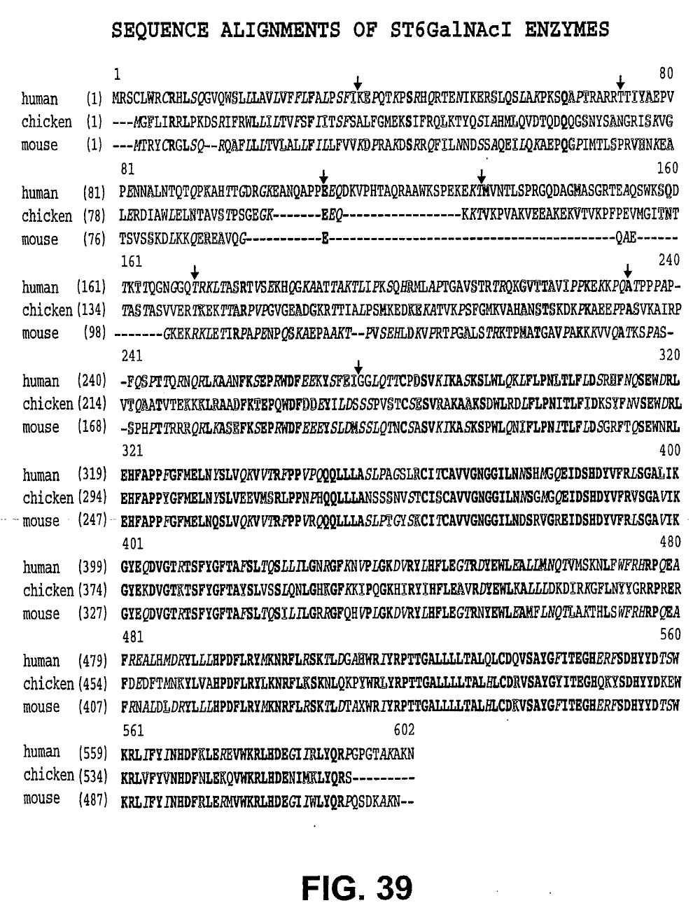

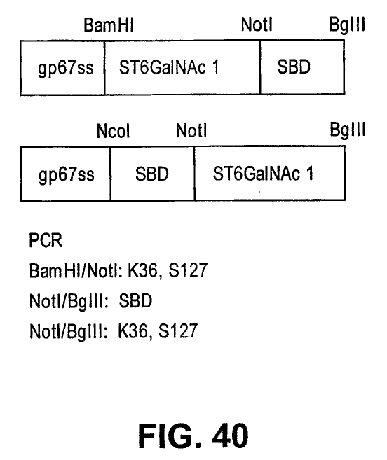

The hST6GalNAcI truncations are shown in FIG. 39. The figure also shows an alignment of the human sequence with the mouse and chicken proteins and identifies identical and conserved amino acid residues between the proteins.

### Example 8

### Truncated ST6GalNAcI Proteins that Comprise SBD Sequences

N-acetylgalactosamine-α-2,6-sialyltransferase (ST6GalNAc 1) transfers sialic acid from CMP-sialic acid, by an α-2,6 linkage, onto a N-acetylgalactosamine (GalNAc) residue, O-linked to a threonine or serine of a glycoprotein.

This report describes the cloning and expression of the SBD tag at the N-terminal and the C-terminal of the human (SBD-K36, K36-SBD) and mouse (SBD-S127, S127-SBD) ST6GalNAcI in Spodoptera frugiperda (Sf9) cells, using the pAcGP67 baculovirus expression system.

All four viral stocks were used to infect SF9 cells (150 mL scale) for 96 hours and the resultant supernatants were isolated on β-cyclodextrin column, concentrated and assayed for both sialylation of asialo bovine submaxillary mucin and sialylPEGylation of G-CSF.

### METHODS

### 1. Construct Design

A three way fusion among the gp67 secretion peptide, the ST6GalNAcI coding sequence and the SBD coding sequence was constructed. Based on the restriction maps (Appendix 4) of ST6GalNAcI and S B D and the multiple cloning sites in pAcGP67B vector, NcoI/NotI/BgtII was chosen for cloning the SBD-ST6GalNAcI constructs and BamHI/NotI/BglII was chosen for cloning ST6GalNAcI-SBD constructs. The NotI site introduced four amino acids (WRPP or RRPP) between the SBD and ST6GalNAcI coding sequences. This extension could help to separate these two protein domains. The SBD gene codon optimized for E. coli was not used in this work. The original A. niger SBD coding sequence was chosen, as it was determined that the codon codon bias of SF9 cells would be closer to that of the eukaryotic A. niger as opposed to the prokaryotic E. coli.

2. PCR Reactions

| | |
|---|---|
| 5 μl | 10x PCR Buffer |
| 2 μl | 10 mM dNTP |
| 1 μl | 5′ primer (10 pmol/μl) |
| 1 μl | 3′ primer (10 pmol/μl) |
| 2 μl | DMSO |
| 1 μl | DNA template (10 ng/μl) |

### 0.5 ul Herculase (Stratagene, Cat # 600260-51)

37.5 μl PCR grade H₂O

The PCR Program used for K36 and S127 was a) 95° C. 3 min; b) 95° C., 45 sec; 42° C. 45 sec, 72° C. 1.5 min for 5 cycles; c) 95° C., 45 sec; 54° C. 45 sec, 72° C. 1.5 min for 30 cycles; d) 72° C. 10 min; e) 4° C. pause. (LL774, pg 51). PCR were performed using a T3 Thermocycler.

The PCP Program used for SBD was a) 95° C. 3 min; b) 95° C., 45 sec; 40° C. 45 sec, 72° C. 1 min for 5 cycles; c) 95° C., 45 sec; 55° C. 45 sec, 72° C. 1 min for 30 cycles; d) 72° C. 10 min; e) 4° C. pause. (LL774, pg 51). PCR were performed using a T3 Thermocycler.

### 3. Gel Extraction

A MinElute Gel Extraction Kit was used to isolate all the PCR bands. The DNA was eluted in 20 μl 1×TE (10 mM Tris-HCl, 0.1 mM EDTA, pH 7.5).

4. pCRBlunt Ligation and Transformation
4.5 μl PCR product

### 1.0 μl Salt Solution

0.5 μl TOPO pCR4

The reaction was incubated at room temperature for 9 min.

Two microliters of each ligation mixture was added to separate eppendorf tubes containing 25 μl of Top 10 cells. The following incubations of each were performed: on ice 5 min, 42° C. 45 sec, on ice 2 min, adding 0.1 mL SOC then 37° C. 1 hour. After incubation, 120 μl of the mixture was spread on Kanamycin plate. About 7 to 70 colonies were generated for each transformation (LL774, pg 51).

### Plasmid DNA Minipreps

Single colonies were picked and grown, overnight at 37° C. in 2 mL terrific broth medium containing 50 μg/mL Kanamycin. The correct insert was checked with the pairwise restriction enzymes whose sites were designed into the PCR primers.

### 5. Subcloning

The pAcGP67B vector and each insert in the pCRBlunt vector were digested with the appropriate, pairwise restriction enzymes. The digested DNA was separated on 0.8% agarose gels. The corresponding bands were cut out with a surgical blade and DNA was extracted from the gel using the MiniElute Kit. The insert and vector were ligated together using T4 DNA ligase. The ligation mixture was transformed into Top10 cells and spread on ampicillin (carbenicillin) plates. After overnight incubation at 37° C., several colonies were picked and screened for the correct insert and vector for each plasmid

### 6. Cotransfection

In a 25 cm² falcon flask, 1×10⁶ Sf9 cells were seeded (50 to 70% confluence). Linearized BaculoGold DNA (0.5 μg) was mixed with 2 μg recombinant plasmid DNA and 100 μL of SF900 II SFM in an eppendorf. In another tube, 6 μL of cellfectin was mixed with 100 μL SF900 II SFM. The two mixtures were combined and incubated at room temperature for 15 to 45 min. The medium in the flask was removed and Sf9 cells were covered with the DNA mixture. An additional 0.8 mL of Sf900 μl SFM was added to the flask and incubated at 27° C. for 4 hours. After the incubation, the DNA mixture and cellfectin were removed and 3 mL of fresh Sf900 If SFM was added to the flask. The S19 cells in the flask were incubated, without shaking, for 3 days at 27° C. Visible infection could be seen after 72 hours (LL774, pg 89).

7. Recombinant Baculovirus amplification

Following the 3 day incubation, the culture supernatant was cleared by centrifugation at 1,000×g for 10 min. This supernatant was labeled the Passage Zero (P0) viral amplification stock.

P0 viral stock (0.5 mL) was incubated with a 50 mL suspension culture of Sf9 cells (1×10⁶ cells/mL). The incubation was done at 27° C., with stirring (100 rpm) for 3 days. The culture was harvested by centrifugation in a Corning sterile conical centrifuge tube at 5000 rpm (7,000×g) for 30 min at 4° C. and the resultant supernatant was labeled the Passage 1 (P1) viral amplification stock (LL774, pg 96).

Three mL of the P2 viral stock was incubated with a 150 mL suspension culture of Sf9 cells (1×10⁶ cells/mL). The incubation was done at 27° C., with stirring (100 rpm) for 66 hours. The supernatant, isolated as described for the P1 viral stock, was labeled the Passage 2 (P2) viral amplification stock (LL774, pg 96, 103).

P0 stored at −80° C. P1 and P2 were stored at 4° C. in the dark. The titer of the recombinant baculovirus at P2 was determined by plaque assay.

### 8. Low MOI Protocol

### LL774, pg 120

### Materials:

30 mL of Sf9 cells in 250 mL shake flask. Total flasks: 10.
The targeting cell concentration is: 1.5E6 cells/mL.
The targeting MOI is: 5E-4 to 5E-8.
Baculovirus virus:

| | |
|---|---|
| SBD-K36 | 2.55 × 10⁷ pfu/mL |
| K36-SBD | 2.25 × 10⁷ pfu/mL |

### Calculation:

Total cells: 1.5e6×30-45e6
Total virus for the highest MOI:
SBD-K36 (5E-4×45e6)/2.55E7=22500/2.55E7=0.88 μl
K36/SBD (5E-4×45e6)/2-25E7=22500/2.25E7=1.00 μl

### Dilution Procedures:

Dilute virus by:
8.8 μl SBD-K36 virus+1 mL Sf90011 SFM for MOI 5e-3
   10.0 μl K36-SBD virus+1 mL Sf 900II SFM for MOI 5e-3

### 0.2 mL 5E-3+1.8 mL Sf 900II SFM for MOI 5E-4

### 0.2 mL 5E-4+1.8 mL Sf 900II SFM for MOI 5E-5

### 0.2 mL 5E-5+1.8 mL Sf 900II SFM for MOI 5E-6

### 0.2 mL 5E-6+1.8 mL Sf 900 II SFM for MOI 5E-7

### 0.2 mL 5E-7+1.8 mL Sf 900 II SFM for MOI 5E-8

### Experiments:

Start experiments by adding 1 mL of each dilution to 30 mL of Sf 9 cells.
Check cell concentration and take 1 mL sample for radioactive assay on Day 4, Day 5, Day 6 and Day 7.
Summary results.

### Note:

The actual starting cell concentration is 1.47E6.
The cells were in PSG16.

### 9. Protein Production

Recombinant protein was produced by infecting 150 mL of 1.5×10⁶ cells/mL Sf9 cells with 75 μl of the P2 viral stock. The culture was incubated at 27° C., with stirring (100 rpm) for 96 hours. The supernatant was isolated as described for the P1 and P2 viral stocks. The MOI used for infection were: SBD-K36, 0.0085; K36-SBD, 0.0075; SBD-S127, 0.013; S127-SBD, 0.013 (LL774 pg 103, 120).

### 10. Purification of ST6GalNAcI Enzyme Using SBD Tag on β-Cyclodextrin Column

Human and mouse ST6GalNAcI fused with SBD tag was isolated from Sf9 cell supernatant by passage through a β-cyclodextrin column. Either 22.5 mU or 182.4 mU of SBD-human or mouse ST6GalNAcI, respectively, were loaded onto separate β-cyclodextrin columns (bed volume 7.5 mL) at about 0.4 mL/min at 4° C. The column was washed with 80 to 100 mL of Wash Buffer (1×PBS pH 7.4 Fisher Cat # BP-399-500). The bound enzyme was eluted from the column by Elution Buffer (3 mM β-cyclodextrin Sigma Cat # C4767 in Wash Buffer). Twelve fractions of 1 to 2 mL were collected. The elution profile was recorded as OD₂₈₀. The peak fractions were pooled and concentrated using a VIVASPIN 6 mL or 20 mL concentrator on Joann centrifuge at 4° C. for about 30 min at 7500 rpm (−6000 g). One mL of Wash Buffer was added to the concentrator at this point and continued to concentrate to a final volume of 100 to 200 μL. The concentrated product was tested for sialyation and sialylPEGylation.

The β-cycledextrin column was regenerated using 1 M NaCl in Wash buffer and then equilibrated with Wash Buffer or soaked in 0.5 M NaOH overnight. The column was next washed with H₂O until the pH reached 7.0 and then equilibrated with Wash Buffer.

### 11. Sialylation Radioactive Assay

Radioactive assays measured the transfer of ¹⁴C_Sialic acids from ¹⁴C-CMP-sialic acid to asialo-bovine submaxillary mucin (see DR-518-04 for details).

12. SialylPEGylation Assay
LL774, pg 163

| | |
|---|---|
| G-CSF-O-GalNAc (0.4 mg/mL) | 10.0 μL |
| 125 mM MnCl₂ | 0.5 μL |
| CMP-SA-PEG-20K | 0.5 μL |
| ST6GalNAcl | 2.0 μL |
| 100 mM Bis-Tris pH 6.5 | 10.0 μL |
| Total volume | 23.0 μL |

The reaction mixture was incubated, at 33° C., with no shaking for 66 hours.

After incubation, 2.5 μL of 5×SDS Sample Buffer (no DTT) was added to 5 μl of reaction with 5 μl of water and was loaded onto a 4 to 20% SDS-PAGE gradient gel without heating the samples

PEGylated G-CSF was detected by iodine staining of the gel.

To the rest reaction mixture, 42 μl of water was added and the sample was analyzed by HPLC.

GalNActylation reaction:

| | |
|---|---|
| G-CSF (~1 mg/mL in 40 mM Bis-Tris pH 6.5) | 140 μl |
| 100 mM MnCl₂ | 3 μl |
| 30 mM UDP-GalNAc | 9 μl |
| 100 mM Bis-Tris pH 6.5 | 113 μl |
| GalNAcT2 | 5 μl |

33° C. no shaking for 2 days.

### Results

### 1. PCR Results

The correct length PCR bands of K36 BamHI/NotI, S127BamHI/NotI, K36 NotI/BglII, S127 NotI/BglLL and SBD NotI/BglII were generated.

### 2. Cloning Results

The correct length clones were generated.

### 3. Titers of Recombinant (P2) Baculovirus Stocks of the ST6GalNAcI Clones

| | |
|---|---|
| SBD-K36 | 2.55 × 10⁷ |
| K36-SBD | 2.25 × 10⁷ |
| SBD-S127 | 3.85 × 10⁷ |
| S127-SBD | 3.95 × 10⁷ |

### 4. Summary of Sialylation Activity in Radioactive Assay

| | Average | Average |
|---|---|---|
| | Activity (Units/L) | Activity (Units/L) |
| Samples | Assay date Sep. 2, 2004 | Assay date Sep. 27, 2004 |
| SBD-K36 P2 | 0.512 | — |
| K36-SBD P2 | 0.279 | — |
| SBD-S127 P2 | 0.206 | — |
| S127-SBD P2 | 0.201 | — |
| SBD-K36 175 mL | — | 0.851 |
| K36-SBD 175 mL | — | 0.619 |
| SBD-S127 175 mL | — | 0.264 |
| S127-SBD 175 mL | — | 0.397 |
| SBD-K36 300 mL | — | 0.638 |
| K36-SBD 300 mL | — | 0.414 |

### 5. Ultra Low MOI Study Results

ST6GalNAc 1 Low MOI Study

| | | Activity in Unit/L | | | |
|---|---|---|---|---|---|
| | | Day | | | |
| MOI | | 4 | 5 | 6 | 7 |
| 5.00E−04 | SBD-K36 | 1.222 | 1.311 | 1.304 | 1.532 |
| 5.00E−05 | SBD-K36 | 1.934 | 1.786 | 1.779 | 1.751 |
| 5.00E−06 | SBD-K36 | 2.815 | 2.632 | 2.407 | 1.873 |
| 5.00E−07 | SBD-K36 | 0.900 | 1.831 | 3.087 | 3.253 |
| 5.00E−08 | SBD-K36 | 0.080 | 0.247 | 0.920 | 1.256 |
| 5.00E−04 | K36-SBD | 0.638 | 0.559 | 0.796 | 0.630 |
| 5.00E−05 | K36-SBD | 0.923 | 0.673 | 1.387 | 0.696 |
| 5.00E−06 | K36-SBD | 0.695 | 0.945 | 1.012 | 0.956 |
| 5.00E−07 | K36-SBD | 0.901 | 1.456 | 2.264 | 1.798 |
| 5.00E−08 | K36-SBD | 0.136 | 0.175 | 0.494 | 0.439 |
| | | Sf 9 (E6 cells/mL) | | | |
| | | Day | | | |
| MOI | | 4 | 5 | 6 | 7 |
| 5.00E−04 | SBD-K36 | 3.65 | 1.85 | 2.30 | 1.00 |
| 5.00E−05 | SBD-K36 | 6.25 | 3.60 | 3.10 | 1.75 |
| 5.00E−06 | SBD-K36 | 9.10 | 5.10 | 2.90 | 1.85 |
| 5.00E−07 | SBD-K36 | 11.40 | 3.05 | 8.80 | 6.85 |
| 5.00E−08 | SBD-K36 | 11.60 | 12.00 | 2.60 | 7.70 |
| 5.00E−04 | K36-SBD | 3.75 | 1.40 | 1.70 | 0.45 |
| 5.00E−05 | K36-SBD | 4.60 | 2.40 | 2.50 | 1.15 |
| 5.00E−06 | K36-SBD | 8.90 | 4.50 | 4.10 | 2.15 |
| 5.00E−07 | K36-SBD | 9.90 | 8.50 | 11.10 | 6.75 |
| 5.00E−08 | K36-SBD | 11.90 | 11.00 | 2.30 | 5.85 |

Starting cell concentration was 1.47E6

cells/mL, LL-774 pg 120

### 6. Summary of the Purification of Human and Mouse ST6GalNAc 1 SBD Fusion Proteins on β-Cyclodextrin Column.

| | Species | | | |
|---|---|---|---|---|
| | SBD-K36 | K36-SBD | SBD-S127 | S127-SBD |
| | Date | | | |
| | Sep. 14, | | | Sep. 17, |
| | 2004 | Sep. 15, 2004 | Sep. 16, 2004 | 2004 |
| Load (mu/mL) | 1.459 | 0.826 | 0.167 | 0.297 |
| Load (mL) | 125 | 125 | 135 | 138 |
| Load (mu) | 182.375 | 103.25 | 22.545 | 40.986 |
| FT (mu/mL) | 0.452 | 0.015 | 0.016 | 0.005 |
| FT (mL) | 125 | 125 | 135 | 138 |
| FT (mu) | 52.5 | 1.875 | 2.16 | 0.69 |
| Wash (mu/mL) | 0.091 | 0.029 | 0.033 | −0.028 |
| Wash (mL) | 80 | 80 | 100 | 105 |
| Wash (mu) | 7.28 | 2.32 | 3.3 | 0 |
| Bound (%) | 67.22% | 95.94% | 75.78% | 98.32% |
| Bound (mu) | 122.58 | 99.055 | 17.085 | 40.296 |
| Elute (mu/mL) | 122.908 | 22.928 | 5.510 | 15.943 |
| Elute (mL) | 0.26 | 0.42 | 0.46 | 0.35 |
| Elute (mu) | 31.956 | 9.630 | 2.535 | 5.58 |
| Recovered | 26.1% | 9.7% | 14.8% | 13.8% |
| PEG-gCSF | 72.9% | 30.7% | 11.0% | 9.3% |

Two human ST6GalNAcI fusion constructs (SBD-K36 and K36-SBD) and two mouse ST6GalNAcI fusion constructs (SBD-127 and 127-SBD) have been successfully expressed in Sf9 cells as secreted, active enzymes and purified on a β-cyclodextrin column using their SBD tags.

The activity levels of purified, concentrated samples of the four active clones were:

| | |
|---|---|
| SBD-K36 | 122.9 U/L |
| K36-SBD | 22.9 U/L |
| SBD-S127 | 5.5 U/L |
| S127-SBD | 15.9 U/L |

All four enzymes were able to sialylPEGylate G-CSF.

Using ultra low MOIs, the activity expression of human ST6GalNAcI-SBD proteins was increased, with an MOI of 5e-7 with SBD-6 on Day 6 and Day 7 giving activity of 3-09 U/L and 3.25 U/L respectively and with an MOI of 5e-7 with K36-SBD on Day 6 giving activity was 2.26 U/L.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated truncated ST6GalNAcI polypeptide, wherein said truncated ST6GalNAcI polypeptide is lacking all or a portion of the ST6GalNAcI signal domain, and wherein said ST6GalNAcI polypeptide is selected from the group consisting of a human ST6GalNAcI polypeptide and a chicken ST6GalNAcI polypeptide, with the proviso that said polypeptide is not a chicken ST6GalNAcI polypeptide truncation mutant lacking amino acid residues 1-232.

2. The isolated truncated ST6GalNAcI polypeptide of claim 1, wherein said truncated ST6GalNAcI polypeptide is further lacking all or a portion of the ST6GalNAcI transmembrane domain.

3. The isolated truncated ST6GalNAcI polypeptide of claim 2, wherein said truncated ST6GalNAcI polypeptide is further lacking all or a portion of the ST6GalNAcI stem domain.

4. The isolated truncated ST6GalNAcI polypeptide of claim 1, wherein said truncated ST6GalNAcI polypeptide has at least 90% identity with a polypeptide selected from the group consisting of SEQ ID NO:10, SEQ ID NO:12, SEQ ID NO:14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, and Δ225 of the chicken sequence shown in FIG. 31.

5. The isolated truncated ST6GalNAcI polypeptide of claim 1, wherein said truncated ST6GalNAcI polypeptide comprises an amino acid sequence selected from the group consisting of SEQ ID NO: 10, SEQ ID NO: 12, SEQ ID NO: 14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ID NO:28, SEQ ID NO:30, SEQ ID NO:32, and Δ225 of the chicken sequence shown in FIG. 31.

6. The isolated truncated ST6GalNAcI polypeptide of claim 1, wherein said truncated ST6GalNAcI polypeptide consists of an amino acid sequence selected from the group consisting of SEQ ID NO:10, SEQ ID NO: 12, SEQ ID NO: 14, Δ35 of the human sequence shown in FIG. 31, Δ72 of the human sequence shown in FIG. 31, Δ109 of the human sequence shown in FIG. 31, Δ133 of the human sequence shown in FIG. 31, Δ170 of the human sequence shown in FIG. 31, Δ232 of the human sequence shown in FIG. 31, Δ272 of the human sequence shown in FIG. 31, SEQ ED NO:28, SEQ ID NO:30, SEQ ID NO:32, and Δ225 of the chicken sequence shown in FIG. 31.

7. An isolated chimeric polypeptide comprising a tag polypeptide covalently linked to the isolated truncated ST6GalNAcI polypeptide of claim 1.

8. The isolated chimeric polypeptide of claim 7, wherein said tag polypeptide is selected from the group consisting of a maltose binding protein, a histidine tag, a Factor IX tag, a glutathione-S-transferase tag, a FLAG-tag, and a starch binding domain tag.

9. An isolated nucleic acid that comprises and nucleic acid sequence that encodes isolated truncated ST6GalNAcI polypeptide of claim 1 or claim 4.

10. The isolated nucleic acid of claim 9, said nucleic acid further comprising a promoter/regulatory sequence operably linked thereto.

11. An expression vector comprising the isolated nucleic acid of claim 9.

12. A recombinant host cell comprising the isolated nucleic acid of claim 11.

13. A recombinant cell of claim 12, wherein said recombinant cell is a eukaryotic cell or a prokaryotic cell.

14. The recombinant cell of claim 13, wherein said eukaryotic cell is selected from the group consisting of a mammalian cell, an insect cell and a fungal cell.

15. The recombinant cell of claim 14, wherein said insect cell is selected from the group consisting of an SF9 cell, an SF9+ cell, an Sf21 cell, a HIGH FIVE cell or Drosophila Schneider S2 cell.

16. The recombinant cell of claim 13, wherein said prokaryotic cell is selected from the group consisting of an E. coli cell and a B. subtilis cell.

17. A method of producing a truncated ST6GalNAcI polypeptide, the method comprising growing the recombinant cell of claim 13 under conditions suitable for expression of the truncated ST6GalNAcI polypeptide.

18. A method of catalyzing the transfer of a sialic acid moiety to an acceptor moiety comprising incubating the polypeptide of claim 1 with a sialic acid moiety and an acceptor moiety, wherein said polypeptide mediates the covalent linkage of said sialic acid moiety to said acceptor moiety, thereby catalyzing the transfer of a sialic acid moiety to an acceptor moiety.

19. A method of catalyzing the transfer of a sialic acid moiety to an acceptor moiety comprising incubating the polypeptide of claim 1 with a cytidinemonophosphate-sialic acid (CMP-NAN) sialic acid donor and an asialo bovine submaxillary mucin acceptor moiety, wherein said polypeptide mediates the transfer of said sialic acid moiety from said CMP-NAN sialic acid donor to said asialo bovine submaxillary mucin acceptor, thereby catalyzing the transfer of a sialic acid moiety to an acceptor moiety.

20. A method of catalyzing the transfer of a sialic acid moiety to an acceptor moiety comprising incubating the polypeptide of claim 1 with a cytidinemonophosphate-sialic acid (CMP-NAN) sialic acid donor and a polypeptide acceptor, wherein said polypeptide acceptor is selected from the group consisting of erythropoietin, human growth hormone, granulocyte colony stimulating factor, interferons alpha, -beta, and -gamma, Factor IX, follicle stimulating hormone, interleukin-2, erythropoietin, anti-TNF-alpha, and a lysosomal hydrolase.

21. The method of claim 20, wherein said polypeptide acceptor is a glycopeptide.

22. The method of claim 19 or claim 20, further wherein said sialic acid moiety comprises a polyethylene glycol moiety.

23. The method of claim 19 or claim 20, wherein said method is carried out on a commercial scale.


SEQUENCE LISTING (119 sequences)

SEQ ID NO: 1 - DNA, 1800 bp, Homo sapiens
  wild-type N-acetylgalactosamine-alpha2,6-sialyltransferase I (ST6GalNAcI)
atgaggtcctgcctgtggagatgcaggcacctgagccaaggcgtccagtggtccttgcttctggctgtcctggtcttctttctcttcgccttgccctcttttattaaggagcctcaaacaaagccttccaggcatcaacgcacagagaacattaaagaaaggtctctacagtccctggcaaagcctaagtcccaggcacccacaagggcaaggaggacaaccatctatgcagagccagtgccagagaacaatgccctcaacacacaaacccagcccaaggcccacaccaccggagacagaggaaaggaggccaaccaggcaccgccggaggagcaggacaaggtgccccacacagcacagagggcagcatggaagagcccagaaaaagagaaaaccatggtgaacacactgtcacccagagggcaagatgcagggatggcctctggcaggacagaggcacaatcatggaagagccaggacacaaagacgaccaaaggaaatgggggccagaccaggaagctgacggcctccaggacggtgtcagagaagcaccagggcaaagcggcaaccacagccaagacgctcattcccaaaagtcagcacagaatgctggctcccacaggagcagtgtcaacaaggacgagacagaaaggagtgaccacagcagtcatcccacctaaggagaagaaacctcaggccaccccaccccctgcccctttccagagccccacgacgcagagaaaccaaagactgaaggccgccaacttcaaatctgagcctcggtgggattttgaggaaaaatacagcttcgaaataggaggccttcagacgacttgccctgactctgtgaagatcaaagcctccaagtcgctgtggctccagaaactctttctgcccaacctcactctcttcctggactccagacacttcaaccagagtgagtgggaccgcctggaacactttgcaccaccctttggcttcatggagctcaactactccttggtgcagaaggtcgtgacacgcttccctccagtgccccagcagcagctgctcctggccagcctccccgctgggagcctccggtgcatcacctgtgccgtggtgggcaacgggggcatcctgaacaactcccacataggccaggagatagacagtcacgactacgtgttccgattgagcggagctctcattaaaggctacgaacaggatgtggggactcggacatccttctacggctttaccgccttctccctgacccagtcactccttatattgggcaatcggggtttcaagaacgtgcctcttgggaaggacgtccgctacttgcacttcctggaaggcacccgggactatgagtggctggaagcactgcttatgaatcagacggtgatgtcaaaaaaccttttctggttcaggcacagaccccaggaagcttttcgggaagccctgcacatggacaggtacctgttgctgcacccagactttctccgatacatgaagaacaggtttctgaggtctaagaccctggatggtgcccactggaggatataccgccccaccactggggcccttctgctgctcactgcccttcagctctgtgaccaggtgagtgcttatggcttcatcactgagggccatgagcgcttttctgatcactactatgatacatcatggaagcggctgatcttttacataaaccatgacttcaagctggagagagaagtctggaagcggctacacgatgaagggataatccggctgtaccagcgtcctggtcccggaactgccaaagccaagaac

SEQ ID NO: 2 - protein, 600 aa, Homo sapiens
  wild-type N-acetylgalactosamine-alpha2,6-sialyltransferase I (ST6GalNAcI), human ST6GalNAcI from pcDNA3.1(+)-hST6GalNAcI-N1C1#1
MRSCLWRCRHLSQGVQWSLLLAVLVFFLFALPSFIKEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTKGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHIGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 3 - DNA, 1641 bp, Mus sp.
  wild-type N-acetylgalactosamine-alpha2,6-sialyltransferase I (ST6GalNAcI)
atgctactagtaaatcagtcacaccaaggcttcaataaggaacacacaagcaagatggtaagcgctattgttttatatgtgcttttggcggcggcggcgcattctgcctttgcggcggatccaagggcaaaagattccaggtgccagttcatatggaagaatgacgcaagtgcccaggagaatcaacaaaaggctgagccccaagtacccatcatgacactgtcacccagagttcacaacaaggagtcaacctctgtcagttcaaaggacctgaagaaacaggagagagaggcagtccaaggagagcaagctgaggggaaggagaagaggaagttagagaccataaggccagcaccagagaatcctcagagcaaggcagagcctgctgcaaagacgcctgtgtcagaacacctggacaaactacccagaactccaggagcactgtcaacaaggaagacaccaatggccacaggagctgtcccagctaagaagaaagtggtccaggctaccaaatcccctgcctcttccccacaccccaccacacgaagaaggcaaaggctgaaggcctctgagttcaagtctgagcctcggtgggattttgaggaggaatatagcttggatatgagcagcctgcagacgaactgctctgcttctgtgaagatcaaggcctccaagtcaccatggctacagaatatctttctgcccaacatcactctgttcctggactctggacgcttcacccagagcgagtggaaccgtctagagcacttcgctcctccctttggcttcatggaactcaatcagtccctggtacagaaggtggtgacccgcttccctccagttcgccagcagcagctcctcctggccagcctccctactgggtactccaagtgtatcacctgtgctgtagtgggcaacgggggcatcctgaatgattcacgtgttggccgggagatagacagccatgactatgttttccgactgagtggagccgttattaaaggatatgaacaggatgtggggacccggacatccttctatggcttcactgctttctctctgacccagtctatcctcattttgggtagacgaggcttccagcatgtgcctctgggaaaggacgtccgatatctacacttcctggaaggcacccgggactatgagtggctggaagctatgtttttgaatcagaccttggcaaaaacccacctttcctggttcaggcataggcctcaggaagccttccggaatgccttggacttggatcgatacttgctgctgcacccagactttctccgttacatgaagaacaggtttctgaggtcaaagaccctggacactgcccattggagaatataccgccccaccactggtgccctcctgctgctcacagcccttcatctctgtgacaaggtcagcgcctatggcttcatcaccgagggccaccagcgcttctctgaccactactatgatacatcatggaaacggctcatcttttatatcaaccatgacttcaggttagagagaatggtgtggaagcggttgcatgatgaaggcatcatctggctctaccagcgtccacaaagtgacaaagcaaagaacggcggccgcgaacaaaaactcatctcagaagaggatctg

SEQ ID NO: 4 - protein, 547 aa, Mus sp.
  wild-type N-acetylgalactosamine-alpha2,6-sialyltransferase I (ST6GalNAcI), mouse ST6GalNAcI from pTS103
MLLVNQSHQGFNKEHTSKMVSAIVLYVLLAAAAHSAFAADPRAKDSRCQFIWKNDASAQENQQKAEPQVPIMTLSPRVHNKESTSVSSKDLKKQEREAVQGEQAEGKEKRKLETIRPAPENPQSKAEPAAKTPVSEHLDKLPRTPGALSTRKTPMATGAVPAKKKVVQATKSPASSPHPTTRRRQRLKASEFKSEPRWDFEEEYSLDMSSLQTNCSASVKIKASKSPWLQNIFLPNITLFLDSGRFTQSEWNRLEHFAPPFGFMELNQSLVQKVVTRFPPVRQQQLLLASLPTGYSKCITCAVVGNGGILNDSRVGREIDSHDYVFRLSGAVIKGYEQDVGTRTSFYGFTAFSLTQSILILGRRGFQHVPLGKDVRYLHFLEGTRDYEWLEAMFLNQTLAKTHLSWFRHRPQEAFRNALDLDRYLLLHPDFLRYMKNRFLRSKTLDTAHWRIYRPTTGALLLLTALHLCDKVSAYGFITEGHQRFSDHYYDTSWKRLIFYINHDFRLERMVWKRLHDEGIIWLYQRPQSDKAKNGGREQKLISEEDL

SEQ ID NO: 5 - DNA, 1698 bp, Gallus gallus
  wild-type N-acetylgalactosamine-alpha2,6-sialyltransferase I (ST6GalNAcI)
atggggtttttaatcagaaggcttcctaaagattccagaatattccgttggctccttattttaacagtcttttccttcatcattactagttttagcgccttgtttggcatggagaaaagcattttcaggcagctcaagatttaccaaagcattgcacatatgctacaagtggacacccaagatcagcaaggttcaaactattctgctaatgggagaatttcaaaggttggtttggagagagacattgcatggctcgaactgaatactgctgtgagtacaccaagtggggaagggaaggaagagcagaagaaaacagtgaaaccagttgccaaggtggaagaagccaaggagaaagtgactgtgaaaccattccctgaggtgatggggatcacaaatacaacagcatcaacagcctctgtggtggagagaacaaaggagaaaacaacagcgagaccagttccaggggtgggggaagctgatgggaagagaacaacgatagcacttcccagcatgaaggaagacaaagagaaggcgactgtgaaaccatcctttgggatgaaggtagctcatgcaaacagcacatccaaagataaaccaaaggcagaagagcctcctgcatcagtgaaagccataagacctgtgactcaggctgccacagtgacagagaagaagaaactgagggctgctgacttcaagactgagccacagtgggattttgatgatgagtacatactggatagctcatctccagtatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcc

SEQ ID NO: 6 - protein, 566 aa, Gallus gallus
  wild-type N-acetylgalactosamine-alpha2,6-sialyltransferase I (ST6GalNAcI)
MGFLIRRLPKDSRIFRWLLILTVFSFIITSFSALFGMEKSIFRQLKIYQSIAHMLQVDTQDQQGSNYSANGRISKVGLERDIAWLELNTAVSTPSGEGKEEQKKTVKPVAKVEEAKEKVTVKPFPEVMGITNTTASTASVVERTKEKTTARPVPGVGEADGKRTTIALPSMKEDKEKATVKPSFGMKVAHANSTSKDKPKAEEPPASVKAIRPVTQAATVTEKKKLRAADFKTEPQWDFDDEYILDSSSPVSTCSESVRAKAAKSDWLRDLFLPNITLFIDKSYFNVSEWDRLEHFAPPYGFMELNYSLVEEVMSRLPPNPHQQLLLANSSSNVSTCISCAVVGNGGILNNSGMGQEIDSHDYVFRVSGAVIKGYEKDVGTKTSFYGFTAYSLVSSLQNLGHKGFKKIPQGKHIRYIHFLEAVRDYEWLKALLLDKDIRKGFLNYYGRRPRERFDEDFTMNKYLVAHPDFLRYLKNRFLKSKNLQKPYWRLYRPTTGALLLLTALHLCDRVSAYGYITEGHQKYSDHYYDKEWKRLVFYVNHDFNLEKQVWKRLHDENIMKLYQRS

SEQ ID NO: 7 - DNA, 34 bp, Artificial Sequence
  Description of Artificial Sequence:PCR primer ch233XhoI for isolation of chicken ST6GalNAcI, PCR primer to generate delta232 mutant
gatcgcctcgagtcaggatctctggtagagcttc

SEQ ID NO: 8 - protein, 24 aa, Artificial Sequence
  Description of Artificial Sequence:N-terminal sequence of purified chicken ST6GalNAcI
VSTEDPKTEPQWDFDDEYILDSSS

SEQ ID NO: 9 - DNA, 1695 bp, Artificial Sequence
  Description of Artificial Sequence:delta35 human ST6GalNAcI
aaggagcctcaaacaaagccttccaggcatcaacgcacagagaacattaaagaaaggtctctacagtccctggcaaagcctaagtcccaggcacccacaagggcaaggaggacaaccatctatgcagagccagtgccagagaacaatgccctcaacacacaaacccagcccaaggcccacaccaccggagacagaggaaaggaggccaaccaggcaccgccggaggagcaggacaaggtgccccacacagcacagagggcagcatggaagagcccagaaaaagagaaaaccatggtgaacacactgtcacccagagggcaagatgcagggatggcctctggcaggacagaggcacaatcatggaagagccaggacacaaagacgaccaaaggaaatgggggccagaccaggaagctgacggcctccaggacggtgtcagagaagcaccagggcaaagcggcaaccacagccaagacgctcattcccaaaagtcagcacagaatgctggctcccacaggagcagtgtcaacaaggacgagacagaaaggagtgaccacagcagtcatcccacctaaggagaagaaacctcaggccaccccaccccctgcccctttccagagccccacgacgcagagaaaccaaagactgaaggccgccaacttcaaatctgagcctcggtgggattttgaggaaaaatacagcttcgaaataggaggccttcagacgacttgccctgactctgtgaagatcaaagcctccaagtcgctgtggctccagaaactctttctgcccaacctcactctcttcctggactccagacacttcaaccagagtgagtgggaccgcctggaacactttgcaccaccctttggcttcatggagctcaactactccttggtgcagaaggtcgtgacacgcttccctccagtgccccagcagcagctgctcctggccagcctccccgctgggagcctccggtgcatcacctgtgccgtggtgggcaacgggggcatcctgaacaactcccacataggccaggagatagacagtcacgactacgtgttccgattgagcggagctctcattaaaggctacgaacaggatgtggggactcggacatccttctacggctttaccgccttctccctgacccagtcactccttatattgggcaatcggggtttcaagaacgtgcctcttgggaaggacgtccgctacttgcacttcctggaaggcacccgggactatgagtggctggaagcactgcttatgaatcagacggtgatgtcaaaaaaccttttctggttcaggcacagaccccaggaagcttttcgggaagccctgcacatggacaggtacctgttgctgcacccagactttctccgatacatgaagaacaggtttctgaggtctaagaccctggatggtgcccactggaggatataccgccccaccactggggcccttctgctgctcactgcccttcagctctgtgaccaggtgagtgcttatggcttcatcactgagggccatgagcgcttttctgatcactactatgatacatcatggaagcggctgatcttttacataaaccatgacttcaagctggagagagaagtctggaagcggctacacgatgaagggataatccggctgtaccagcgtcctggtcccggaactgccaaagccaagaac

SEQ ID NO: 10 - protein, 565 aa, Artificial Sequence
  Description of Artificial Sequence:delta35 human ST6GalNAcI
KEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTKGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHIGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 11 - DNA, 1428 bp, Artificial Sequence
  Description of Artificial Sequence:delta124 human ST6GalNAcI
aagagcccagaaaaagagaaaaccatggtgaacacactgtcacccagagggcaagatgcagggatggcctctggcaggacagaggcacaatcatggaagagccaggacacaaagacgaccaaaggaaatgggggccagaccaggaagctgacggcctccaggacggtgtcagagaagcaccagggcaaagcggcaaccacagccaagacgctcattcccaaaagtcagcacagaatgctggctcccacaggagcagtgtcaacaaggacgagacagaaaggagtgaccacagcagtcatcccacctaaggagaagaaacctcaggccaccccaccccctgcccctttccagagccccacgacgcagagaaaccaaagactgaaggccgccaacttcaaatctgagcctcggtgggattttgaggaaaaatacagcttcgaaataggaggccttcagacgacttgccctgactctgtgaagatcaaagcctccaagtcgctgtggctccagaaactctttctgcccaacctcactctcttcctggactccagacacttcaaccagagtgagtgggaccgcctggaacactttgcaccaccctttggcttcatggagctcaactactccttggtgcagaaggtcgtgacacgcttccctccagtgccccagcagcagctgctcctggccagcctccccgctgggagcctccggtgcatcacctgtgccgtggtgggcaacgggggcatcctgaacaactcccacataggccaggagatagacagtcacgactacgtgttccgattgagcggagctctcattaaaggctacgaacaggatgtggggactcggacatccttctacggctttaccgccttctccctgacccagtcactccttatattgggcaatcggggtttcaagaacgtgcctcttgggaaggacgtccgctacttgcacttcctggaaggcacccgggactatgagtggctggaagcactgcttatgaatcagacggtgatgtcaaaaaaccttttctggttcaggcacagaccccaggaagcttttcgggaagccctgcacatggacaggtacctgttgctgcacccagactttctccgatacatgaagaacaggtttctgaggtctaagaccctggatggtgcccactggaggatataccgccccaccactggggcccttctgctgctcactgcccttcagctctgtgaccaggtgagtgcttatggcttcatcactgagggccatgagcgcttttctgatcactactatgatacatcatggaagcggctgatcttttacataaaccatgacttcaagctggagagagaagtctggaagcggctacacgatgaagggataatccggctgtaccagcgtcctggtcccggaactgccaaagccaagaac

SEQ ID NO: 12 - protein, 476 aa, Artificial Sequence
  Description of Artificial Sequence:delta124 human ST6GalNAcI
KSPEKEKTMVNTLSPRGQDAGMASGRTEAQSWKSQDTKTTKGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHIGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 13 - DNA, 1029 bp, Artificial Sequence
  Description of Artificial Sequence:delta257 human ST6GalNAcI
tctgagcctcggtgggattttgaggaaaaatacagcttcgaaataggaggccttcagacgacttgccctgactctgtgaagatcaaagcctccaagtcgctgtggctccagaaactctttctgcccaacctcactctcttcctggactccagacacttcaaccagagtgagtgggaccgcctggaacactttgcaccaccctttggcttcatggagctcaactactccttggtgcagaaggtcgtgacacgcttccctccagtgccccagcagcagctgctcctggccagcctccccgctgggagcctccggtgcatcacctgtgccgtggtgggcaacgggggcatcctgaacaactcccacataggccaggagatagacagtcacgactacgtgttccgattgagcggagctctcattaaaggctacgaacaggatgtggggactcggacatccttctacggctttaccgccttctccctgacccagtcactccttatattgggcaatcggggtttcaagaacgtgcctcttgggaaggacgtccgctacttgcacttcctggaaggcacccgggactatgagtggctggaagcactgcttatgaatcagacggtgatgtcaaaaaaccttttctggttcaggcacagaccccaggaagcttttcgggaagccctgcacatggacaggtacctgttgctgcacccagactttctccgatacatgaagaacaggtttctgaggtctaagaccctggatggtgcccactggaggatataccgccccaccactggggcccttctgctgctcactgcccttcagctctgtgaccaggtgagtgcttatggcttcatcactgagggccatgagcgcttttctgatcactactatgatacatcatggaagcggctgatcttttacataaaccatgacttcaagctggagagagaagtctggaagcggctacacgatgaagggataatccggctgtaccagcgtcctggtcccggaactgccaaagccaagaac

SEQ ID NO: 14 - protein, 343 aa, Artificial Sequence
  Description of Artificial Sequence:delta257 human ST6GalNAcI
SEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHIGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 15 - DNA, 1938 bp, Artificial Sequence
  Description of Artificial Sequence:pcDNA3.1(+)-hST6GalNAcI-N1C1#1
gatccactagtaacggccgccagtgtgctgaattcaggacatgcagaaccttcctctagaacccgacccaccaccatgaggtcctgcctgtggagatgcaggcacctgagccaaggcgtccagtggtccttgcttctggctgtcctggtcttctttctcttcgccttgccctcttttattaaggagcctcaaacaaagccttccaggcatcaacgcacagagaacattaaagaaaggtctctacagtccctggcaaagcctaagtcccaggcacccacaagggcaaggaggacaaccatctatgcagagccagtgccagagaacaatgccctcaacacacaaacccagcccaaggcccacaccaccggagacagaggaaaggaggccaaccaggcaccgccggaggagcaggacaaggtgccccacacagcacagagggcagcatggaagagcccagaaaaagagaaaaccatggtgaacacactgtcacccagagggcaagatgcagggatggcctctggcaggacagaggcacaatcatggaagagccaggacacaaagacgaccaaaggaaatgggggccagaccaggaagctgacggcctccaggacggtgtcagagaagcaccagggcaaagcggcaaccacagccaagacgctcattcccaaaagtcagcacagaatgctggctcccacaggagcagtgtcaacaaggacgagacagaaaggagtgaccacagcagtcatcccacctaaggagaagaaacctcaggccaccccaccccctgcccctttccagagccccacgacgcagagaaaccaaagactgaaggccgccaacttcaaatctgagcctcggtgggattttgaggaaaaatacagcttcgaaataggaggccttcagacgacttgccctgactctgtgaagatcaaagcctccaagtcgctgtggctccagaaactctttctgcccaacctcactctcttcctggactccagacacttcaaccagagtgagtgggaccgcctggaacactttgcaccaccctttggcttcatggagctcaactactccttggtgcagaaggtcgtgacacgcttccctccagtgccccagcagcagctgctcctggccagcctccccgctgggagcctccggtgcatcacctgtgccgtggtgggcaacgggggcatcctgaacaactcccacataggccaggagatagacagtcacgactacgtgttccgattgagcggagctctcattaaaggctacgaacaggatgtggggactcggacatccttctacggctttaccgccttctccctgacccagtcactccttatattgggcaatcggggtttcaagaacgtgcctcttgggaaggacgtccgctacttgcacttcctggaaggcacccgggactatgagtggctggaagcactgcttatgaatcagacggtgatgtcaaaaaaccttttctggttcaggcacagaccccaggaagcttttcgggaagccctgcacatggacaggtacctgttgctgcacccagactttctccgatacatgaagaacaggtttctgaggtctaagaccctggatggtgcccactggaggatataccgccccaccactggggcccttctgctgctcactgcccttcagctctgtgaccaggtgagtgcttatggcttcatcactgagggccatgagcgcttttctgatcactactatgatacatcatggaagcggctgatcttttacataaaccatgacttcaagctggagagagaagtctggaagcggctacacgatgaagggataatccggctgtaccagcgtcctggtcccggaactgccaaagccaagaactgaccggaattctgcagatatccagcacagtggcgaccgctcgagtctagagggcccgttaaa

SEQ ID NO: 16 - skipped

SEQ ID NO: 17 - DNA, 1485 bp, Artificial Sequence
  Description of Artificial Sequence:mouse N-acetylgalactosamine-alpha2,6-sialyltransferase I (ST6GalNAcI) beginning at residue 32 of the native mouse protein, mouse delta31
gatccaagggcaaaagattccaggtgccagttcatatggaagaatgacgcaagtgcccaggagaatcaacaaaaggctgagccccaagtacccatcatgacactgtcacccagagttcacaacaaggagtcaacctctgtcagttcaaaggacctgaagaaacaggagagagaggcagtccaaggagagcaagctgaggggaaggagaagaggaagttagagaccataaggccagcaccagagaatcctcagagcaaggcagagcctgctgcaaagacgcctgtgtcagaacacctggacaaactacccagaactccaggagcactgtcaacaaggaagacaccaatggccacaggagctgtcccagctaagaagaaagtggtccaggctaccaaatcccctgcctcttccccacaccccaccacacgaagaaggcaaaggctgaaggcctctgagttcaagtctgagcctcggtgggattttgaggaggaatatagcttggatatgagcagcctgcagacgaactgctctgcttctgtgaagatcaaggcctccaagtcaccatggctacagaatatctttctgcccaacatcactctgttcctggactctggacgcttcacccagagcgagtggaaccgtctagagcacttcgctcctccctttggcttcatggaactcaatcagtccctggtacagaaggtggtgacccgcttccctccagttcgccagcagcagctcctcctggccagcctccctactgggtactccaagtgtatcacctgtgctgtagtgggcaacgggggcatcctgaatgattcacgtgttggccgggagatagacagccatgactatgttttccgactgagtggagccgttattaaaggatatgaacaggatgtggggacccggacatccttctatggcttcactgctttctctctgacccagtctatcctcattttgggtagacgaggcttccagcatgtgcctctgggaaaggacgtccgatatctacacttcctggaaggcacccgggactatgagtggctggaagctatgtttttgaatcagaccttggcaaaaacccacctttcctggttcaggcataggcctcaggaagccttccggaatgccttggacttggatcgatacttgctgctgcacccagactttctccgttacatgaagaacaggtttctgaggtcaaagaccctggacactgcccattggagaatataccgccccaccactggtgccctcctgctgctcacagcccttcatctctgtgacaaggtcagcgcctatggcttcatcaccgagggccaccagcgcttctctgaccactactatgatacatcatggaaacggctcatcttttatatcaaccatgacttcaggttagagagaatggtgtggaagcggttgcatgatgaaggcatcatctggctctaccagcgtccacaaagtgacaaagcaaagaac

SEQ ID NO: 18 - protein, 495 aa, Artificial Sequence
  Description of Artificial Sequence:mouse N-acetylgalactosamine-alpha2,6-sialyltransferase I (ST6GalNAcI) beginning at residue 32 of the native mouse protein, mouse delta31
DPRAKDSRCQFIWKNDASAQENQQKAEPQVPIMTLSPRVHNKESTSVSSKDLKKQEREAVQGEQAEGKEKRKLETIRPAPENPQSKAEPAAKTPVSEHLDKLPRTPGALSTRKTPMATGAVPAKKKVVQATKSPASSPHPTTRRRQRLKASEFKSEPRWDFEEEYSLDMSSLQTNCSASVKIKASKSPWLQNIFLPNITLFLDSGRFTQSEWNRLEHFAPPFGFMELNQSLVQKVVTRFPPVRQQQLLLASLPTGYSKCITCAVVGNGGILNDSRVGREIDSHDYVFRLSGAVIKGYEQDVGTRTSFYGFTAFSLTQSILILGRRGFQHVPLGKDVRYLHFLEGTRDYEWLEAMFLNQTLAKTHLSWFRHRPQEAFRNALDLDRYLLLHPDFLRYMKNRFLRSKTLDTAHWRIYRPTTGALLLLTALHLCDKVSAYGFITEGHQRFSDHYYDTSWKRLIFYINHDFRLERMVWKRLHDEGIIWLYQRPQSDKAKN

SEQ ID NO: 19 - DNA, 168 bp, Artificial Sequence
  Description of Artificial Sequence:gp67 from plasmid pAcGP67-B baculovirus transfer vector cloning site
atgctactagtaaatcagtcacaccaaggcttcaataaggaacacacaagcaagatggtaagcgctattgttttatatgtgcttttggcggcggcggcgcattctgcctttgcggcggatcttggatcccgggccatgggaattccggagcggccgctgcagatctga

SEQ ID NO: 20 - protein, 55 aa, Artificial Sequence
  Description of Artificial Sequence:gp67 from plasmid pAcGP67-B baculovirus transfer vector cloning site
  SIGNAL (1)..(38): gp67 secretion signal sequence
MLLVNQSHQGFNKEHTSKMVSAIVLYVLLAAAAHSAFAADLGSRAMGIPERPLQI

SEQ ID NO: 21 - DNA, 1200 bp, Artificial Sequence
  Description of Artificial Sequence:mouse N-acetylgalactosamine-alpha2,6-sialyltransferase I (ST6GalNAcI) beginning at residue 127 of the native mouse protein, mouse delta126
tcagaacacctggacaaactacccagaactccaggagcactgtcaacaaggaagacaccaatggccacaggagctgtcccagctaagaagaaagtggtccaggctaccaaatcccctgcctcttccccacaccccaccacacgaagaaggcaaaggctgaaggcctctgagttcaagtctgagcctcggtgggattttgaggaggaatatagcttggatatgagcagcctgcagacgaactgctctgcttctgtgaagatcaaggcctccaagtcaccatggctacagaatatctttctgcccaacatcactctgttcctggactctggacgcttcacccagagcgagtggaaccgtctagagcacttcgctcctccctttggcttcatggaactcaatcagtccctggtacagaaggtggtgacccgcttccctccagttcgccagcagcagctcctcctggccagcctccctactgggtactccaagtgtatcacctgtgctgtagtgggcaacgggggcatcctgaatgattcacgtgttggccgggagatagacagccatgactatgttttccgactgagtggagccgttattaaaggatatgaacaggatgtggggacccggacatccttctatggcttcactgctttctctctgacccagtctatcctcattttgggtagacgaggcttccagcatgtgcctctgggaaaggacgtccgatatctacacttcctggaaggcacccgggactatgagtggctggaagctatgtttttgaatcagaccttggcaaaaacccacctttcctggttcaggcataggcctcaggaagccttccggaatgccttggacttggatcgatacttgctgctgcacccagactttctccgttacatgaagaacaggtttctgaggtcaaagaccctggacactgcccattggagaatataccgccccaccactggtgccctcctgctgctcacagcccttcatctctgtgacaaggtcagcgcctatggcttcatcaccgagggccaccagcgcttctctgaccactactatgatacatcatggaaacggctcatcttttatatcaaccatgacttcaggttagagagaatggtgtggaagcggttgcatgatgaaggcatcatctggctctaccagcgtccacaaagtgacaaagcaaagaac

SEQ ID NO: 22 - protein, 400 aa, Artificial Sequence
  Description of Artificial Sequence:mouse N-acetylgalactosamine-alpha2,6-sialyltransferase I (ST6GalNAcI) beginning at residue 127 of the native mouse protein, mouse delta126
SEHLDKLPRTPGALSTRKTPMATGAVPAKKKVVQATKSPASSPHPTTRRRQRLKASEFKSEPRWDFEEEYSLDMSSLQTNCSASVKIKASKSPWLQNIFLPNITLFLDSGRFTQSEWNRLEHFAPPFGFMELNQSLVQKVVTRFPPVRQQQLLLASLPTGYSKCITCAVVGNGGILNDSRVGREIDSHDYVFRLSGAVIKGYEQDVGTRTSFYGFTAFSLTQSILILGRRGFQHVPLGKDVRYLHFLEGTRDYEWLEAMFLNQTLAKTHLSWFRHRPQEAFRNALDLDRYLLLHPDFLRYMKNRFLRSKTLDTAHWRIYRPTTGALLLLTALHLCDKVSAYGFITEGHQRFSDHYYDTSWKRLIFYINHDFRLERMVWKRLHDEGIIWLYQRPQSDKAKN

SEQ ID NO: 23 - DNA, 1023 bp, Artificial Sequence
  Description of Artificial Sequence:mouse N-acetylgalactosamine-alpha2,6-sialyltransferase I (ST6GalNAcI) beginning at residue 186 of the native mouse protein, mouse delta185
tctgagcctcggtgggattttgaggaggaatatagcttggatatgagcagcctgcagacgaactgctctgcttctgtgaagatcaaggcctccaagtcaccatggctacagaatatctttctgcccaacatcactctgttcctggactctggacgcttcacccagagcgagtggaaccgtctagagcacttcgctcctccctttggcttcatggaactcaatcagtccctggtacagaaggtggtgacccgcttccctccagttcgccagcagcagctcctcctggccagcctccctactgggtactccaagtgtatcacctgtgctgtagtgggcaacgggggcatcctgaatgattcacgtgttggccgggagatagacagccatgactatgttttccgactgagtggagccgttattaaaggatatgaacaggatgtggggacccggacatccttctatggcttcactgctttctctctgacccagtctatcctcattttgggtagacgaggcttccagcatgtgcctctgggaaaggacgtccgatatctacacttcctggaaggcacccgggactatgagtggctggaagctatgtttttgaatcagaccttggcaaaaacccacctttcctggttcaggcataggcctcaggaagccttccggaatgccttggacttggatcgatacttgctgctgcacccagactttctccgttacatgaagaacaggtttctgaggtcaaagaccctggacactgcccattggagaatataccgccccaccactggtgccctcctgctgctcacagcccttcatctctgtgacaaggtcagcgcctatggcttcatcaccgagggccaccagcgcttctctgaccactactatgatacatcatggaaacggctcatcttttatatcaaccatgacttcaggttagagagaatggtgtggaagcggttgcatgatgaaggcatcatctggctctaccagcgtccacaaagtgacaaagcaaagaac

SEQ ID NO: 24 - protein, 341 aa, Artificial Sequence
  Description of Artificial Sequence:mouse N-acetylgalactosamine-alpha2,6-sialyltransferase I (ST6GalNAcI) beginning at residue 186 of the native mouse protein, mouse delta185
SEPRWDFEEEYSLDMSSLQTNCSASVKIKASKSPWLQNIFLPNITLFLDSGRFTQSEWNRLEHFAPPFGFMELNQSLVQKVVTRFPPVRQQQLLLASLPTGYSKCITCAVVGNGGILNDSRVGREIDSHDYVFRLSGAVIKGYEQDVGTRTSFYGFTAFSLTQSILILGRRGFQHVPLGKDVRYLHFLEGTRDYEWLEAMFLNQTLAKTHLSWFRHRPQEAFRNALDLDRYLLLHPDFLRYMKNRFLRSKTLDTAHWRIYRPTTGALLLLTALHLCDKVSAYGFITEGHQRFSDHYYDTSWKRLIFYINHDFRLERMVWKRLHDEGIIWLYQRPQSDKAKN

SEQ ID NO: 25 - DNA, 1782 bp, Artificial Sequence
  Description of Artificial Sequence:mouse ST6GalNAcI from pTS103
tcataccgtcccaccatcgggcgcggatctatgctactagtaaatcagtcacaccaaggcttcaataaggaacacacaagcaagatggtaagcgctattgttttatatgtgcttttggcggcggcggcgcattctgcctttgcggcggatccaagggcaaaagattccaggtgccagttcatatggaagaatgacgcaagtgcccaggagaatcaacaaaaggctgagccccaagtacccatcatgacactgtcacccagagttcacaacaaggagtcaacctctgtcagttcaaaggacctgaagaaacaggagagagaggcagtccaaggagagcaagctgaggggaaggagaagaggaagttagagaccataaggccagcaccagagaatcctcagagcaaggcagagcctgctgcaaagacgcctgtgtcagaacacctggacaaactacccagaactccaggagcactgtcaacaaggaagacaccaatggccacaggagctgtcccagctaagaagaaagtggtccaggctaccaaatcccctgcctcttccccacaccccaccacacgaagaaggcaaaggctgaaggcctctgagttcaagtctgagcctcggtgggattttgaggaggaatatagcttggatatgagcagcctgcagacgaactgctctgcttctgtgaagatcaaggcctccaagtcaccatggctacagaatatctttctgcccaacatcactctgttcctggactctggacgcttcacccagagcgagtggaaccgtctagagcacttcgctcctccctttggcttcatggaactcaatcagtccctggtacagaaggtggtgacccgcttccctccagttcgccagcagcagctcctcctggccagcctccctactgggtactccaagtgtatcacctgtgctgtagtgggcaacgggggcatcctgaatgattcacgtgttggccgggagatagacagccatgactatgttttccgactgagtggagccgttattaaaggatatgaacaggatgtggggacccggacatccttctatggcttcactgctttctctctgacccagtctatcctcattttgggtagacgaggcttccagcatgtgcctctgggaaaggacgtccgatatctacacttcctggaaggcacccgggactatgagtggctggaagctatgtttttgaatcagaccttggcaaaaacccacctttcctggttcaggcataggcctcaggaagccttccggaatgccttggacttggatcgatacttgctgctgcacccagactttctccgttacatgaagaacaggtttctgaggtcaaagaccctggacactgcccattggagaatataccgccccaccactggtgccctcctgctgctcacagcccttcatctctgtgacaaggtcagcgcctatggcttcatcaccgagggccaccagcgcttctctgaccactactatgatacatcatggaaacggctcatcttttatatcaaccatgacttcaggttagagagaatggtgtggaagcggttgcatgatgaaggcatcatctggctctaccagcgtccacaaagtgacaaagcaaagaacggcggccgcgaacaaaaactcatctcagaagaggatctgtgagatctgatcctttcctgggacccggcaagaaccaaaaactcactctcttcaaggaaatccgtaatgttaaacccgacacgatgaagcttgtcgttggatggaaaggaa

SEQ ID NO: 26 - skipped

SEQ ID NO: 27 - DNA, 1554 bp, Artificial Sequence
  Description of Artificial Sequence:chicken delta48 N-acetylgalactosamine-alpha2,6-sialyltransferase I (ST6GalNAcI)
caaagcattgcacatatgctacaagtggacacccaagatcagcaaggttcaaactattctgctaatgggagaatttcaaaggttggtttggagagagacattgcatggctcgaactgaatactgctgtgagtacaccaagtggggaagggaaggaagagcagaagaaaacagtgaaaccagttgccaaggtggaagaagccaaggagaaagtgactgtgaaaccattccctgaggtgatggggatcacaaatacaacagcatcaacagcctctgtggtggagagaacaaaggagaaaacaacagcgagaccagttccaggggtgggggaagctgatgggaagagaacaacgatagcacttcccagcatgaaggaagacaaagagaaggcgactgtgaaaccatcctttgggatgaaggtagctcatgcaaacagcacatccaaagataaaccaaaggcagaagagcctcctgcatcagtgaaagccataagacctgtgactcaggctgccacagtgacagagaagaagaaactgagggctgctgacttcaagactgagccacagtgggattttgatgatgagtacatactggatagctcatctccagtatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcc

SEQ ID NO: 28 - protein, 518 aa, Artificial Sequence
  Description of Artificial Sequence:chicken delta48 N-acetylgalactosamine-alpha2,6-sialyltransferase I (ST6GalNAcI)
QSIAHMLQVDTQDQQGSNYSANGRISKVGLERDIAWLELNTAVSTPSGEGKEEQKKTVKPVAKVEEAKEKVTVKPFPEVMGITNTTASTASVVERTKEKTTARPVPGVGEADGKRTTIALPSMKEDKEKATVKPSFGMKVAHANSTSKDKPKAEEPPASVKAIRPVTQAATVTEKKKLRAADFKTEPQWDFDDEYILDSSSPVSTCSESVRAKAAKSDWLRDLFLPNITLFIDKSYFNVSEWDRLEHFAPPYGFMELNYSLVEEVMSRLPPNPHQQLLLANSSSNVSTCISCAVVGNGGILNNSGMGQEIDSHDYVFRVSGAVIKGYEKDVGTKTSFYGFTAYSLVSSLQNLGHKGFKKIPQGKHIRYIHFLEAVRDYEWLKALLLDKDIRKGFLNYYGRRPRERFDEDFTMNKYLVAHPDFLRYLKNRFLKSKNLQKPYWRLYRPTTGALLLLTALHLCDRVSAYGYITEGHQKYSDHYYDKEWKRLVFYVNHDFNLEKQVWKRLHDENIMKLYQRS

SEQ ID NO: 29 - DNA, 1542 bp, Artificial Sequence
  Description of Artificial Sequence:chicken delta152 N-acetylgalactosamine-alpha2,6-sialyltransferase I (ST6GalNAcI)
catatgctacaagtggacacccaagatcagcaaggttcaaactattctgctaatgggagaatttcaaaggttggtttggagagagacattgcatggctcgaactgaatactgctgtgagtacaccaagtggggaagggaaggaagagcagaagaaaacagtgaaaccagttgccaaggtggaagaagccaaggagaaagtgactgtgaaaccattccctgaggtgatggggatcacaaatacaacagcatcaacagcctctgtggtggagagaacaaaggagaaaacaacagcgagaccagttccaggggtgggggaagctgatgggaagagaacaacgatagcacttcccagcatgaaggaagacaaagagaaggcgactgtgaaaccatcctttgggatgaaggtagctcatgcaaacagcacatccaaagataaaccaaaggcagaagagcctcctgcatcagtgaaagccataagacctgtgactcaggctgccacagtgacagagaagaagaaactgagggctgctgacttcaagactgagccacagtgggattttgatgatgagtacatactggatagctcatctccagtatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcc

SEQ ID NO: 30 - protein, 514 aa, Artificial Sequence
  Description of Artificial Sequence:chicken delta152 N-acetylgalactosamine-alpha2,6-sialyltransferase I (ST6GalNAcI)
HMLQVDTQDQQGSNYSANGRISKVGLERDIAWLELNTAVSTPSGEGKEEQKKTVKPVAKVEEAKEKVTVKPFPEVMGITNTTASTASVVERTKEKTTARPVPGVGEADGKRTTIALPSMKEDKEKATVKPSFGMKVAHANSTSKDKPKAEEPPASVKAIRPVTQAATVTEKKKLRAADFKTEPQWDFDDEYILDSSSPVSTCSESVRAKAAKSDWLRDLFLPNITLFIDKSYFNVSEWDRLEHFAPPYGFMELNYSLVEEVMSRLPPNPHQQLLLANSSSNVSTCISCAVVGNGGILNNSGMGQEIDSHDYVFRVSGAVIKGYEKDVGTKTSFYGFTAYSLVSSLQNLGHKGFKKIPQGKHIRYIHFLEAVRDYEWLKALLLDKDIRKGFLNYYGRRPRERFDEDFTMNKYLVAHPDFLRYLKNRFLKSKNLQKPYWRLYRPTTGALLLLTALHLCDRVSAYGYITEGHQKYSDHYYDKEWKRLVFYVNHDFNLEKQVWKRLHDENIMKLYQRS

SEQ ID NO: 31 - DNA, 1020 bp, Artificial Sequence
  Description of Artificial Sequence:chicken delta226 N-acetylgalactosamine-alpha2,6-sialyltransferase I (ST6GalNAcI)
agggctgctgacttcaagactgagccacagtgggattttgatgatgagtacatactggatagctcatctccagtatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcc

SEQ ID NO: 32 - protein, 340 aa, Artificial Sequence
  Description of Artificial Sequence:chicken delta226 N-acetylgalactosamine-alpha2,6-sialyltransferase I (ST6GalNAcI)
RAADFKTEPQWDFDDEYILDSSSPVSTCSESVRAKAAKSDWLRDLFLPNITLFIDKSYFNVSEWDRLEHFAPPYGFMELNYSLVEEVMSRLPPNPHQQLLLANSSSNVSTCISCAVVGNGGILNNSGMGQEIDSHDYVFRVSGAVIKGYEKDVGTKTSFYGFTAYSLVSSLQNLGHKGFKKIPQGKHIRYIHFLEAVRDYEWLKALLLDKDIRKGFLNYYGRRPRERFDEDFTMNKYLVAHPDFLRYLKNRFLKSKNLQKPYWRLYRPTTGALLLLTALHLCDRVSAYGYITEGHQKYSDHYYDKEWKRLVFYVNHDFNLEKQVWKRLHDENIMKLYQRS

SEQ ID NO: 33 - DNA, 1005 bp, Artificial Sequence
  Description of Artificial Sequence:chicken delta232 N-acetylgalactosamine-alpha2,6-sialyltransferase I (ST6GalNAcI)
aagactgagccacagtgggattttgatgatgagtacatactggatagctcatctccagtatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcc

SEQ ID NO: 34 - protein, 335 aa, Artificial Sequence
  Description of Artificial Sequence:chicken delta232 N-acetylgalactosamine-alpha2,6-sialyltransferase I (ST6GalNAcI)
KTEPQWDFDDEYILDSSSPVSTCSESVRAKAAKSDWLRDLFLPNITLFIDKSYFNVSEWDRLEHFAPPYGFMELNYSLVEEVMSRLPPNPHQQLLLANSSSNVSTCISCAVVGNGGILNNSGMGQEIDSHDYVFRVSGAVIKGYEKDVGTKTSFYGFTAYSLVSSLQNLGHKGFKKIPQGKHIRYIHFLEAVRDYEWLKALLLDKDIRKGFLNYYGRRPRERFDEDFTMNKYLVAHPDFLRYLKNRFLKSKNLQKPYWRLYRPTTGALLLLTALHLCDRVSAYGYITEGHQKYSDHYYDKEWKRLVFYVNHDFNLEKQVWKRLHDENIMKLYQRS

SEQ ID NO: 35 - DNA, 300 bp, Artificial Sequence
  Description of Artificial Sequence:portion of polycloning sites of pFastBac-1-gp vector with polyhedrin promoter and SV40 polyadenylation signal
tagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttg

SEQ ID NO: 36 - DNA, 299 bp, Artificial Sequence
  Description of Artificial Sequence:portion of polycloning sites of pFastBac-1-gp vector with gp67 sequence
gattattcataccgtcccaccatcgggcgcggatcccggtccgaaaccatgctactagtaaatcagtcacaccaaggcttcaataaggaacacacaagcaagatggtaagcgctattgttttatatgtgcttttggcggcggcggcgcattctgcctttgcggcggatcttggaattcaaaggcctacgtcgacgagctcactagtcgcggccgctttcgaatctagagcctgcagtctcgaggcatgcggtaccaagcttgtcgagaagtactagaggatcataatcagccataccac

SEQ ID NO: 37 - protein, 75 aa, Artificial Sequence
  Description of Artificial Sequence:translation of portion of polycloning sites of pFastBac-1-gp vector with gp67 sequence
MLLVNQSHQGFNKEHTSKMVSAIVLYVLLAAAAHSAFAADLGIQRPTSTSSLVAAAFESRACSLEACGTKLVEKY

SEQ ID NO: 38 - DNA, 1029 bp, Artificial Sequence
  Description of Artificial Sequence:chicken delta232 N-acetylgalactosamine-alpha2,6-sialyltransferase I (delta232 ST6GalNAcI)
gaattcggatccacggagccacagtgggattttgatgatgagtacatactggatagctcatctccagcatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcctgactcgagggaatt

SEQ ID NO: 39 - protein, 334 aa, Artificial Sequence
  Description of Artificial Sequence:chicken delta232 N-acetylgalactosamine-alpha2,6-sialyltransferase I (delta232 ST6GalNAcI)
TEPQWDFDDEYILDSSSPASTCSESVRAKAAKSDWLRDLFLPNITLFIDKSYFNVSEWDRLEHFAPPYGFMELNYSLVEEVMSRLPPNPHQQLLLANSSSNVSTCISCAVVGNGGILNNSGMGQEIDSHDYVFRVSGAVIKGYEKDVGTKTSFYGFTAYSLVSSLQNLGHKGFKKIPQGKHIRYIHFLEAVRDYEWLKALLLDKDIRKGFLNYYGRRPRERFDEDFTMNKYLVAHPDFLRYLKNRFLKSKNLQKPYWRLYRPTTGALLLLTALHLCDRVSAYGYITEGHQKYSDHYYDKEWKRLVFYVNHDFNLEKQVWKRLHDENIMKLYQRS

SEQ ID NO: 40 - DNA, 1701 bp, Gallus gallus
  full-length chicken N-acetylgalactosamine-alpha2,6-sialyltransferase I (ST6GalNAcI)
atggggtttttaatcagaaggcttcctaaagattccagaatattccgttggctccttattttaacagtcttttccttcatcattactagttttagcgccttgtttggcatggagaaaagcattttcaggcagctcaagatttaccaaagcattgcacatatgctacaagtggacacccaagatcagcaaggttcaaactattctgctaatgggagaatttcaaaggttggtttggagagagacattgcatggctcgaactgaatactgctgtgagtacaccaagtggggaagggaaggaagagcagaagaaaacagtgaaaccagttgccaaggtggaagaagccaaggagaaagtgactgtgaaaccattccctgaggtgatggggatcacaaatacaacagcatcaacagcctctgtggtggagagaacaaaggagaaaacaacagcgagaccagttccaggggtgggggaagctgatgggaagagaacaacgatagcacttcccagcatgaaggaagacaaagagaaggcgactgtgaaaccatcctttgggatgaaggtagctcatgcaaacagcacatccaaagataaaccaaaggcagaagagcctcctgcatcagtgaaagccataagacctgtgactcaggctgccacagtgacagagaagaagaaactgagggctgctgacttcaagactgagccacagtgggattttgatgatgagtacatactggatagctcatctccagtatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcctga

SEQ ID NO: 41 - DNA, 1008 bp, Artificial Sequence
  Description of Artificial Sequence:K232 truncated chicken N-acetylgalactosamine-alpha2,6-sialyltransferase I (K232 ST6GalNAcI)
aagactgagccacagtgggattttgatgatgagtacatactggatagctcatctccagtatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcctga

SEQ ID NO: 42 - protein, 335 aa, Artificial Sequence; identical to SEQ ID NO: 34
  Description of Artificial Sequence:K232 truncated chicken N-acetylgalactosamine-alpha2,6-sialyltransferase I (K232 ST6GalNAcI)

SEQ ID NO: 43 - DNA, 32 bp, Artificial Sequence
  Description of Artificial Sequence:D32-HindIII PCR amplification primer to clone N-terminal truncated mouse ST6GalNAcI gene
taatataagcttgatccaagggcaaaagattc

SEQ ID NO: 44 - DNA, 32 bp, Artificial Sequence
  Description of Artificial Sequence:E52-BamHI PCR amplification primer to clone N-terminal truncated mouse ST6GalNAcI gene
taataaggatccgagattctgcaaaaggctga

SEQ ID NO: 45 - DNA, 32 bp, Artificial Sequence
  Description of Artificial Sequence:S127-BamHI PCR amplification primer to clone N-terminal truncated mouse ST6GalNAcI gene
taatatggatcctcagaacacctggacaaagt

SEQ ID NO: 46 - DNA, 32 bp, Artificial Sequence
  Description of Artificial Sequence:S186-BamHI PCR amplification primer to clone N-terminal truncated mouse ST6GalNAcI gene
taatatggatcctctgagcctcggtgggattt

SEQ ID NO: 47 - DNA, 32 bp, Artificial Sequence
  Description of Artificial Sequence:S201-BamHI PCR amplification primer to clone N-terminal truncated mouse ST6GalNAcI gene
taataaggatccagcagcctgcagacgaactg

SEQ ID NO: 48 - DNA, 36 bp, Artificial Sequence
  Description of Artificial Sequence:M-XhoI PCR amplification primer to clone mouse ST6GalNAcI gene
tagcgcctcgagtcagttctttgctttgtcactttg

SEQ ID NO: 49 - DNA, 24 bp, Artificial Sequence
  Description of Artificial Sequence:hST6GN1-F1 PCR primer for cloning full length human ST6GalNAcI cDNA
caggatccacatgcagaaccttcc

SEQ ID NO: 50 - DNA, 55 bp, Artificial Sequence
  Description of Artificial Sequence:hST6GN1-R2 PCR primer for cloning full length human ST6GalNAcI cDNA
gtcccgggtgccttccaggaagtgcaagtagcggacgtccttcccaagaggcacg

SEQ ID NO: 51 - DNA, 16 bp, Artificial Sequence
  Description of Artificial Sequence:hST6GN1-F2 PCR primer for cloning full length human ST6GalNAcI cDNA
ggaaggcacccgggac

SEQ ID NO: 52 - DNA, 24 bp, Artificial Sequence
  Description of Artificial Sequence:hST6GN1-R1 PCR primer for cloning full length human ST6GalNAcI cDNA
ccgaattccggtcagttcttggct

SEQ ID NO: 53 - DNA, 23 bp, Artificial Sequence
  Description of Artificial Sequence:hST6-K36-5′ primer for generating K36 clone truncation of hST6GalNAcI for expression in Sf9 cells
ccaggatccaaggagcctcaaac

SEQ ID NO: 54 - DNA, 27 bp, Artificial Sequence
  Description of Artificial Sequence:hST6-K125-5′ primer for generating truncation of hST6GalNAcI for expression in Sf9 cells
ccaggatccaagagcccagaaaaagag

SEQ ID NO: 55 - DNA, 24 bp, Artificial Sequence
  Description of Artificial Sequence:hST6-S258-5′ primer for generating S258 clone truncation of hST6GalNAcI for expression in Sf9 cells
ccaggatcctctgagcctcggtgg

SEQ ID NO: 56 - DNA, 25 bp, Artificial Sequence
  Description of Artificial Sequence:S127-EcoRI-5′ primer for cloning S127 truncated mouse mST6GalNAcI
cggaattctctcagaacacctggac

SEQ ID NO: 57 - DNA, 25 bp, Artificial Sequence
  Description of Artificial Sequence:S186-EcoRI-5′ primer for cloning S186 truncated mouse mST6GalNAcI
cggaattctctctgagcctcggtgg

SEQ ID NO: 58 - DNA, 26 bp, Artificial Sequence
  Description of Artificial Sequence:mST6-XhoI-3′ primer for cloning truncated mouse mST6GalNAcI
gcctcgagtcagttctttgctttgtc

SEQ ID NO: 59 - DNA, 34 bp, Artificial Sequence
  Description of Artificial Sequence:PCR primer ch233BamHI2 for isolation of chicken ST6GalNAcI, PCR primer to generate delta232 mutant
gattcgggatccacggagccacagtgggattttg

SEQ ID NO: 60-61 - skipped (2 entries)

SEQ ID NO: 62 - DNA, 31 bp, Artificial Sequence
  Description of Artificial Sequence:delta48BamHI PCR primer to generate delta48 mutant
ggatcccaaagtattgcacacatgctacaag

SEQ ID NO: 63 - DNA, 31 bp, Artificial Sequence
  Description of Artificial Sequence:S566EcoRI PCR primer to generate delta48 and delta152 mutants
ggcgaattctcacgatctctggtagagtttc

SEQ ID NO: 64 - DNA, 26 bp, Artificial Sequence
  Description of Artificial Sequence:delta152BamHI PCR primer to generate delta152 mutant
ggatccgttccaggtgtgggagaagc

SEQ ID NO: 65 - DNA, 29 bp, Artificial Sequence
  Description of Artificial Sequence:delta225BamHI PCR primer to generate delta225 mutant
ggatccctgagggctgctgacttcaagac

SEQ ID NO: 66 - DNA, 38 bp, Artificial Sequence
  Description of Artificial Sequence:PCR primer to generate delta225 mutant
ggtgcttaagagtaatgctagagaccatctcaaagtac

SEQ ID NO: 67 - protein, 600 aa, Homo sapiens
  human N-acetylgalactosamine-alpha2,6-sialyltransferase I (ST6GalNAcI)
MRSCLWRCRHLSQGVQWSLLLAVLVFFLFALPSFIKEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 68 - protein, 526 aa, Artificial Sequence
  mouse N-acetylgalactosamine-alpha2,6-sialyltransferase I (ST6GalNAcI)
  MOD_RES (441): Xaa = any amino acid
MTRYCRGLSQRQAFLLLTVLALLFILLFVVKDPRAKDSRRQFILNNDSSAQEILQKAEPQGPIMTLSPRVHNKEATSVSSKDLKKQEREAVQGEQAEGKEKRKLETIRPAPENPQSKAEPAAKTPVSEHLDKVPRTPGALSTRKTPMATGAVPAKKKVVQATKSPASSPHPTTRRRQRLKASEFKSEPRWDFEEEYSLDMSSLQTNCSASVKIKASKSPWLQNIFLPNITLFLDSGRFTQSEWNRLEHFAPPFGFMELNQSLVQKVVTRFPPVRQQQLLLASLPTGYSKCITCAVVGNGGILNDSRVGREIDSHDYVFRLSGAVIKGYEQDVGTRTSFYGFTAFSLTQSILILGRRGFQHVPLGKDVRYLHFLEGTRNYEWLEAMFLNQTLAKTHLSWFRHRPQEAFRNALDLDRYLLLHPDFLRYMKNRFLRSKTLDTAXWRIYRPTTGALLLLTALHLCDKVSAYGFITEGHERFSDHYYDTSWKRLIFYINHDFRLERMVWKRLHDEGIIWLYQRPQSDKAKN

SEQ ID NO: 69 - protein, 4 aa, Artificial Sequence
  Description of Artificial Sequence:consensus peptide and primary consensus peptide
MTNT

SEQ ID NO: 70 - protein, 7 aa, Artificial Sequence
  Description of Artificial Sequence:consensus peptide
RKLTTIR

SEQ ID NO: 71 - protein, 4 aa, Artificial Sequence
  Description of Artificial Sequence:consensus peptide
KAAT

SEQ ID NO: 72 - protein, 4 aa, Artificial Sequence
  Description of Artificial Sequence:consensus peptide
STRK

SEQ ID NO: 73 - protein, 6 aa, Artificial Sequence
  Description of Artificial Sequence:consensus peptide
QRLKAA

SEQ ID NO: 74 - protein, 9 aa, Artificial Sequence
  Description of Artificial Sequence:consensus peptide
FKSEPRWDF

SEQ ID NO: 75 - protein, 16 aa, Artificial Sequence
  Description of Artificial Sequence:primary consensus peptide
FKSEPRWDFEEEYSLD

SEQ ID NO: 76 - protein, 8 aa, Artificial Sequence
  Description of Artificial Sequence:consensus peptide and primary consensus peptide
SSLQTTCS

SEQ ID NO: 77 - protein, 9 aa, Artificial Sequence
  Description of Artificial Sequence:consensus peptide and primary consensus peptide
SVKIKASKS

SEQ ID NO: 78 - protein, 12 aa, Artificial Sequence
  Description of Artificial Sequence:consensus peptide and primary consensus peptide
LFLPNITLFLDS

SEQ ID NO: 79 - protein, 6 aa, Artificial Sequence
  Description of Artificial Sequence:consensus peptide
FNQSEW

SEQ ID NO: 80 - protein, 47 aa, Artificial Sequence
  Description of Artificial Sequence:primary consensus peptide
FNQSEWDRLEHFAPPFGFMELNYSLVQKVVTRFPPVPQQQLLLASLP

SEQ ID NO: 81 - protein, 8 aa, Artificial Sequence
  Description of Artificial Sequence:consensus peptide
RLEHFAPP

SEQ ID NO: 82 - protein, 10 aa, Artificial Sequence
  Description of Artificial Sequence:consensus peptide
GFMELNYSLV

SEQ ID NO: 83 - protein, 20 aa, Artificial Sequence
  Description of Artificial Sequence:consensus peptide
KVVTRFPPVPQQQLLLASLP

SEQ ID NO: 84 - protein, 14 aa, Artificial Sequence
  Description of Artificial Sequence:consensus peptide
CITCAVVGNGGILN

SEQ ID NO: 85 - protein, 16 aa, Artificial Sequence
  Description of Artificial Sequence:primary consensus peptide
CITCAVVGNGGILNNS

SEQ ID NO: 86 - protein, 37 aa, Artificial Sequence
  Description of Artificial Sequence:consensus peptide
MGQEIDSHDYVFRLSGAVIKGYEQDVGTRTSFYGFTA

SEQ ID NO: 87 - protein, 48 aa, Artificial Sequence
  Description of Artificial Sequence:primary consensus peptide
MGQEIDSHDYVFRLSGAVIKGYEQDVGTRTSFYGFTAFSLTQSLLILG

SEQ ID NO: 88 - protein, 10 aa, Artificial Sequence
  Description of Artificial Sequence:consensus peptide
SLTQSLLILG

SEQ ID NO: 89 - protein, 4 aa, Artificial Sequence
  Description of Artificial Sequence:consensus peptide
RGFK

SEQ ID NO: 90 - protein, 30 aa, Artificial Sequence
  Description of Artificial Sequence:primary consensus peptide
VPLGKDVRYLHFLEGTRDYEWLEALLLNQT

SEQ ID NO: 91 - protein, 5 aa, Artificial Sequence
  Description of Artificial Sequence:consensus peptide
PLGKD

SEQ ID NO: 92 - protein, 10 aa, Artificial Sequence
  Description of Artificial Sequence:consensus peptide
RYLHFLEGTR

SEQ ID NO: 93 - protein, 6 aa, Artificial Sequence
  Description of Artificial Sequence:consensus peptide
YEWLEA

SEQ ID NO: 94 - protein, 14 aa, Artificial Sequence
  Description of Artificial Sequence:primary consensus peptide
WFRHRPQEAFREAL

SEQ ID NO: 95 - protein, 9 aa, Artificial Sequence
  Description of Artificial Sequence:consensus peptide
RHRPQEAFR

SEQ ID NO: 96 - protein, 26 aa, Artificial Sequence
  Description of Artificial Sequence:primary consensus peptide
MDRYLLLHPDFLRYMKNRFLRSKTLD

SEQ ID NO: 97 - protein, 12 aa, Artificial Sequence
  Description of Artificial Sequence:consensus peptide
RYLLLHPDFLRY

SEQ ID NO: 98 - protein, 10 aa, Artificial Sequence
  Description of Artificial Sequence:consensus peptide
KNRFLRSKTL

SEQ ID NO: 99 - protein, 21 aa, Artificial Sequence
  Description of Artificial Sequence:consensus peptide
WRIYRPTTGALLLLTALHLCD

SEQ ID NO: 100 - protein, 5 aa, Artificial Sequence
  Description of Artificial Sequence:consensus peptide
VSAYG

SEQ ID NO: 101 - protein, 34 aa, Artificial Sequence
  Description of Artificial Sequence:primary consensus peptide
VSAYGFITEGHERFSDHYYDTSWKRLIFYINHDF

SEQ ID NO: 102 - protein, 5 aa, Artificial Sequence
  Description of Artificial Sequence:consensus peptide
ITEGH

SEQ ID NO: 103 - protein, 12 aa, Artificial Sequence
  Description of Artificial Sequence:consensus peptide
SDHYYDTSWKRL

SEQ ID NO: 104 - protein, 4 aa, Artificial Sequence
  Description of Artificial Sequence:consensus peptide
NHDF

SEQ ID NO: 105 - protein, 11 aa, Artificial Sequence
  Description of Artificial Sequence:consensus peptide
VWKRLHDEGII

SEQ ID NO: 106 - protein, 5 aa, Artificial Sequence
  Description of Artificial Sequence:consensus peptide
LYQRP

SEQ ID NO: 107 - protein, 600 aa, Homo sapiens; identical to SEQ ID NO: 67
  human N-acetylgalactosamine-alpha2,6-sialyltransferase I (ST6GalNAcI)

SEQ ID NO: 108 - protein, 495 aa, Artificial Sequence
  Description of Artificial Sequence:mouse N-acetylgalactosamine-alpha2,6-sialyltransferase I (ST6GalNAcI) protein beginning at residue 32 of the native mouse protein
DPRAKDSRCQFIWKNDASAQENQQKAEPQVPIMTLSPRVHNKESTSVSSKDLKKQEREAVQGEQAEGKEKRKLETIRPAPENPQSKAEPAAKTPVSEHLDKLPRTPGALSTRKTPMATGAVPAKKKVVQATKSPASSPHPTTRRRQRLKASEFKSEPRWDFEEEYSLDMSSLQTNCSASVKIKASKSPWLQNIFLPNITLFLDSGRFTQSEWNRLEHFAPPFGFMELNQSLVQKVVTRFPPVRQQQLLLASLPTGYSKCITCAVVGNGGILNDSRVGREIDSHDYVFRLSGAVIKGYEQDVGTRTSFYGFTAFSLTQSILILGRRGFQHVPLGKDVRYLHFLEGTRNYEWLEAMFLNQTLAKTHLSWFRHRPQEAFRNALDLDRYLLLHPDFLRYMKNRFLRSKTLDTAHWRIYRPTTGALLLLTALHLCDKVSAYGFITEGHQRFSDHYYDTSWKRLIFYINHDFRLERMVWKRLHDEGIIWLYQRPQSDKAKN

SEQ ID NO: 109 - protein, 4 aa, Artificial Sequence
  Description of Artificial Sequence:example full-length peptide
AEKL

SEQ ID NO: 110 - protein, 5 aa, Artificial Sequence
  Description of Artificial Sequence:example N-terminally truncated form of full-length peptide
GAEKL

SEQ ID NO: 111 - DNA, 32 bp, Artificial Sequence
  Description of Artificial Sequence:hST6-T73 truncation oligo for hST6GalNAcI
tattggatccacaaccatctatgcagagccag

SEQ ID NO: 112 - DNA, 31 bp, Artificial Sequence
  Description of Artificial Sequence:hST6-E110 truncation oligo for hST6GalNAcI
tattggatccgaggagcaggacaaggtgccc

SEQ ID NO: 113 - DNA, 32 bp, Artificial Sequence
  Description of Artificial Sequence:hST6-M134 truncation oligo for hST6GalNAcI
tattggatccatggtgaacacactgtcaccca

SEQ ID NO: 114 - DNA, 32 bp, Artificial Sequence
  Description of Artificial Sequence:hST6-T171 truncation oligo for hST6GalNAcI
tattggatccaccaggaagctgacggcctcca

SEQ ID NO: 115 - DNA, 32 bp, Artificial Sequence
  Description of Artificial Sequence:hST6-A233 truncation oligo for hST6GalNAcI
tattggatccgccaccccaccccctgcccctt

SEQ ID NO: 116 - DNA, 32 bp, Artificial Sequence
  Description of Artificial Sequence:hST6-G273 truncation oligo for hST6GalNAcI
tattggatccggaggccttcagacgacttgcc

SEQ ID NO: 117 - DNA, 34 bp, Artificial Sequence
  Description of Artificial Sequence:hST6-CooH truncation oligo for hST6GalNAcI
gcgctctagatcagttcttggctttggcagttcc

SEQ ID NO: 118 - protein, 4 aa, Artificial Sequence
  Description of Artificial Sequence:four amino acid extension introduced at NotI site between starch binding domain (SBD) and ST6GalNAcI coding sequence in three way fusion in pAcGP67B vector
WRPP

SEQ ID NO: 119 - protein, 4 aa, Artificial Sequence
  Description of Artificial Sequence:four amino acid extension introduced at NotI site between starch binding domain (SBD) and ST6GalNAcI coding sequence in three way fusion in pAcGP67B vector
RRPP